(19) United States
(12) Patent Application Publication
(10) Pub. No.: US 2023/0407358 A1
(43) Pub. Date: Dec. 21, 2023

(54) IMPROVED PROCESSES FOR IN VITRO TRANSCRIPTION OF MESSENGER RNA

(71) Applicant: Translate Bio, Inc., Waltham, MA (US)

(72) Inventors: Dustin Cooper, Cambridge, MA (US); Frank DeRosa, Cambridge, MA (US); Anusha Dias, Cambridge, MA (US); Jeffrey S. Dubins, Cambridge, MA (US); Xiaobo Gu, Cambridge, MA (US); Khang Anh Tran, Cambridge, MA (US)

(21) Appl. No.: 17/800,157

(22) PCT Filed: Feb. 18, 2021
(86) PCT No.: PCT/US2021/018481

§ 371 Date: Aug. 16, 2022

Related U.S. Application Data
(60) Provisional application No. 62/978,180, filed on Feb. 18, 2020.

(51) Int. Cl. C12P 19/34; C12N 9/12; C12N 15/10

(52) U.S. Cl. CPC C12P 19/34; C12Y 207/07006; C12N 9/1247; C12N 15/1068

(57) ABSTRACT

The present invention provides methods for preparing optimized DNA sequences as templates for in vitro transcription of mRNA. These DNA sequences are optimized to avoid premature termination of transcription by RNA polymerase. The invention also provides methods for preparing optimized DNA sequences that include one or more termination signal at their 3′ end to reduce or prevent non-templated “runoff” transcription.

### CROSS REFERENCE TO RELATED APPLICATIONS

This application is a US 371 National Stage entry of PCT/US2021/018481, filed Feb. 18, 2021, and claims priority to U.S. Provisional Application Ser. No. 62/978,180, filed Feb. 18, 2020, the disclosure of which is hereby incorporated by reference in its entirety.

### INCORPORATION-BY-REFERENCE OF SEQUENCE LISTING

The present specification makes reference to a Sequence Listing (submitted electronically as a .txt file named MRT-2121WO_SL). The .txt file was generated on Dec. 11, 2022 and is 1,048,576 bytes in size. The entire contents of the sequence are herein incorporated by reference.

### BACKGROUND OF THE INVENTION

mRNA therapy becomes increasingly important for treating various diseases. It was reported that both T7 and SP6 RNA polymerases generate abortive transcripts during in vitro synthesis of mRNA (Nam et al. 1988, The Journal of Biological Chemistry, 263: 34, pp 18123-18127; Lee et al., Nucleic Acids Research 2010, 1-9). The presence of such abortive transcripts in a therapeutic composition based on in vitro synthesized mRNA could impact its safety and efficacy.

mRNA transcripts produced by T7 RNA polymerases in particular are known to be contaminated with RNAs longer and shorter than the desired transcript, for example due to “runoff” transcription generating elongated transcripts longer than the templated sequence. These non-templated elongated portions of the transcripts may anneal to the RNA molecule itself or another RNA molecule to form intra- or intermolecular RNA duplexes (Gholamalipour et al. 2018, Nucleic Acids Research, 46: 18 pp 9253-9263). RNA duplexes can be highly immunogenic (Mu et al. 2018, Nucleic Acids Research, 46: 5239-5249). The RNA duplex impurities are not efficiently removed from in vitro transcribed (IVT) mRNA using standard laboratory protocols. The most effective purification method is considered to be ion pair reversed-phase high-performance liquid chromatography (HPLC). However, this method is not scalable, requires the use of toxic reagents and is prohibitively expensive for many laboratories (Baiersdorfer et al. 2019, Molecular Therapy: Nucleic Acids, 15: 26-35). Selective binding of double-stranded RNA to cellulose in an ethanol-containing buffer has recently been identified as a scalable method for the removal of RNA duplex impurities from IVT mRNA, though this method resulted in significant reductions in RNA yield (Baiersdorfer et al. 2019, ibid.).

SP6 RNA polymerases have been used as an alternative to T7 RNA polymerase. However, incomplete mRNA transcripts remain a problem when SP6 RNA polymerases are used in in vitro transcription. It has previously been reported that SP6 RNA polymerase stops transcription at two signals (upstream and downstream signals) in the rrnB t1 terminator, and alterations in the signal regions affect termination efficiency (Kwon & Kang 1999, The Journal of Biological Chemistry, 274: 41 pp 29149-29155). The inventors discovered that rrnB t1-like termination signals are frequently present in template DNA sequences used for in vitro transcription of mRNA. In addition, they found that “runoff” transcription can also occur with SP6 RNA polymerase.

WO 2017/009376 provides a method of producing RNA from circular DNA in which the circular DNA template sequence includes an RNA polymerase promoter sequence, followed by a sequence encoding a self-cleaving ribozyme, followed by an RNA polymerase terminator sequence element. The data included with this application demonstrate that termination efficiencies of up to about 95% can be reached for in vitro transcription from a linearized DNA plasmid including a self-cleaving ribozyme and two or four terminator sequences. Termination efficiencies of this magnitude are not sufficient for commercial-scale processes employed in the production of therapeutic mRNAs.

WO 2012/170443 provides a method of producing RNA from a circular DNA template in which a phage promoter is operably linked to a sequence encoding an RNA polynucleotide of interest operably linked to a multiple terminator domain. The multiple terminator domain comprises at least three termination signals selected from class I and class II termination signals. Class I termination signals (exemplified by the Phi bacteriophage T7 terminator, also known as the T7 phi terminator) encode RNA sequences that can form a stable stem-loop structure followed by a run of six U residues. Class II termination signals (exemplified by the human preproparathyroid hormone (PTH) gene) encode an interrupted run of six U residues, but lack an apparent stem-loop structure. The rrnB t1 termination signal is a class II termination signal. Like in WO 2017/009376, the DNA templates tested in the examples of WO 2012/170443 include a sequence encoding a self-cleaving ribozyme between the sequences encoding the RNA polynucleotide of interest and a multiple terminator domain consisting of two T7 phi terminators (class I), two PTG terminators (class II) and a pBR322 terminator (class I).

Du et al. (2009, Biotechnol. Biogen., 104(6): 1189-1196) considered the large size (100 bp) and inefficiency of the T7 phi terminator to be problematic, and attempted to improve termination efficiency during transcription from a circular DNA template by instead including 1-3 vesicular stomatitis virus (vsv) class II termination signals (TATCTGTTAGTTTTTTTC (SEQ ID NO: 36)) in tandem, each separated by 8 base pairs. They found that termination efficiency was only 53-62% when a single vsv termination signal was used. Termination efficiency increased to 65-75% when 2-3 vsv terminators were used.

Accordingly, a need exists for improved in vitro transcription methods that produce full-length mRNA transcripts free of prematurely terminated transcripts and double-stranded mRNA.

### SUMMARY OF THE INVENTION

The present invention addresses this need by providing methods for preparing optimized DNA sequences as templates for in vitro transcription of mRNA. These DNA sequences are optimized to avoid premature termination of transcription by RNA polymerase. In addition, the invention also provides methods for preparing optimized DNA sequences that include one or more termination signal at their 3′ end. The termination signal reduces or prevents “runoff” transcription and thus the use of these optimized DNA sequences minimizes the formation of double-stranded mRNA transcripts.

In one aspect, the present invention relates to a method for preparing an optimized DNA sequence encoding a protein as a template for in vitro transcription, said method comprising: (a) providing a DNA sequence that comprises a protein coding sequence; (b) determining the presence of a termination signal in the DNA sequence, wherein the termination signal has the following nucleic acid sequence: 5′-X₁ATCTX₂TX₃-3′ (SEQ ID NO: 1), wherein X₁, X₂ and X₃ are independently selected from A, C, T or G; and (c) if one or more termination signal is present, modifying the DNA sequence by replacing one or more nucleic acids at any one of position 2, 3, 4, 5 and 7 of said termination signal(s) with any one of the other three nucleic acids to generate the optimized DNA sequence, wherein, if required, the one or more replacement nucleic acids are selected to preserve the amino acid sequence of the protein encoded by the protein coding sequence.

In some embodiments, steps b and c are carried out by a computer.

In some embodiments, the DNA sequence further comprises a first nucleic acid sequence encoding a 5′ UTR and/or a second nucleic acid sequence encoding a 3′ UTR.

In some embodiments, the 5 nucleotides immediately 3′ of the termination signal in the DNA sequence do not comprise 3 or more T nucleotides.

In some embodiments, the method further comprises a step of modifying the DNA sequence relative to a wildtype DNA sequence encoding the same protein sequence to optimize: (a) elements relevant to mRNA processing and stability; and/or (b) elements relevant to translation or protein folding; wherein the modifications are made before the optimized DNA sequence is generated. The elements relevant to mRNA processing or stability may include cryptic splice sites, mRNA secondary structure, stable free energy of mRNA, repetitive sequences, and RNA instability motifs. The elements relevant to translation or protein folding may include codon usage bias, codon adaptability, internal chi sites, ribosomal binding sites, premature polyA sites, Shine-Dalgarno sequences, codon context, codon-anticodon interactions, and translational pause sites.

In some embodiments, the method further comprises a step of synthesizing the optimized DNA sequence. The method may further comprise inserting the synthesized optimized DNA sequence in a nucleic acid vector for use in vitro transcription. The nucleic acid vector may comprise an RNA polymerase promoter operably linked to the optimized DNA sequence, optionally wherein the RNA polymerase is SP6 RNA polymerase or a T7 RNA polymerase. In some embodiments, the nucleic acid vector is a plasmid. The plasmid may be linearized before in vitro transcription.

In some embodiments, the method further comprises using the synthesized optimized DNA sequence in in vitro transcription to synthesize mRNA. The mRNA may be synthesized by an SP6 RNA polymerase. The SP6 RNA polymerase may be a naturally occurring SP6 RNA polymerase or a recombinant SP6 polymerase. A recombinant SP6 polymerase may comprise a tag (e.g. a his-tag). In some embodiments, the mRNA is synthesized by a T7 RNA polymerase.

In some embodiments, the method further comprises a separate step of capping and/or tailing the synthesized mRNA. In some embodiments, capping and tailing occurs during in vitro transcription.

In some embodiments, the mRNA is synthesized in a reaction mixture comprising NTPs at a concentration ranging from 1-10 mM each NTP, the DNA template at a concentration ranging from 0.01-0.5 mg/ml, and the SP6 RNA polymerase at a concentration ranging from 0.01-0.1 mg/ml. For example, the reaction mixture may comprise NTPs at a concentration of 5 mM each NTP, the DNA template at a concentration of 0.1 mg/ml, and the SP6 RNA polymerase at a concentration of 0.05 mg/ml. The NTPs may be naturally-occurring NTPs, or may comprise modified NTPs.

In some embodiments, the mRNA may be synthesized at a temperature ranging from 37-56° C.

In some embodiments, a computer program is provided comprising instructions which, when the program is executed by a computer, cause the computer to (a) receive a DNA sequence that comprises a protein coding sequence, and (b) carry out steps b and c of the methods above for preparing an optimized DNA sequence encoding a protein as a template for in vitro transcription of the invention. The invention also provides a computer-readable data carrier having stored thereon the computer program of the invention. The invention additionally provides a data carrier signal carrying the computer program of the invention. The invention additionally provides a data processing system comprising means for carry out the methods for preparing an optimized DNA sequence encoding a protein as a template for in vitro transcription of the invention.

In another aspect, the invention relates to a method for preparing an optimized DNA sequence encoding a protein as a template for in vitro transcription, said method comprising: (a) providing a DNA sequence encoding a protein; and (b) adding one or more termination signals at the 3′ end of the DNA sequence to provide the optimized DNA sequence, wherein the one or more termination signal(s) comprises the following nucleic acid sequence: 5′-X1ATCTX2TX3-3′ (SEQ ID NO: 1), wherein X₁, X₂ and X₃ are independently selected from A, C, T or G.

In some embodiments, the termination signal comprises the nucleic acid sequence 5′-X₁ATCTGTT-3′ (SEQ ID NO: 2).

In some embodiments, X₁ is T. In some embodiments X₁ is C.

In some embodiments, the termination signal is selected from 5′ TTTTATCTGTTTTTTT-3′(SEQ ID NO: 3), 5′ TTTTATCTGTTTTTTTTT-3′(SEQ ID NO: 4), 5′ CGTTTTATCTGTTTTTTT-3′ (SEQ ID NO: 5), 5′ CGTTCCATCTGTTTTTTT-3′ (SEQ ID NO: 6), 5′ CGTTTTATCTGTTTGTTT-3′ (SEQ ID NO: 7), 5′ CGTTTTATCTGTTTGTTT-3′ (SEQ ID NO: 8), or 5′ CGTTTTATCTGTTGTTTT-3′ (SEQ ID NO: 9).

In some embodiments, two or more, three or more, four or more termination signals are added to the 3′ end of the DNA sequence.

In some embodiments, the DNA sequence encoding the protein may further comprises a first nucleic acid sequence encoding a 5′ UTR and/or a second nucleic acid sequence encoding a 3′ UTR. The DNA sequence may or may not further comprise a third nucleic acid sequence encoding a poly-A tail.

In some embodiments, the DNA sequence encoding the protein does not further comprise a DNA sequence encoding a ribozyme.

In some embodiments, the 5 nucleotides immediately 3′ of the termination signal in the DNA sequence encoding the protein do not comprise 3 or more T nucleotides.

In some embodiments, the DNA sequence includes more than one termination signal, and said termination signals are separated by 10 base pairs or fewer, e.g. separated by 5-10 base pairs.

In some embodiments, the optimized DNA sequence comprises the following sequence: (a) 5′-X1ATCTX₂TX₃-(Z_{N})-X₄ATCTX₅TX₆-3′ (SEQ ID NO: 10) or (b) 5′ X₁ATCTX₂TX₃-(Z_{N})—X₄ATCTX₅TX₆-(Z_{M})-X₇ATCTX₈TX₉-3′(SEQ ID NO: 11), wherein X₁, X₂, X₃, X₄, X₅, X6, X₇, X₈ and X₉ are independently selected from A, C, T or G, Z_{N} represents a spacer sequence of N nucleotides, and Z_{M} represents a spacer sequence of M nucleotides, each of which are independently selected from A, C, T or G, and wherein N and/or M are independently 10 or fewer. In some embodiments, N is 5, 6, 7, 8, 9 or 10 and/or M is 5, 6, 7, 8, 9, 10. In some embodiments, Z is T.

In some embodiments, the method further comprises a step of modifying the DNA sequence relative to a wildtype DNA sequence encoding the same protein sequence to optimize: (a) elements relevant to mRNA processing and stability; and/or (b) elements relevant to translation or protein folding; wherein the modifications are made before the optimized DNA sequence is generated. The elements relevant to mRNA processing or stability may include cryptic splice sites, mRNA secondary structure, stable free energy of mRNA, repetitive sequences, and RNA instability motifs. The elements relevant to translation or protein folding may include codon usage bias, codon adaptability, internal chi sites, ribosomal binding sites, premature polyA sites, Shine-Dalgarno sequences, codon context, codon-anticodon interactions, and translational pause sites.

In some embodiments, the method may further comprise inserting the optimized DNA sequence into a nucleic acid vector for use in in vitro transcription.

In another aspect, the invention relates to a DNA sequence for use in in vitro transcription, comprising in 5′ to 3′ order: (a) a 5′UTR; (b) a protein coding sequence; (c) a 3′ UTR; (d) optionally a nucleic acid sequence encoding a polyA tail; and (e) a termination signal; wherein the termination signal comprises the following nucleic acid sequence: 5′-X₁ATCTX₂TX₃-3′ (SEQ ID NO: 1), wherein X1, X₂ and X₃ are independently selected from A, C, T or G. In some embodiments.

In some embodiments, X₁ is T. In some embodiments X₁ is C.

In some embodiments, the termination signal of the DNA sequence is selected from 5′ TTTTATCTGTTTTTTT-3′(SEQ ID NO: 3), 5′ TTTTATCTGTTTTTTTTT-3′(SEQ ID NO: 4), 5′ CGTTTTATCTGTTTTTTT-3′ (SEQ ID NO: 5), 5′ CGTTCCATCTGTTTTTTT-3′ (SEQ ID NO: 6), 5′ CGTTTTATCTGTTTGTTT-3′ (SEQ ID NO: 7), 5′ CGTTTTATCTGTTTGTTT-3′ (SEQ ID NO: 8), or 5′ CGTTTTATCTGTTGTTTT-3′ (SEQ ID NO: 9).

In some embodiments, the DNA sequence may comprise more than one termination signal, e.g. two or more, three or more, four or more. In some embodiments, the termination signals are separated by 10 base pairs or fewer, e.g. separated by 5-10 base pairs.

In some embodiments, the DNA sequence comprises the following sequence: (a) 5′-X1ATCTX₂TX₃-(Z_{N})-X₄ATCTX₅TX₆-3′ (SEQ ID NO: 10) or (b) 5′-X1ATCTX₂TX₃-Z_{N})-X₄ATCTX₅TX₆-(Z_{M})-X₇ATCTX₈TX₉-3′ (SEQ ID NO: 11), wherein X₁, X₂, X₃, X₄, X₅, X₆, X₇, X₈ and X₉ are independently selected from A, C, T or G, Z_{N} represents a spacer sequence of N nucleotides, and Z_{M} represents a spacer sequence of M nucleotides, each of which are independently selected from A, C, T or G, and wherein N and/or M are independently 10 or fewer. In some embodiments, N is 5, 6, 7, 8, 9 or 10 and/or M is 5, 6, 7, 8, 9 or 10. In some embodiments, Z is T.

In some embodiments, the termination signal is absent from the 5′ UTR, the protein coding sequence and the 3′ UTR of the DNA sequence.

In some embodiments, the DNA sequence encoding the protein does not further comprise a DNA sequence encoding a ribozyme.

In some embodiments, the DNA sequence is modified relative to a wildtype DNA sequence encoding the same protein sequence to optimize: (a) elements relevant to mRNA processing and stability; and/or (b) elements relevant to translation or protein folding.

In some embodiments, the invention further provides a nucleic acid vector comprising the DNA sequence of the invention. The nucleic acid vector may comprise an RNA polymerase promoter operably linked to the optimized DNA sequence, optionally wherein the RNA polymerase is SP6 RNA polymerase or a T7 RNA polymerase. In some embodiments, the nucleic acid vector is a plasmid.

In some embodiments, the invention also provides a kit for use in in vitro transcription comprising the DNA sequence or nucleic acid vector of the invention. The kit may further comprise NTPs and an RNA.

In another aspect, the invention relates to a method for the production of mRNA, said method comprising adding the nucleic acid vector of the invention to a reaction mixture comprising NTPs and an RNA polymerase, wherein the RNA polymerase transcribes the DNA sequence into mRNA transcripts. The nucleic acid vector may be a plasmid, which may or may not be linearized before in vitro transcription. The RNA polymerase may be an SP6 RNA polymerase. The SP6 RNA polymerase may be a naturally occurring SP6 RNA polymerase or a recombinant SP6 RNA polymerase. A recombinant SP6 RNA polymerase may comprise a tag (e.g., a his-tag). Alternatively, the RNA polymerase may be a T7 RNA polymerase.

In some embodiments, the method for the production of mRNA further comprises a separate step of capping and/or tailing the synthesized mRNA. In some embodiments, capping and tailing occurs during in vitro transcription.

In some embodiments, the mRNA is synthesized in a reaction mixture comprising NTPs at a concentration ranging from 1-10 mM each NTP, the DNA template at a concentration ranging from 0.01-0.5 mg/ml, and the SP6 RNA polymerase at a concentration ranging from 0.01-0.1 mg/ml. For example, the reaction mixture may comprise NTPs at a concentration of 5 mM each NTP, the DNA template at a concentration of 0.1 mg/ml, and the SP6 RNA polymerase at a concentration of 0.05 mg/ml. The NTPs may be naturally-occurring NTPs, or may comprise modified NTPs.

In some embodiments, the mRNA may be synthesized at a temperature ranging from 37-56° C., e.g. at 50-52° C.

In some embodiments, the method for the production of mRNA may result in at least 80%, at least 85%, at least 90%, at least 95% of the mRNA transcripts terminating at the termination signal. The site of termination may be determined by (i) digestion of the mRNA to produce 3′ end fragments less than 100 nucleotides in size, and (ii) analysis of said 3′ end fragments by liquid chromatography. The site of termination may be determined by RNA sequencing.

In some embodiments, the RNA polymerase is a T7 RNA polymerase and wherein the mRNA transcripts are substantially free of RNA duplexes. The mRNA transcripts may contain undetectable levels of RNA duplexes relative to a control. The RNA duplexes may be detected with an antibody that specifically binds to dsRNA.

Any aspect or embodiment described herein can be combined with any other aspect or embodiment as disclosed herein. While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

Other features and advantages of the invention will be apparent from the Drawings and the following Detailed Description, including the Examples, and the claims.

### BRIEF DESCRIPTION OF THE DRAWINGS

The above and further features will be more clearly appreciated from the following detailed description when taken in conjunction with the accompanying drawings. The drawings however are for illustration purposes only; not for limitation.

FIG. 1, section I is an electropherogram showing the capillary electrophoresis profile of mRNA-1 synthesized with SP6 RNA polymerase.

FIG. 2 is a digital gel image generated from the quantitative analysis of the total RNA by capillary electrophoresis for mRNA-1 and for variants of mRNA-1 with point mutations in the TATCTGTT termination signal sequence, synthesized with SP6 RNA polymerase.

FIG. 3 is an image of a dot blot showing the amount of dsRNA detected in mRNA samples prepared with either SP6 RNA polymerase or T7 RNA polymerase. The presence of dsRNA was determine with the murine monoclonal antibody J2, using a horse radish peroxide-conjugated anti-mouse IgG antibody for detection. Any dsRNA potentially present in the samples prepared with SP6 RNA polymerase was below the lower limit of detection (LLOD). The amount of dsRNA in samples prepared with T7 RNA polymerase exceeded 25 ng.

FIG. 4 provides the results of the analysis of the 3′ ends of SP6 mRNA transcripts. mRNA transcribed by SP6 RNA polymerase was digested using RNaseH and the 3′ end digestion products were analyzed by liquid chromatography mass spectrometry (LC/MS) (FIG. 4A) and the fragment was identified based on its size as determined by mass spectrometry (FIG. 4B).

FIG. 5 compares non-templated elongation of SP6 RNA polymerase (top panels) and T7 RNA polymerase (bottom panels) mRNA transcripts. The number of extra nucleotides added to the 3′ end of mRNA transcripts following templated transcription was determined by LC/MS (FIG. 5A) and by RNA sequencing (FIG. 5B).

FIG. 6 provides electropherograms showing the capillary electrophoresis profile (section I) for mRNA-12 synthesized with SP6 RNA polymerase from linearized plasmids. The plasmid was either unmodified (FIG. 6A), or modified by the addition of one (FIG. 6B) or two (FIG. 6C) rrnB termination t1 signals at the 3′ end of the DNA sequence encoding the mRNA transcript.

FIG. 7 provides electropherograms showing the capillary electrophoresis profile (section I) for mRNA-12 synthesized with SP6 RNA polymerase from supercoiled (non-linearized) plasmids. The plasmid was either unmodified (FIG. 7A), or modified by the addition of one (FIG. 7B) or two (FIG. 7C) rrnB termination t1 signals at the 3′ end of the DNA sequence encoding the mRNA transcript.

FIG. 8 provides electropherograms showing the capillary electrophoresis profile (section I) for mRNA-12 synthesized with SP6 RNA polymerase from supercoiled (non-linearized) plasmids at 37° C. (FIG. 8A) or 50° C. (FIG. 8B). The plasmid was modified by the addition of two rrnB termination t1 signals at the 3′ end of the DNA sequence encoding the mRNA transcript.

FIG. 9 provides electropherograms showing the capillary electrophoresis profile generated for mRNA-12 synthesized with SP6 RNA polymerase from supercoiled (non-linearized) plasmids at 37° C. (FIG. 9A, 9C, 9E, 9G) or 50° C. (FIG. 9B, 9D, 9F, 9H). The plasmid was either unmodified (FIG. 9A, 9B), or modified by the addition of one (FIG. 9C, 9D), two (FIG. 9E, 9F) or three (FIG. 9G, 9H) rrnB termination t1 signals at the 3′ end of the DNA sequence encoding the mRNA transcript.

FIG. 10 compares levels of protein expressed from mRNA-12 transcribed from linearized unmodified plasmid (containing no termination sequence) and from mRNA-12 transcribed from a supercoiled plasmid modified by the addition of three rrnB termination t1 signals at the 3′ end of the DNA sequence encoding the mRNA transcript.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

Unless specifically stated or evident from context, as used herein, the term “about” is understood as within a range of normal tolerance in the art, for example within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflects normal fluctuations that can be appreciated by a skilled artisan.

As used herein, term “abortive transcript” or “pre-aborted transcript” or the like is any transcript that is shorter than a full-length mRNA molecule encoded by the DNA template that results from the premature release of RNA polymerase from the template DNA in a sequence-independent manner. In some embodiments, an abortive transcript may be less than 90% of the length of the full-length mRNA molecule that is transcribed from the target DNA molecule, e.g., less than 80%, 70%, 60%, 50%, 40%, 30%, 20%, 10%, 5%, 1% of the length of the full-length mRNA molecule.

As used herein, the term “batch” refers to a quantity or amount of mRNA synthesized at one time, e.g., produced according to a single manufacturing order during the same cycle of manufacture. A batch may refer to an amount of mRNA synthesized in one reaction that occurs via a single aliquot of enzyme and/or a single aliquot of DNA template for continuous synthesis under one set of conditions. In some embodiments, a batch would include the mRNA produced from a reaction in which not all reagents and/or components are supplemented and/or replenished as the reaction progresses. The term “batch” would not mean mRNA synthesized at different times that are combined to achieve the desired amount.

As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. Such modifications to the naturally-occurring or wild-type nucleic acid may be done to achieve the highest possible G/C content, to adjust codon usage to avoid rare or rate-limiting codons, to remove destabilizing nucleic acid sequences or motifs and/or to eliminate pause sites or terminator signals.

As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

As used herein, the terms “drug”, “medication”, “therapeutic”, “active agent”, “therapeutic compound”, “composition”, or “compound” are used interchangeably and refer to any chemical entity, pharmaceutical, drug, biological, botanical, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound”, “drug”, or “medication” refers to a therapeutic compound that has been shown (e.g., through animal trials or prior experience with administration to humans) to be effective in such treatment. A “therapeutic regimen” relates to a treatment comprising a “drug”, “medication”, “therapeutic”, “active agent”, “therapeutic compound”, “composition”, or “compound” as disclosed herein and/or a treatment comprising behavioral modification by the subject and/or a treatment comprising a surgical means.

As used herein, the term “encapsulation,” or grammatical equivalent, refers to the process of confining an mRNA molecule within a nanoparticle. The process of incorporation of a desired mRNA into a nanoparticle is often referred to as “loading”. Exemplary methods are described in Lasic, et al., FEBS Lett., 312: 255-258, 1992, which is incorporated herein by reference. The nanoparticle-incorporated nucleic acids may be completely or partially located in the interior space of the nanoparticle, within the bilayer membrane (for liposomal nanoparticles), or associated with the exterior surface of the nanoparticle.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein. In this application, the terms “expression” and “production,” and grammatical equivalent, are used inter-changeably.

As used herein, “full-length mRNA” is as characterized when using a specific assay, e.g., gel electrophoresis and detection using UV and UV absorption spectroscopy with separation by capillary electrophoresis. The length of an mRNA molecule that encodes a full-length polypeptide is at least 50% of the length of a full-length mRNA molecule that is transcribed from the target DNA, e.g., at least 60%, 70%, 80%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, 99.9% of the length of a full-length mRNA molecule that is transcribed from the target DNA.

As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

As used herein, the term “impurities” refers to substances inside a confined amount of liquid, gas, or solid, which differ from the chemical composition of the target material or compound. Impurities are also referred to as contaminants.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man.

As used herein, the term “messenger RNA (mRNA)” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

mRNA is typically thought of as the type of RNA that carries information from DNA to the ribosome. The existence of mRNA is usually very brief and includes processing and translation, followed by degradation. Typically, in eukaryotic organisms, mRNA processing comprises the addition of a “cap” on the N-terminal (5′) end, and a “tail” on the C-terminal (3′) end. A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. The tail is typically a polyadenylation event whereby poly A moiety is added to the 3′ end of the mRNA molecule. The presence of this “tail” serves to protect the mRNA from exonuclease degradation. Messenger RNA typically is translated by the ribosomes into a series of amino acids that make up a protein.

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. A nucleic acid sequence is presented in the 5′ to 3′ direction unless otherwise indicated.

As used herein, the term “premature termination” refers to the termination of transcription before the full length of the DNA template has been transcribed. Premature termination is caused by the presence of a termination signal within the DNA template and results in mRNA transcripts that are shorter than the full length mRNA (“prematurely terminated transcripts” or “truncated mRNA transcripts”). Examples of a termination signal include the E. coli rrnB terminator t1 signal (consensus sequence: ATCTGTT) and variants thereof, as described herein.

As used herein, the term “runoff transcription” refers to non-templated addition of nucleic acids at the end of mRNA transcripts. As described herein, RNA polymerases continue to elongate mRNA transcripts in a non-template-mediated fashion after encountering a transcription termination signal. The added sequences are referred to herein as “runoff” or “runoff sequences”. In some embodiments, runoff sequences may be able to self-anneal or anneal with portions of the templated mRNA transcript to form double-stranded or duplex RNA.

As used herein, the term “shortmer” is used to specifically refer to prematurely aborted short mRNA oligonucleotide, also called short abortive RNA transcripts, which are products of incomplete mRNA transcription during in vitro transcription reactions. Shortmers, prematurely aborted mRNA, pre-abortive mRNA, or short abortive mRNA transcripts are used interchangeably in the specification.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “template DNA” (or “DNA template”) typically relates to a DNA molecule comprising a nucleic acid sequence encoding the mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 and SP6 RNA polymerases, which is operably linked to the DNA sequence encoding a desired mRNA transcript. Furthermore the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

Unless otherwise defined, all technical and scientific terms used herein have the same meaning as commonly understood by one of ordinary skill in the art to which this application belongs and as commonly used in the art to which this application belongs; such art is incorporated by reference in its entirety. In the case of conflict, the present Specification, including definitions, will control.

### DETAILED DESCRIPTION OF THE INVENTION

The inadvertent presence of a termination signal including the consensus motif TATCTGTT in a DNA template sequence can result in the premature termination of in vitro transcription by SP6 and T7 RNA polymerases, leading to a heterogeneous population of mRNA transcripts in which the yield of the desired full-length mRNA transcript is significantly reduced. The inventors identified that a single point mutations at position 1, 6 or 8 of the consensus termination signal TATCTGTT is sufficient to prevent premature termination of in vitro transcription. The inventors also discovered that such variants of the previously identified consensus motif TATCTGTT are frequently present in codon-optimized DNA template sequences for use in in vitro transcription. Furthermore, previous work suggested that a T-rich sequence immediately 3′ of the consensus motif TATCTGTT is required for termination of transcription (Kwon & Kang 1999, The Journal of Biological Chemistry, 274:41, pp 29149-29155), but the inventors demonstrated that this not an essential element of the termination signal. The inventors' discovery makes it possible to screen for the termination signals and to effectively remove them from such DNA template sequences.

Accordingly, in one aspect, the invention is directed to a method for preparing an optimized DNA sequence encoding a protein as a template for in vitro transcription, said method comprising: (a) providing a DNA sequence that comprises a protein coding sequence; (b) determining the presence of a termination signal in the DNA sequence, wherein the termination signal has the following nucleic acid sequence: 5′-X₁ATCTX₂TX₃-3′ (SEQ ID NO: 1), wherein X₁, X₂ and X₃ are independently selected from A, C, T or G; and (c) if one or more termination signal is present, modifying the DNA sequence by replacing one or more nucleic acids at any one of position 2, 3, 4, 5 and 7 of said termination signal(s) with any one of the other three nucleic acids to generate the optimized DNA sequence, wherein, if required, the one or more replacement nucleic acids are selected to preserve the amino acid sequence of the protein encoded by the protein coding sequence.

SP6 RNA Polymerase synthesizes mRNA with significantly reduced abortive transcripts (so called “shortmers”) as compared to T7 RNA polymerase and therefore is uniquely suitable for large-scale in vitro synthesis of mRNA (see WO 2018/157153). In addition, the inventors demonstrate herein that, unlike T7 RNA polymerase, the mRNA transcripts synthesized by SP6 RNA polymerase do not form intra- or intermolecular duplexes and are therefore essentially free of duplex mRNA.

The inventors found that non-templated elongation of mRNA transcripts (“runoff” transcription) occurred during in vitro synthesizes when either SP6 RNA polymerase or T7 RNA polymerase was used. The presence of “runoff” sequences at the end of mRNA transcripts can be problematic for various reasons. For example, it increases the heterogeneity of the resulting mRNA preparation and therefore makes quality control more difficult, e.g., due to batch-to-batch variations. The “runoff” may also introduce unwanted elements relevant to mRNA processing and stability into the mRNA transcript. In addition, at least with respect to in vitro transcription processes that employ T7 RNA polymerases, “runoff” transcription results in the formation of RNA duplexes. In order to improve existing methods for the production of mRNA by in vitro synthesis, the invention provides methods and DNA sequences in which one or more termination signals are added at the 3′ end of the DNA template to prevent the non-templated elongation of mRNA transcripts. The inventors surprisingly found that the addition of one or more termination signals can be so effective in terminating transcription of the accordingly modified DNA template by an RNA polymerase that it is no longer necessary to linearize the plasmid comprising the DNA template prior to in vitro transcription. Removal of the linearization step, which typically involves incubation with a restriction enzyme, can result in considerable cost savings in the production of mRNA, in particular when done at a large scale to manufacture a drug product. In WO 2017/009376 and WO 2012/170443 a circular plasmid was used as a template for production of RNA by in vitro synthesis. However, the DNA template sequences included both sequences encoding a self-cleaving ribozyme and sequences encoding multiple termination signals. The inventors have demonstrated for the first time that over 90% termination efficiency can be achieved during mRNA synthesis by in vitro transcription from a circular DNA template by the addition of terminator sequences only.

Accordingly, in a further aspect, the invention provides a method for preparing an optimized DNA sequence encoding a protein as a template for in vitro transcription, said method comprising: (a) providing a DNA sequence encoding a protein; and (b) adding one or more termination signals at the 3′ end of the DNA sequence to provide the optimized DNA sequence, wherein the one or more termination signal(s) comprises the following nucleic acid sequence: 5′-X₁ATCTX₂TX₃-3′ (SEQ ID NO: 1), wherein X1, X₂ and X₃ are independently selected from A, C, T or G. The invention also provides a DNA sequence for use in in vitro transcription, comprising in 5′ to 3′ order: (a) a 5′UTR; (b) a protein coding sequence; (c) a 3′ UTR; (d) optionally a nucleic acid sequence encoding a polyA tail; and (e) a termination signal; wherein the termination signal comprises the following nucleic acid sequence: 5′-X₁ATCTX₂TX₃-3′ (SEQ ID NO: 1), wherein X₁, X₂ and X₃ are independently selected from A, C, T or G. Moreover, the invention provides nucleic acid vectors that comprise the DNA sequence, typically operably linked to an RNA polymerase promotor, and the use of these nucleic acid vectors in a method for the production of mRNA, wherein an RNA polymerase transcribes the DNA sequence into mRNA transcripts.

Various aspects of the invention are described in detail in the following sections. The use of sections is not meant to limit the invention. Each section can apply to any aspect of the invention.

### DNA Template

Various nucleic acid templates may be used in the present invention. Typically, DNA templates which are either entirely double-stranded or mostly single-stranded with a double-stranded SP6 promoter sequence can be used.

In some embodiments, the synthesized optimized DNA sequence is inserted in a nucleic acid vector for use in in vitro transcription. In some embodiments, the nucleic acid vector is a plasmid. The term ‘plasmid’ or ‘plasmid nucleic acid vector’ refers to a circular nucleic acid molecule, preferably to an artificial nucleic acid molecule. A plasmid DNA in the context of the present invention is suitable for incorporating or harboring a desired nucleic acid sequence, such as a nucleic acid sequence comprising a sequence encoding an RNA and/or an open reading frame encoding at least one protein, polypeptide or peptide. Such plasmid DNA constructs/vectors may be expression vectors, cloning vectors, transfer vectors, etc. The plasmid DNA typically comprises a sequence corresponding (coding for) a desired mRNA transcript, or a part thereof, such as a sequence corresponding to the open reading frame and the 5′- and/or 3′UTR of an mRNA. In some embodiments, the sequence corresponding to the desired mRNA transcript may also encode a polyA-tail after the 3′ UTR so that the polyA-tail is included with the mRNA transcript. More typically in the context of the present invention, the sequence corresponding to the desired mRNA transcript consists of the 5′/3′ UTRs and the open reading frame. In the latter embodiment of the invention, the mRNA transcript synthesized from the DNA plasmid during in vitro transcription does not contain a polyA tail, and post-synthesis processing of the mRNA transcript is required in order to add a polyA tail.

An expression vector may be used for production of expression products such as RNA, e.g. mRNA in a process called RNA in vitro transcription. For example, an expression vector may comprise sequences needed for RNA in vitro transcription of a sequence stretch of the vector, such as a promoter sequence, e.g., an RNA polymerase promoter sequence, such as T3, T7 or SP6 RNA polymerase promotor sequences.

A cloning vector is typically a vector that contains a cloning site, which may be used to incorporate (insert) nucleic acid sequences into the vector. A cloning vector may be, e.g., a plasmid vector or a bacteriophage vector. A transfer vector may be a vector, which is suitable for transferring nucleic acid molecules into cells or organisms, for example, viral vectors. A plasmid DNA vector suitable for use with the present invention typically comprises a multiple cloning site, an RNA polymerase promoter sequence, optionally a selection marker, such as an antibiotic resistance factor, and a sequence suitable for multiplication of the vector, such as an origin of replication. Particularly suitable are plasmid DNA vectors, or expression vectors, comprising promoters for DNA-dependent RNA polymerases such as T3, T7 and SP6. Suitable plasmids for practicing the invention include, e.g., pUC19 and pBR322.

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription with SP6/T7 RNA polymerase, provided that they contain a double-stranded SP6 promoter upstream (and in the correct orientation) of the DNA sequence to be transcribed, or with T7 RNA polymerase, provided that they contain a double-stranded T7 promoter upstream (and in the correct orientation) of the DNA sequence to be transcribed.

In some embodiments, the linearized DNA template has a blunt-end.

In a particular embodiment of the invention, the plasmid DNA does not require linearization for in vitro transcription. Specifically, the invention makes it possible for the first time to produce mRNA transcripts from circular nucleic acid vectors such as plasmid DNA (which is typically supercoiled) using a SP6/T7 RNA polymerase for in vitro transcription.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, and citric acid cycle enzymes) to increase the stability of the sense mRNA molecule. For example, a 5′ UTR sequence may include a partial sequence of a CMV immediate-early 1 (IEl) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

Sequence Optimization

An aspect of the invention relates to removal of terminator sequences within a DNA template to prepare an optimized DNA sequence. The method comprises, inter alia, the steps of determining the presence of a termination signal in the DNA sequence and, if one or more termination signals are present, modifying the DNA sequence by replacing one or more nucleic acids at any one of position 2, 3, 4, 5 and 7 of said termination signal(s) with any one of the other three nucleic acids to generate the optimized DNA sequence, wherein, if required, the one or more replacement nucleic acids are selected to preserve the amino acid sequence of the protein encoded by the protein coding sequence. The termination signal may be detected anywhere in the DNA sequence (e.g., within the region encoding the protein coding sequence, within the region encoding the 5′ untranslated region and/or within the region encoding the 3′ untranslated region). The above steps may be carried out by a computer. Computer programs suitable for detecting the presence of a specified nucleic acid sequence (e.g., the termination signal of the invention) within a DNA sequence and identifying nucleic acid substitutions that preserve the amino acid sequence of the protein encoded by the protein coding sequence are well-known in the art.

The DNA sequence to be transcribed may be further optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., 1RES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which is described in US 20110081708, the contents of which are incorporated herein by reference in its entirety.

In some embodiments, a codon optimization algorithm is used to modify the DNA sequence to facilitate more efficient transcription and/or translation. In some embodiments, a codon optimization algorithm determines the presence of a termination signal in the DNA sequence. In some embodiments, a codon optimization algorithm modifies the DNA sequence by replacing one or more nucleic acids, and may, if required, select one or more replacement nucleic acids to preserve the amino acid sequence of the protein encoded by the protein coding sequence.

In a particular embodiment, a codon optimization algorithm determines the presence of a termination signal in the DNA sequence, wherein the termination signal has the following nucleic acid sequence: 5′-X₁ATCTX₂TX₃-3′ (SEQ ID NO: 1), wherein X₁, X₂ and X₃ are independently selected from A, C, T or G; and if one or more termination signal is present, modifies the DNA sequence by replacing one or more nucleic acids at any one of position 2, 3, 4, 5 and 7 of said termination signal(s) with any one of the other three nucleic acids to generate the optimized DNA sequence, wherein, if required, the one or more replacement nucleic acids are selected to preserve the amino acid sequence of the protein encoded by the protein coding sequence.

A codon optimization algorithm generates sequences by maximizing the codon adaptation index (CAI). CAI is a numerical score of codon usage bias for measuring a sequence's deviation from a reference set of genes. In some embodiments, the genes of the reference set are mammalian genes. In a particular embodiment, the genes of the reference set are human genes. The CAI is typically calculated on the basis of the frequency of use of all codons in a protein codon sequence of interest. In a first step, the codon optimization algorithm reiteratively modifies an input protein codon sequence to achieve a first output sequence with an optimal CAI. In a second step, the first output sequence is analyzed for the presence of sequence elements that are known to negatively affect gene expression at the transcription or translation level. This includes the termination signal described herein. If such sequence elements are identified, the codon optimization algorithm modifies the first output sequence to remove them, thereby generating a second output sequence. In the same or a subsequent step, the first or second output sequence is also analyzed for one or more of the following parameters: GC content, stable free energy of the encoded mRNA transcript, and the presence of out-of-frame start codons. If necessary, the first or second output sequence is modified to optimize one or more of these parameters. For example, any out-of-frame start codons may be removed by appropriate codon substitutions. Output sequences with a lower GC content typically have more negative value of free energy than output sequences with a higher GC content. The most negative value of free energy is thought to result in the most structured and accordingly the most stable mRNA transcript. Accordingly, in some embodiment, the algorithm increases the GC content of the first or second output sequence by further codon substitutions.

### Targeted Insertion of Termination Signals

Another aspect of the invention relates to the inclusion of one or more termination signal(s) at the 3′ end of a DNA sequence encoding a protein of interest (e.g., a therapeutic protein) to prepare an optimized DNA sequence as a template for in vitro transcription. Targeted insertion of one or more termination signal (e.g., two or three termination signals) at the 3′ end of the DNA sequence that encodes the mRNA transcript can obviate the need for linearization of a plasmid encoding the template prior to in vitro transcription. Accordingly, in one aspect, the invention relates to a DNA sequence for use in in vitro transcription, comprising in 5′ to 3′ order:
a 5′UTR;
a protein coding sequence;
a 3′UTR;
optionally a nucleic acid sequence encoding a polyA tail; and
a termination signal.

In accordance with the invention, the termination signal comprises the following nucleic acid sequence: 5′-X₁ATCTX₂TX₃-3′ (SEQ ID NO: 1), wherein X1, X₂ and X₃ are independently selected from A, C, T or G. In one embodiment, the termination signal comprises the nucleic acid sequence 5′-X₁ATCTGTT-3′ (SEQ ID NO: 2). X₁ may be T or C. A suitable termination may be selected from 5′-TTTTATCTGTTTTTTT-3′ (SEQ ID NO: 3), 5′-TTTTATCTGTTTTTTTTT-3′ (SEQ ID NO: 4), 5′-CGTTTTATCTGTTTTTTT-3′ (SEQ ID NO: 5), 5′-CGTTCCATCTGTTTTTTT-3′ (SEQ ID NO: 6), 5′-CGTTTTATCTGTTTGTTT-3′ (SEQ ID NO: 7), 5′-CGTTTTATCTGTTTGTTT-3′ (SEQ ID NO: 8), or 5′-CGTTTTATCTGTTGTTTT-3′ (SEQ ID NO: 9).

Typically, the DNA sequence comprises more than one termination signal, e.g., two or more, three or more, or four or more. The inventors have shown that for effective termination to occur, the termination signals can be separated by 10 base pairs or fewer, e.g., separated by 5-10 base pairs. In some embodiments, the DNA sequence comprises two termination signals (e.g., 5′-X₁ATCTX₂TX₃-3′ (SEQ ID NO: 1), wherein X₁, X₂ and X₃ are independently selected from A, C, T or G) within a nucleotide sequence of 30 nucleotides in length. Accordingly, in some embodiments, a DNA sequence for use with the invention comprises the following sequence at its 3′ end: 5′-X₁ATCTX₂TX₃-(Z_{N})—X₄ATCTX₅TX₆-3′ (SEQ ID NO: 10), wherein X₁, X₂, X₃, X₄, X₅ and X₆ are independently selected from A, C, T or G and Z_{N} represents a spacer sequence of N nucleotides, each of which are independently selected from A, C, T or G, and wherein N is 10 or fewer. For example, N can be 5, 6, 7, 8, 9 or 10. Z can be T. In some embodiments, the DNA sequence comprises the following sequence: TTTTATCTGTTTTTTTTTTTTTATCTGTTTTTTTTT (SEQ ID NO: 12). In other embodiments, the DNA sequence comprises three termination signals (e.g., 5′-X₁ATCTX₂TX₃-3′ (SEQ ID NO: 1), wherein X1, X₂ and X₃ are independently selected from A, C, T or G) within a nucleotide sequence of 50 nucleotides in length. Accordingly, in some embodiments, a DNA sequence for use with the invention comprises the following sequence at its 3′ end: 5′-X₁ATCTX₂TX₃-Z_{N})-X₄ATCTX₅TX₆-(Z_{M})-X₇ATCTX₈TX₉-3′ (SEQ ID NO: 11), wherein X1, X₂, X₃, X₄, X₅, X6, X₇, X₈ and X₉ are independently selected from A, C, T or G, Z_{N} represents a spacer sequence of N nucleotides, and Z_{M} represents a spacer sequence of M nucleotides, each of which are independently selected from A, C, T or G, and wherein N and/or M are independently 10 or fewer. For example, N can be 5, 6, 7, 8, 9 or 10. M can be 5, 6, 7, 8, 9 or 10. Z can be T. In a specific embodiments, the DNA sequence comprises the following sequence at its 3′ end:

(SEQ ID NO: 13)
TTTTATCTGTTTTTTTTTTTTTATCTGTTTTTTTTTTTTTATCTGTTTTT
TTTT.

As shown herein, having two termination signal in sequence at the 3′ end of the DNA sequence can result in effective termination of in vitro transcription. The examples of the present application further demonstrate that a yield of correctly terminated mRNA transcripts approaching 100% can be reached when more than two copies of a termination signal is present at the 3′ end of a DNA sequence encoding the mRNA transcript. In particular, adding three or more termination signals in sequence to the 3′ end of the DNA sequence can yield 100% termination. This observation has been made when in vitro transcription was performed at 37° C.

Moreover, the inventors have shown that minimal termination sequences of two or three (or more) terminator signals in sequence (e.g., TATCTGTT), spaced apart by 10 nucleotides (e.g., Ts) or less, are sufficient for effective termination of the in vitro transcription at the end of the DNA sequence. Accordingly, a DNA sequence for use with the invention does not comprise any further termination signals and/or sequences. DNA sequences with the minimal termination sequences of the invention can produce correctly-terminated mRNA transcripts without the need for a ribozyme sequence at the 3′ end or an alternative terminator signal. Accordingly, in some embodiments, the DNA sequence does not comprise a further sequence encoding a ribozyme at its 3′ end. In addition or alternatively, the DNA sequence does not comprise a class I termination signal. Indeed, no other termination signals in addition to the minimal terminator sequences disclosed here are required to effect termination of in vitro transcription.

In accordance with the invention, the termination signal is absent from the 5′ UTR, the protein coding sequence and the 3′ UTR of the DNA sequence to avoid premature termination of in vitro transcription before the RNA polymerase has reached the 3′ end of the DNA sequence.

Also provided herein is a method for preparing the DNA sequence described in the foregoing paragraphs. The method comprises: (a) providing a DNA sequence encoding a protein; and (b) adding one or more termination signals at the 3′ end of the DNA sequence to provide the DNA sequence, wherein the one or more termination signal(s) comprise the following nucleic acid sequence: 5′-X₁ATCTX₂TX₃-3′ (SEQ ID NO: 1), wherein X₁, X₂ and X₃ are independently selected from A, C, T or G. In some embodiments, the termination signal added at the 3′ end of the DNA sequence comprises the following sequence:

(SEQ ID NO: 14)
TTTTATCTGTTTTTTTTTT.

The examples of the present application demonstrate that the addition of two or more termination signals results in a reduction in undesired elongation of mRNA transcripts during in vitro transcription, both for linear and for super-coiled DNA templates. Therefore, in some embodiments, two or more, three or more, four or more termination signals are added to the 3′ end of the DNA sequence. In some embodiments, the termination sequence added to the 3′ end comprises or consists of two termination signals (e.g., 5′-X₁ATCTX₂TX₃-3′ (SEQ ID NO: 1), wherein X1, X₂ and X₃ are independently selected from A, C, T or G) within a nucleotide sequence of 30 nucleotides in length. In some embodiments, the termination sequence added to the 3′ end comprises or consists of the following sequence: 5′-X1ATCTX₂TX₃-(Z_{N})-X₄ATCTX₅TX₆-3′ (SEQ ID NO: 10), wherein X₁, X₂, X3, X₄, X₅, and X₆ are independently selected from A, C, T or G, Z_{N} represents a spacer sequence of N nucleotides, each of which are independently selected from A, C, T or G, and wherein N is 10 or fewer. In some embodiments, the termination sequence comprises or consists of the following sequence:

(SEQ ID NO: 12)
TTTTATCTGTTTTTTTTTTTTTATCTGTTTTTTTTT.

In some embodiments, the termination sequence added to the 3′ end comprises or consists of three termination signals (e.g., 5′-XATCTXTX-3′ (SEQ ID NO: 1), wherein X₁, X₂ and X3 are independently selected from A, C, T or G) within a nucleotide sequence of 50 nucleotides in length. In some embodiments, three termination signals are added to the 3′ end of the DNA sequence. In some embodiments, the termination sequence added to the 3′ end comprises or consist of the following sequence: 5′-X₁ATCTX₂TX₃-(Z_{N})—X₄ATCTX₅TX₆-(Z_{M})-X₇ATCTX₈TX₉-3′ (SEQ ID NO: 11), wherein X₁, X₂, X₃, X₄, X5, X6, X₇, X₈ and X₉ are independently selected from A, C, T or G, Z_{N} represents a spacer sequence of N nucleotides, and Z_{M} represents a spacer sequence of M nucleotides, each of which are independently selected from A, C, T or G, and wherein N and/or M are independently 10 or fewer. In some embodiments, the termination sequence comprises or consists of the following sequence:

(SEQ ID NO: 13)
TTTTATCTGTTTTTTTTTTTTTATCTGTTTTTTTTTTTTTATCTGTTTTT
TTTT.

### SP6 RNA Polymerase

SP6 RNA Polymerase is a DNA-dependent RNA polymerase with high sequence specificity for SP6 promoter sequences. Typically, this polymerase catalyzes the 5′->3′ in vitro synthesis of RNA on either single-stranded DNA or double-stranded DNA downstream from its promoter; it incorporates native ribonucleotides and/or modified ribonucleotides into the polymerized transcript.

The sequence for bacteriophage SP6 RNA polymerase was initially described (GenBank: Y00105.1) as having the following amino acid sequence:

(SEQ ID NO: 15)
MQDLHAIQLQLEEEMFNGGIRRFEADQQRQIAAGSESDTAWNRRLLSELI
APMAEGIQAYKEEYEGKKGRAPRALAFLQCVENEVAAYITMKVVMDMLNT
DATLQAIAMSVAERIEDQVRFSKLEGHAAKYFEKVKKSLKASRTKSYRHA
HNVAVVAEKSVAEKDADFDRWEAWPKETQLQIGTTLLEILEGSVFYNGEP
VFMRAMRTYGGKTIYYLQTSESVGQWISAFKEHVAQLSPAYAPCVIPPRP
WRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGR
ELKEMLSPEQWQQFINWKGECARLYTAETKRGSKSAAVVRMVGQARKYSA
FESIYFVYAMDSRSRVYVQSSTLSPQSNDLGKALLRFTEGRPVNGVEALK
WFCINGANLWGWDKKTFDVRVSNVLDEEFQDMCRDIAADPLTFTQWAKAD
APYEFLAWCFEYAQYLDLVDEGRADEFRTHLPVHQDGSCSGIQHYSAMLR
DEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLE
EKEAQKAVAEGRTANKVHPFEDDRQDYLTPGAAYNYMTALIWPSISEVVK
APIVAMKMIRQLARFAAKRNEGLMYTLPTGFILEQKIMATEMLRVRTCLM
GDIKMSLQVETDIVDEAAMMGAAAPNFVHGHDASHLILTVCELVDKGVTS
IAVIHDSFGTHADNTLTLRVALKGQMVAMYIDGNALQKLLEEHEVRWMVD
TGIEVPEQGEFDLNEIMDSEYVFA.

An SP6 RNA polymerase suitable for the present invention can be any enzyme having substantially the same polymerase activity as bacteriophage SP6 RNA polymerase. Thus, in some embodiments, an SP6 RNA polymerase suitable for the present invention may be modified from SEQ ID NO: 15. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, or 60% identical or homologous to SEQ ID NO: 15. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

In some embodiments, an SP6 RNA Polymerase is encoded by a gene having the following nucleotide sequence:

(SEQ ID NO: 16)
ATGCAAGATTTACACGCTATCCAGCTTCAATTAGAAGAAGAGATGTTTAA
TGGTGGCATTCGTCGCTTCGAAGCAGATCAACAACGCCAGATTGCAGCAG
GTAGCGAGAGCGACACAGCATGGAACCGCCGCCTGTTGTCAGAACTTATT
GCACCTATGGCTGAAGGCATTCAGGCTTATAAAGAAGAGTACGAAGGTAA
GAAAGGTCGTGCACCTCGCGCATTGGCTTTCTTACAATGTGTAGAAAATG
AAGTTGCAGCATACATCACTATGAAAGTTGTTATGGATATGCTGAATACG
GATGCTACCCTTCAGGCTATTGCAATGAGTGTAGCAGAACGCATTGAAGA
CCAAGTGCGCTTTTCTAAGCTAGAAGGTCACGCCGCTAAATACTTTGAGA
AGGTTAAGAAGTCACTCAAGGCTAGCCGTACTAAGTCATATCGTCACGCT
CATAACGTAGCTGTAGTTGCTGAAAAATCAGTTGCAGAAAAGGACGCGGA
CTTTGACCGTTGGGAGGCGTGGCCAAAAGAAACTCAATTGCAGATTGGTA
CTACCTTGCTTGAAATCTTAGAAGGTAGCGTTTTCTATAATGGTGAACCT
GTATTTATGCGTGCTATGCGCACTTATGGCGGAAAGACTATTTACTACTT
ACAAACTTCTGAAAGTGTAGGCCAGTGGATTAGCGCATTCAAAGAGCACG
TAGCGCAATTAAGCCCAGCTTATGCCCCTTGCGTAATCCCTCCTCGTCCT
TGGAGAACTCCATTTAATGGAGGGTTCCATACTGAGAAGGTAGCTAGCCG
TATCCGTCTTGTAAAAGGTAACCGTGAGCATGTACGCAAGTTGACTCAAA
AGCAAATGCCAAAGGTTTATAAGGCTATCAACGCATTACAAAATACACAA
TGGCAAATCAACAAGGATGTATTAGCAGTTATTGAAGAAGTAATCCGCTT
AGACCTTGGTTATGGTGTACCTTCCTTCAAGCCACTGATTGACAAGGAGA
ACAAGCCAGCTAACCCGGTACCTGTTGAATTCCAACACCTGCGCGGTCGT
GAACTGAAAGAGATGCTATCACCTGAGCAGTGGCAACAATTCATTAACTG
GAAAGGCGAATGCGCGCGCCTATATACCGCAGAAACTAAGCGCGGTTCAA
AGTCCGCCGCCGTTGTTCGCATGGTAGGACAGGCCCGTAAATATAGCGCC
TTTGAATCCATTTACTTCGTGTACGCAATGGATAGCCGCAGCCGTGTCTA
TGTGCAATCTAGCACGCTCTCTCCGCAGTCTAACGACTTAGGTAAGGCAT
TACTCCGCTTTACCGAGGGACGCCCTGTGAATGGCGTAGAAGCGCTTAAA
TGGTTCTGCATCAATGGTGCTAACCTTTGGGGATGGGACAAGAAAACTTT
TGATGTGCGCGTGTCTAACGTATTAGATGAGGAATTCCAAGATATGTGTC
GAGACATCGCCGCAGACCCTCTCACATTCACCCAATGGGCTAAAGCTGAT
GCACCTTATGAATTCCTCGCTTGGTGCTTTGAGTATGCTCAATACCTTGA
TTTGGTGGATGAAGGAAGGGCCGACGAATTCCGCACTCACCTACCAGTAC
ATCAGGACGGGTCTTGTTCAGGCATTCAGCACTATAGTGCTATGCTTCGC
GACGAAGTAGGGGCCAAAGCTGTTAACCTGAAACCCTCCGATGCACCGCA
GGATATCTATGGGGCGGTGGCGCAAGTGGTTATCAAGAAGAATGCGCTAT
ATATGGATGCGGACGATGCAACCACGTTTACTTCTGGTAGCGTCACGCTG
TCCGGTACAGAACTGCGAGCAATGGCTAGCGCATGGGATAGTATTGGTAT
TACCCGTAGCTTAACCAAAAAGCCCGTGATGACCTTGCCATATGGTTCTA
CTCGCTTAACTTGCCGTGAATCTGTGATTGATTACATCGTAGACTTAGAG
GAAAAAGAGGCGCAGAAGGCAGTAGCAGAAGGGCGGACGGCAAACAAGGT
ACATCCTTTTGAAGACGATCGTCAAGATTACTTGACTCCGGGCGCAGCTT
ACAACTACATGACGGCACTAATCTGGCCTTCTATTTCTGAAGTAGTTAAG
GCACCGATAGTAGCTATGAAGATGATACGCCAGCTTGCACGCTTTGCAGC
GAAACGTAATGAAGGCCTGATGTACACCCTGCCTACTGGCTTCATCTTAG
AACAGAAGATCATGGCAACCGAGATGCTACGCGTGCGTACCTGTCTGATG
GGTGATATCAAGATGTCCCTTCAGGTTGAAACGGATATCGTAGATGAAGC
CGCTATGATGGGAGCAGCAGCACCTAATTTCGTACACGGTCATGACGCAA
GTCACCTTATCCTTACCGTATGTGAATTGGTAGACAAGGGCGTAACTAGT
ATCGCTGTAATCCACGACTCTTTTGGTACTCATGCAGACAACACCCTCAC
TCTTAGAGTGGCACTTAAAGGGCAGATGGTTGCAATGTATATTGATGGTA
ATGCGCTTCAGAAACTACTGGAGGAGCATGAAGTGCGCTGGATGGTTGAT
ACAGGTATCGAAGTACCTGAGCAAGGGGAGTTCGACCTTAACGAAATCAT
GGATTCTGAATACGTATTTGCCTAA.

A suitable gene encoding the SP6 RNA polymerase suitable in the present may be about 99%, 98%, 97%, 96%, 95%, 94%, 93%, 92%, 91%, 90%, 89%, 88%, 87%, 86%, 85%, 84%, 83%, 82%, 81%, or 80% identical or homologous to SEQ ID NO: 16.

An SP6 RNA polymerase suitable for the invention may be a commercially-available product, e.g., from Ambion, New England Biolabs (NEB), Promega, and Roche. The SP6 may be ordered and/or custom designed from a commercial source or a non-commercial source according to the amino acid sequence of SEQ ID NO: 15 or a variant of SEQ ID NO: 15 as described herein. The SP6 RNA polymerase may be a standard-fidelity polymerase or may be a high-fidelity/high-efficiency/high-capacity which has been modified to promote RNA polymerase activities, e.g., mutations in the SP6 RNA polymerase gene or post-translational modifications of the SP6 RNA polymerase itself. Examples of such modified SP6 include SP6 RNA Polymerase-Plus™ from Ambion, HiScribe SP6 from NEB, and RiboMAX™ and Riboprobe® Systems from Promega.

In some embodiments, the SP6 RNA polymerase is thermostable. In a particular embodiment, the amino acid sequence of an SP6 RNA polymerase for use with the invention contains one or more mutations relative to a wild-type SP6 polymerase that render the enzyme active at temperatures ranging from 37° C. to 56° C. In some embodiment, an SP6 RNA polymerase for use with the invention functions at an optimal temperature of 50° C.-52° C. In other embodiment, an SP6 RNA polymerase for use with the invention has a half-life of at least 60 minutes at 50° C. For example, a particularly suitable SP6 RNA polymerase for use with the invention has a half-life of between 60 minutes and 120 minutes (e.g., between 70 minutes and 100 minutes, or 80 minutes to 90 minutes) at 50° C.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6) (SEQ ID NO: 38)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., Costa et al. Frontiers in Microbiology 5 (2014): 63 and in PCT/US16/57044, the contents of which are incorporated herein by reference in their entireties. In some embodiments, a His tag is located at SP6's N-terminus.

SP6 Promoter

Any promoter that can be recognized by an SP6 RNA polymerase may be used in the present invention. Typically, an SP6 promoter comprises 5′ ATTTAGGTGACACTATAG-3′ (SEQ ID NO: 17). Variants of the SP6 promoter have been discovered and/or created to optimize recognition and/or binding of SP6 to its promoter. Non-limiting variants include but are not limited to:

(SEQ ID NO: 18 to SEQ ID NO: 27)
5′-ATTTAGGGGACACTATAGAAGAG-3′;
5′-ATTTAGGGGACACTATAGAAGG-3′;
5′-ATTTAGGGGACACTATAGAAGGG-3′;
5′-ATTTAGGTGACACTATAGAA-3′;
5′-ATTTAGGTGACACTATAGAAGA-3′;
5′-ATTTAGGTGACACTATAGAAGAG-3′;
5′-ATTTAGGTGACACTATAGAAGG-3′;
5′-ATTTAGGTGACACTATAGAAGGG-3′;
5′-ATTTAGGTGACACTATAGAAGNG-3′;
and
5′-CATACGATTTAGGTGACACTATAG-′.

In addition, a suitable SP6 promoter for the present invention may be about 95%, 90%, 85%, 80%, 75%, or 70% identical or homologous to any one of SEQ ID NO: 18 to SEQ ID NO: 27. Moreover, an SP6 promoter suitable in the present invention may include one or more additional nucleotides 5′ and/or 3′ to any of the promoter sequences described herein.

RNA polymerase

T7 RNA Polymerase is a DNA-dependent RNA polymerase with high sequence specificity for T7 promoter sequences. Typically, this polymerase catalyzes the 5′->3′ in vitro synthesis of RNA on either single-stranded DNA or double-stranded DNA downstream from its promoter; it incorporates native ribonucleotides and/or modified ribonucleotides into the polymerized transcript.

In some embodiments, the T7 RNA polymerase is thermostable. In a particular embodiment, the amino acid sequence of a T7 RNA polymerase for use with the invention contains one or more mutations relative to a wild-type T7 polymerase that render the enzyme active at temperatures ranging from 37° C. to 56° C. An example for a suitable RNA polymerase is Hi-T7® RNA Polymerase from NEB. In some embodiment, a T7 RNA polymerase for use with the invention functions at an optimal temperature of 50° C.-52° C. In other embodiment, a T7 RNA polymerase for use with the invention has a half-life of at least 60 minutes at 50° C. For example, a particularly suitable T7 RNA polymerase for use with the invention has a half-life of between 60 minutes and 120 minutes (e.g., between 70 minutes and 100 minutes, or 80 minutes to 90 minutes) at 50° C.

### T7 Promotor

Any promoter that can be recognized by an T7 RNA polymerase may be used in the present invention. Typically, a T7 promoter comprises

(SEQ ID NO: 28)
5′-TAATACGACTCACTATAG-3′

mRNA Synthesis

mRNAs according to the present invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, a suitable template sequence is a DNA sequence encoding a protein, a polypeptide or a peptide. In some embodiments, a suitable template sequence is codon optimized for efficient expression in human cells. Codon optimization typically includes modifying a naturally-occurring or wild-type nucleic acid sequence encoding a peptide, polypeptide or protein to achieve the highest possible G/C content, to adjust codon usage to avoid rare or rate-limiting codons, to remove destabilizing nucleic acid sequences or motifs and/or to eliminate pause sites or terminator sequences without altering the amino acid sequence of the mRNA encoded peptide, polypeptide or protein. In some embodiments, a suitable protein-encoding sequence is naturally-occurring or a wild-type sequence. In some embodiments, a suitable protein-encoding sequence encodes a protein, a polypeptide or a peptide that contains one or more mutations in its amino acid sequence.

The methods disclosed herein can be used for the large-scale production of mRNA. In some embodiments, a method according to the invention synthesizes at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more mRNA in a single batch. In some embodiments, a method according to the invention synthesizes at least 1 kg, 10 kg or 100 kg in a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount. Generally, a reaction mixture includes RNA polymerase, a DNA template, and an RNA polymerase reaction buffer (which may include ribonucleotides or may require addition of ribonucleotides). The DNA template can be linear, although more typically in the context of the invention it will be circular.

According to the present invention, 1-100 mg of RNA polymerase is typically used per gram (g) of mRNA produced. In some embodiments, about 1-90 mg, 1-80 mg, 1-60 mg, 1-50 mg, 1-40 mg, 10-100 mg, 10-80 mg, 10-60 mg, 10-50 mg of RNA polymerase is used per gram of mRNA produced. In some embodiments, about 5-20 mg of RNA polymerase is used to produce about 1 gram of mRNA. In some embodiments, about 0.5 to 2 grams of RNA polymerase is used to produce about 100 grams of mRNA. In some embodiments, about 5 to 20 grams of RNA polymerase is used to about 1 kilogram of mRNA. In some embodiments, at least 5 mg of RNA polymerase is used to produce at least 1 gram of mRNA. In some embodiments, at least 500 mg of RNA polymerase is used to produce at least 100 grams of mRNA. In some embodiments, at least 5 grams of RNA polymerase is used to produce at least 1 kilogram of mRNA. In some embodiments, about 10 mg, 20 mg, 30 mg, 40 mg, 50 mg, 60 mg, 70 mg, 80 mg, 90 mg, or 100 mg of plasmid DNA is used per gram of mRNA produced. In some embodiments, about 10-30 mg of plasmid DNA is used to produce about 1 gram of mRNA. In some embodiments, about 1 to 3 grams of plasmid DNA is used to produce about 100 grams of mRNA. In some embodiments, about 10 to 30 grams of plasmid DNA is used to about 1 kilogram of mRNA. In some embodiments, at least 10 mg of plasmid DNA is used to produce at least 1 gram of mRNA. In some embodiments, at least 1 gram of plasmid DNA is used to produce at least 100 grams of mRNA. In some embodiments, at least 10 grams of plasmid DNA is used to produce at least 1 kilogram of mRNA.

In some embodiments, the concentration of the RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the RNA polymerase is from about 10 to 50 nM, 20 to 50 nM, or 30 to 50 nM. A concentration of 100 to 10000 Units/ml of the RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

In a particular embodiment, the concentration of each rNTP in a reaction mixture is optimized based on the frequency of each nucleic acid in the nucleic acid sequence that encodes a given mRNA transcript. Specifically, such a sequence-optimized reaction mixture comprises a ratio of each of the four rNTPs (e.g., ATP, GTP, CTP and UTP) that corresponds to the ratio of these four nucleic acids (A, G, C and U) in the mRNA transcript.

In some embodiments, a start nucleotide is added to the reaction mixture before the start of the in vitro transcription. A start nucleotide is a nucleotide which corresponds to the first nucleotide of the mRNA transcript (+1 position). The start nucleotide may be especially added to increase the initiation rate of the RNA polymerase. The start nucleotide can be a nucleoside monophosphate, a nucleoside diphosphate, a nucleoside triphosphate. The start nucleotide can be a mononucleotide, a dinucleotide or a trinucleotide. In embodiments where the first nucleotide of the mRNA transcript is a G, the start nucleotide is typically GTP or GMP. In a specific embodiment, the start nucleotide is a cap analog. The cap analog may be selected from the group consisting of G[5′]ppp[5′]G, m⁷G[5′]ppp[5′]G, m₃2,2,7G[5′]ppp[5′]G, m₂⁷′3′-°G[5′]ppp[5′]G (3′-ARCA), m₂^{7,2}′-°GpppG (2′-ARCA), m₂^{7,2}′_°GppspG D1 (0-S-ARCA D1) and m₂^{7,2}′-°GppspG D2 (0-S-ARCA D2).

In specific embodiments, the first nucleotide of the RNA transcript is G, the start nucleotide is a cap analog of G and the corresponding rNTP is GTP. In such embodiments, the cap analog is present in the reaction mixture in an excess in comparison to GTP. In some embodiments, the cap analog is added with an initial concentration in the range of about 1 mM to about 20 mM, about 1 mM to about 17.5 mM, about 1 mM to about 15 mM, about 1 mM to about 12.5 mM, about 1 mM to about 10 mM, about 1 mM to about 7.5 mM, about 1 mM to about 5 mM or about 1 mM to about 2.5 mM.

More typically in the context of the present invention, a cap structure such as a cap analog is added to the mRNA transcripts obtained during in vitro transcription only after the mRNA transcripts have been synthesized, e.g., in a post-synthesis processing step. Typically, in such embodiments, the mRNA transcripts are first purified (e.g., by tangential flow filtration) before a cap structure is added.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

DNA template (e.g., as described above and in an amount/concentration sufficient to provide a desired amount of RNA), the RNA polymerase reaction buffer, and RNA polymerase are combined to form the reaction mixture. The reaction mixture is incubated at between about 37° C. and about 56° C. for thirty minutes to six hours, e.g., about sixty to about ninety minutes. In some embodiments, incubation takes place at about 37° C. to about 42° C. In other embodiment, incubation takes place at about 43° C. to about 56° C., e.g. at about 50° C. to about 52° C. As demonstrated herein, the yield of accurately terminated mRNA transcripts obtained in an in vitro transcription reaction can be increased significantly by including one or more termination signals described herein at the end of a DNA sequence encoding an mRNA transcript of interest and performing the reaction with a template including the DNA sequences at a temperature between about 50° C. to about 52° C.

In some embodiments, about 5 mM NTPs, about 0.05 mg/mL RNA polymerase, and about 0.1 mg/ml DNA template in a suitable RNA polymerase reaction buffer (final reaction mixture pH of about 7.5) is incubated at about 37° C. to about 42° C. for sixty to ninety minutes. In other embodiments, about 5 mM NTPs, about 0.05 mg/mL RNA polymerase, and about 0.1 mg/ml DNA template in a suitable RNA polymerase reaction buffer (final reaction mixture pH of about 7.5) is incubated at about 50° C. to about 52° C. for sixty to ninety minutes.

In some embodiments, a reaction mixture contains a double stranded DNA template with an RNA polymerase-specific promoter, RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at lO× is 100 mM Tris-HCl, 5 mM MgCl₂ and 25 mM CaCl₂), pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and RNA polymerase at a concentration ranging from 0.01-0.1 mg/ml, e.g., the reaction mixture comprises NTPs at a concentration of 5 mM, the DNA template at a concentration of 0.1 mg/ml, and the RNA polymerase at a concentration of 0.05 mg/ml.

### Nucleotides

Various naturally-occurring or modified nucleosides may be used to produce mRNA according to the present invention. In some embodiments, an mRNA transcript in accordance with the invention is synthesized with natural nucleosides (i.e., adenosine, guanosine, cytidine, uridine). In other embodiments, an mRNA transcript in accordance with the invention is synthesized with natural nucleosides (e.g., adenosine, guanosine, cytidine, uridine) and one or of the following: nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N 1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“Ψ/U”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

Synthesized mRNA

The present invention provides high quality in vitro synthesized mRNA. For example, the present invention provides uniformity/homogeneity of synthesized mRNA. In particular, a composition of the present invention includes a plurality of mRNA molecules which are substantially full-length. For example, at least 81%, 82%, 83%, 84%, 85%, 86%, 87%, 88%, 89%, 90%, 91%, 92%, 93%, 94%, 95%, 96%, 97%, 98%, 99%, of the mRNA molecules are full-length mRNA molecules. Such a composition is said to be “enriched” for full-length mRNA molecules. In some embodiments, mRNA synthesized according to the present invention is substantially full-length. A composition of the present invention has a greater percentage of full-length mRNA molecules than a composition that is produced by a prior art process, i.e., a process that does include the use of optimized DNA sequence in accordance with the invention.

In some embodiments of the present invention, a composition or a batch is prepared without a step of specifically removing mRNA molecules that are not full-length mRNA molecules (i.e., abortive or aborted transcripts, or prematurely terminated transcripts).

In some embodiments, the mRNA molecules synthesized by the present invention are greater than 500, 600, 700, 800, 900, 1000, 2000, 3000, 4000, 5000, 10,000, or more nucleotides in length; also included in the present invention is mRNA having any length in between.

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to m7G(5′)ppp(5′)(2′OMeG), m7G(5′)ppp(5′)(2′OMeA), m7(3′OMeG)(5′)ppp(5′)(2′OMeG), m7(3′OMeG)(5′)ppp(5′)(2′OMeA), m7G(5′)ppp (5′(A,G(5′)ppp(5′)A and G(5′)ppp(5′)G. In a specific embodiment, the cap structure is m7G(5′)ppp(5′)(2′OMeG). Additional cap structures are described in published US Application No. US 2016/0032356 and U.S. Provisional Application 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly-A or poly-C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly(A) and poly(C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

Purification of mRNA

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol:chloroform:isoamyl alcohol solution, well known to one of skill in the art. In some embodiments, the mRNA is purified using Tangential Flow Filtration. Suitable purification methods include those described in US 2016/0040154, US 2015/0376220, PCT application PCT/US18/19978 entitled “METHODS FOR PURIFICATION OF MESSENGER RNA” filed on Feb. 27, 2018, and PCT application PCT/US 18/19954 entitled “METHODS FOR PURIFICATION OF MESSENGER RNA” filed on Feb. 27, 2018, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention.

In some embodiments, the mRNA is purified before capping and/or tailing. In some embodiments, the mRNA is purified before capping. In some embodiments, the mRNA is purified before tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF). In some embodiments, the mRNA may be subjected to further purification comprising dialysis, diafiltration and/or ultrafiltration.

In some embodiments, the mRNA is purified either before or after or both before and after capping and tailing by chromatography.

Precipitation of mRNA

mRNA in an impure preparation, such as an in vitro synthesis reaction mixture may be precipitated using a buffer and suitable conditions as described in U.S. Provisional Application No. 62/757,612 filed on Nov. 8, 2018, or in U.S. Provisional Application No. 62/891,781 filed on Aug. 26, 2019, and may be used to practice the present invention followed by various methods of purification known in the art. As used herein, the term “precipitation” (or any grammatical equivalent thereof) refers to the formation of an insoluble substance (e.g., solid) in a solution. When used in connection with mRNA, the term “precipitation” refers to the formation of insoluble or solid form of mRNA in a liquid.

Typically, mRNA precipitation involves a denaturing condition. As used herein, the term “denaturing condition” refers to any chemical or physical condition that can cause disruption of native confirmation of mRNA. Since the native conformation of a molecule is usually the most water soluble, disrupting the secondary and tertiary structures of a molecule may cause changes in solubility and may result in precipitation of mRNA from solution.

For example, a suitable method of precipitating mRNA from an impure preparation involves treating the impure preparation with a denaturing reagent such that the mRNA precipitates. Exemplary denaturing reagents suitable for the invention include, but are not limited to, lithium chloride, sodium chloride, potassium chloride, guanidinium chloride, guanidinium thiocyanate, guanidinium isothiocyanate, ammonium acetate and combinations thereof. Suitable reagent may be provided in a solid form or in a solution.

In some embodiments, a guanidinium salt is used in a denaturation buffer for precipitating mRNA. As non-limiting examples, guanidinium salts may include guanidinium chloride, guanidinium thiocyanate, or guanidinium isothiocyanate. Guanidinium thiocyanate (GCSN), also termed as guanidine thiocyanate, may be used to precipitate mRNA. Guanidinium salts such as guanidinium thiocyanate can be used at a concentration higher than is typically used for denaturing reactions, resulting in mRNA that is substantially free of protein contaminants. In some embodiments, a solution suitable for mRNA precipitation contains guanidine thiocyanate at a concentration greater than 4 M.

In a typical embodiment, an in vitro transcription reaction mixture containing the mRNA transcripts and/or the mixture resulting from the capping and/or tailing reaction, which comprises capped and tailed mRNA transcripts, is/are subjected to a purification process that comprises the addition of a denaturing agent such as guanidinium salts (e.g., guanidinium thiocyanate), followed by the addition of a precipitation agent (e.g., 100% ethanol) such that the mRNA precipitates from solution. The resulting mRNA suspension is added to a tangential flow filtration (TFF) column.

In addition to a denaturing reagent, a suitable solution for mRNA precipitation may include additionally a salt, a surfactant and/or a buffering agent. For example, a suitable solution may further include sodium lauryl sarcosyl and/or sodium citrate. In some embodiments, a buffer suitable for mRNA precipitation comprises about 5 mM sodium citrate. In some embodiments, a buffer suitable for mRNA precipitation comprises about 10 mM sodium citrate. In some embodiments, a buffer suitable for mRNA precipitation comprises about 20 mM sodium citrate. In some embodiments, a buffer suitable for mRNA precipitation comprises about 25 mM sodium citrate. In some embodiments, a buffer suitable for mRNA precipitation comprises about 30 mM sodium citrate. In some embodiments, a buffer suitable for mRNA precipitation comprises about 50 mM sodium citrate.

In some embodiments, a buffer suitable for mRNA precipitation comprises a surfactant, such as N-Lauryl Sarcosine (Sarcosyl). In some embodiments, a buffer suitable for mRNA precipitation comprises about 0.01% N-Lauryl Sarcosine. In some embodiments, a buffer suitable for mRNA precipitation comprises about 0.05% N-Lauryl Sarcosine. In some embodiments, a buffer suitable for mRNA precipitation comprises about 0.1% N-Lauryl Sarcosine. In some embodiments, a buffer suitable for mRNA precipitation comprises about 0.5% N-Lauryl Sarcosine. In some embodiments, a buffer suitable for mRNA precipitation comprises 1% N-Lauryl Sarcosine. In some embodiments, a buffer suitable for mRNA precipitation comprises about 1.5% N-Lauryl Sarcosine. In some embodiments, a buffer suitable for mRNA precipitation comprises about 2%, about 2.5% or about 5% N-Lauryl Sarcosine.

In some embodiments, a suitable solution for mRNA precipitation comprises a reducing agent. In some embodiments, the reducing agent is selected from dithiothreitol (DTT), beta-mercaptoethanol (b-ME), Tris(2-carboxyethyl)phosphine (TCEP), Tris(3-hydroxypropyl)phosphine (THPP), dithioerythritol (DTE) and dithiobutylamine (DTBA). In some embodiments, the reducing agent is dithiothreitol (DTT).

In some embodiments, DTT is present at a final concentration that is greater than 1 mM and up to about 200 mM. In some embodiments, DTT is present at a final concentration between 2.5 mM and 100 mM. In some embodiments, DTT is present at a final concentration between 5 mM and 50 mM. In some embodiments, DTT is present at a final concentration of about 20 mM.

Protein denaturation may occur even at a low concentration of the denaturation reagent, when in the presence or absence of the reducing agent. The combination of a high concentration of GSCN and a high concentration of DTT in a denaturing solution for precipitating an mRNA containing impurities yields mRNA which is pure and substantially free of protein contaminants. mRNA precipitated in the buffer can be processed through a filter. In some embodiments, the eluent after a single precipitation followed by filtration using the buffer comprising about 5 M GSCN and about 10 mM DTT is of high quality and purity with no detectable proteins impurities. Additionally, the method is reproducible at wide range of the amount of mRNA processed, in the scales involving about 1 gram, or about 10 grams, or about 100 grams, or about 500 grams, or about 1000 grams of mRNA and more, without causing hindrance in flow of fluids through a filter.

In some embodiments, the buffer for the precipitating step further comprises an alcohol. In some embodiments, the precipitating is performed under conditions where the mRNA, denaturing buffer (comprising GSCN and reducing agent, e.g. DTT) and alcohol (e.g., 100% ethanol) are present in a volumetric ratio of 1:(5):(3). In some embodiments, the precipitating is performed under conditions where the mRNA, denaturing buffer and alcohol (e.g., 100% ethanol) are present in a volumetric ratio of 1:(3.5):(2.1). In some embodiments, the precipitating is performed under conditions where the mRNA, denaturing buffer and alcohol (e.g., 100% ethanol) are present in a volumetric ratio of 1:(4):(2). In some embodiments, the precipitating is performed under conditions where the mRNA, denaturing buffer and alcohol (e.g., 100% ethanol) are present in a volumetric ratio of 1: (2.8):(1.9). In some embodiments, the precipitating is performed under conditions where the mRNA, denaturing buffer and alcohol (e.g., 100% ethanol) are present in the volumetric ratio of 1:(2.3):(1.7). In some embodiments, the precipitating is performed under conditions where the mRNA, denaturing buffer and alcohol (e.g., 100% ethanol) are present in the volumetric ratio of 1:(2.1):(1.5).

In some embodiments, it is desirable to incubate the impure preparation with one or more denaturing reagents described herein for a period of time at a desired temperature that permits precipitation of substantial amount of mRNA. For example, the mixture of an impure preparation and a denaturing agent may be incubated at room temperature or ambient temperature for a period of time. In some embodiments, a suitable incubation time is a period of or greater than about 2, 3, 4, 5, 6, 7, 8, 9, 10, 15, 20, 25, 30, 40, 50, or 60 minutes. In some embodiments, a suitable incubation time is a period of or less than about 60, 55, 50, 45, 40, 35, 30, 25, 20, 15, 10, 9, 8, 7, 6, or 5 minutes. In some embodiments, the mixture is incubated for about 5 minutes at room temperature. Typically, “room temperature” or “ambient temperature” refers to a temperature with the range of about 20-25° C., for example, about 20° C., 21° C., 22° C., 23° C., 24° C., or 25° C. In some embodiments, the mixture of an impure preparation and a denaturing agent may also be incubated above room temperature (e.g., about 30-37° C. or in particular, at about 30° C., 31° C., 32° C., 33° C., 34° C., 35° C., 36° C., or 37° C.) or below room temperature (e.g., about 15-20° C., or in particular, at about 15° C., 16° C., 17° C., 18° C., 19° C., or 20° C.). The incubation period may be adjusted based on the incubation temperature. Typically, a higher incubation temperature requires shorter incubation time.

Alternatively or additionally, a solvent may be used to facilitate mRNA precipitation. Suitable exemplary solvent includes, but is not limited to, isopropyl alcohol, acetone, methyl ethyl ketone, methyl isobutyl ketone, ethanol, methanol, denatonium, and combinations thereof. For example, a solvent (e.g., 100% ethanol) may be added to an impure preparation together with a denaturing reagent or after the addition of a denaturing reagent and the incubation as described herein, to further enhance and/or expedite mRNA precipitation. Typically, after the addition of a suitable solvent (e.g., 100% ethanol), the mixture may be incubated at room temperature for another period of time. Typically, a suitable period of incubation time is or greater than about 2, 3, 4, 5, 6, 7, 8, 9, 10, 15, 20, 25, 30, 40, 50, or 60 minutes. In some embodiments, a suitable period of incubation is a period of or less than about 60, 55, 50, 45, 40, 35, 30, 25, 20, 15, 10, 9, 8, 7, 6, or 5 minutes. Typically, the mixture is incubated at room temperature for about 5 minutes. Temperature above or below room may be used with proper adjustment of incubation time. Alternatively, incubation could occur at 4° C. or −20° C. for precipitation.

In some embodiments, the method of purifying mRNA is alcohol-free. Accordingly, in some embodiments, precipitating the mRNA in a suspension comprises one or more amphiphilic polymers in place of alcohol (e.g., 100% ethanol). Many amphiphilic polymers are known in the art. In some embodiments, amphiphilic polymer include pluronics, polyvinyl pyrrolidone, polyvinyl alcohol, polyethylene glycol (PEG), or combinations thereof. In some embodiments, the amphiphilic polymer is selected from one or more of the following: PEG triethylene glycol, tetraethylene glycol, PEG 200, PEG 300, PEG 400, PEG 600, PEG 1,000, PEG 1,500, PEG 2,000, PEG 3,000, PEG 3,350, PEG 4,000, PEG 6,000, PEG 8,000, PEG 10,000, PEG 20,000, PEG 35,000, and PEG 40,000, or combination thereof.

In some embodiments, the amphiphilic polymer comprises a mixture of two or more kinds of molecular weight PEG polymers are used. For example, in some embodiments, two, three, four, five, six, seven, eight, nine, ten, eleven, or twelve molecular weight PEG polymers comprise the amphiphilic polymer. Accordingly, in some embodiments, the PEG solution comprises a mixture of one or more PEG polymers. In some embodiments, the mixture of PEG polymers comprises polymers having distinct molecular weights. In some embodiments, the precipitating the mRNA in a suspension comprises a PEG polymer. Various kinds of PEG polymers are recognized in the art, some of which have distinct geometrical configurations. PEG polymers include, for example, PEG polymers having linear, branched, Y-shaped, or multi-arm configuration. In some embodiments, the PEG is in a suspension comprising one or more PEG of distinct geometrical configurations. In some embodiments, precipitating mRNA can be achieved using PEG-6000 to precipitate the mRNA. In some embodiments, precipitating mRNA can be achieved using PEG-400 to precipitate the mRNA.

In other embodiments, an alcohol-free method of purifying mRNA comprises precipitating mRNA with triethylene glycol (TEG). In some embodiments, precipitating mRNA can be achieved using triethylene glycol monomethyl ether (MTEG) to precipitate the mRNA. In some embodiments, precipitating mRNA can be achieved using tert-butyl-TEG-O-propionate to precipitate the mRNA. In some embodiments, precipitating mRNA can be achieved using TEG-dimethacrylate to precipitate the mRNA. In some embodiments, precipitating mRNA can be achieved using TEG-dimethyl ether to precipitate the mRNA. In some embodiments, precipitating mRNA can be achieved using TEG-divinyl ether to precipitate the mRNA. In some embodiments, precipitating mRNA can be achieved using TEG-monobutyl ether to precipitate the mRNA. In some embodiments, precipitating mRNA can be achieved using TEG-methyl ether methacrylate to precipitate the mRNA. In some embodiments, precipitating mRNA can be achieved using TEG-monodecyl ether to precipitate the mRNA. In some embodiments, precipitating mRNA can be achieved using TEG-dibenzoate to precipitate the mRNA. Any one of these PEG or TEG based reagents can be used in combination with GSCN to precipitate the mRNA. An exemplary ethanol-free method of purifying mRNA produced in accordance with the invention uses a combination of GSCN and MTEG to precipitate the mRNA.

In some embodiments, precipitating the mRNA in a suspension comprises a PEG polymer, wherein the PEG polymer comprises a PEG-modified lipid. In some embodiments, the PEG-modified lipid is 1,2-dimyristoyl-sn-glycerol, methoxypolyethylene glycol (DMG-PEG-2K). In some embodiments, the PEG modified lipid is a DOPA-PEG conjugate. In some embodiments, the PEG-modified lipid is a poloxamer-PEG conjugate. In some embodiments, the PEG-modified lipid comprises DOTAP. In some embodiments, the PEG-modified lipid comprises cholesterol.

In some embodiments, the mRNA is precipitated in a suspension comprising any of the aforementioned PEG or TEG reagents. In some embodiments, PEG or TEG is in the suspension at about 10% to about 100% weight/volume concentration. For example, in some embodiments, PEG or TEG is present in the suspension at about 5%, 10%, 15%, 20%, 25%, 30%, 35%, 40%, 45%, 50%, 55%, 60%, 65%, 70%, 75%, 80%, 85%, 90%, 95% 100% weight/volume concentration, and any values there between.

In some embodiments, precipitating the mRNA in a suspension comprises a volume:volume ratio of PEG or TEG to total mRNA suspension volume of about 0.1 to about 5.0. For example, in some embodiments, PEG or TEG is present in the mRNA suspension at a volume: volume ratio of about 0.1, 0.2, 0.3, 0.4, 0.5, 0.6, 0.7, 0.8, 0.9, 1.0, 1.25, 1.5, 1.75, 2.0, 2.25, 2.5, 2.75, 3.0, 3.25, 3.5, 3.75, 4.0, 4.25, 4.5, 4.75, 5.0.

In some embodiments, a reaction volume for mRNA precipitation comprises (i) GSCN and (ii) PEG or TEG.

Characterization of mRNA

Full-length, abortive and/or prematurely terminated transcripts of mRNA may be detected and quantified using any methods available in the art. In some embodiments, the synthesized mRNA molecules are detected using blotting, capillary electrophoresis, chromatography, fluorescence, gel electrophoresis, HPLC, silver stain, spectroscopy, ultraviolet (UV), or UPLC, or a combination thereof. Other detection methods known in the art are included in the present invention. In some embodiments, the synthesized mRNA molecules are detected using UV absorption spectroscopy with separation by capillary electrophoresis. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

In some embodiments, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than full-length mRNA. The impurities include IVT contaminants, e.g., proteins, enzymes, free nucleotides and/or shortmers.

In some embodiments, mRNA produced according to the invention is substantially free of shortmers or abortive transcripts. In particular, mRNA produced according to the invention contains undetectable level of shortmers or abortive transcripts by capillary electrophoresis or Glyoxal gel electrophoresis. As used herein, the term “shortmers” or “abortive transcripts” refers to any transcripts that are less than full-length. In some embodiments, “shortmers” or “abortive transcripts” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail.

Elongated mRNA Transcripts

In some embodiments, at least 75%, at least 80%, at least 85%, at least 90%, at least 95% of the mRNA transcripts generated by the methods disclosed herein are terminated at the termination signal. As used herein “terminated at the termination signal” refers to termination of transcription within 10 nucleotides, 9 nucleotides, 8 nucleotides, 7 nucleotides, 6 nucleotides, 5 nucleotides, 4 nucleotides, 3 nucleotides, 2 nucleotides, 1 nucleotides or 0 nucleotides of the 3′ end of the termination signal.

In order to detect runoff transcription, the mRNA transcripts can be digested to produce short 3′ end fragments, which are analyzed using liquid chromatography mass spectrometry (“digestion LC/MS”). Suitable 3′ end fragments have a size of less than 100 nucleotides, e.g., less than 90 nucleotides, 80 nucleotides, 70 nucleotides, 60 nucleotides, 50 nucleotides, 40 nucleotides. 3′ end fragments of the desired length can be produced by providing a probe oligonucleotide which specifically hybridizes to the 3′ end of the templated mRNA transcript such that a DNA/RNA hybrid is formed. The probe oligonucleotide may bind within from about 5 to about 20 nucleotides of the 3′ end of the templated mRNA transcript. The DNA/RNA hybrid can then be digested with RNaseH to yield a 3′ end fragment of the desired length. The 3′ end fragments can be analyzed using RNA sequencing to determine the presence and length of the runoff sequence. A suitable method for RNA sequencing is nanopore sequencing.

Suitable probe oligo nucleotides are modified RNA-DNA gap oligonucleotides (also commonly referred to as gapmers). A typical gapmer design consists of a 5′-wing followed by a gap of 8 to 12 deoxynucleic acid monomers that may be natural nucleic acids or contain a sulphur ion in the phosphor group (PS linkage) followed by a 3′-wing. Such an RNA-DNA-RNA-like configuration typically contain RNA nucleotides which are modified, e.g. by containing 2′-O-methyl ribose. The RNA-DNA gap oligonucleotide disclosed herein defer from this standard design as they have a shorter gap of only 3-5 deoxynucleic acid monomers (typically 4 deoxynucleic acid monomers). This enables precise targeting of RNAse H digestion to the 3′ end of the templated mRNA transcript. To ensure precise annealing to the mRNA transcript, the RNA-DNA gap oligonucleotide is 10-20 nucleotides long, e.g., about 15-18 nucleotides. The 5′ and 3′ wing sequences comprising modified RNA nucleotides do not have the same length. In some embodiments, the 5′ wing is shorter (e.g., has a length of 4-6 nucleotides) than the 3′ wing (which, e.g., has a length of 7-10 nucleotides). In other embodiments, the 5′ wing is longer (e.g., has a length of 7-10 nucleotides) than the 3′ wing (which, e.g., has a length of 4-6 nucleotides).

### Protein Expression

mRNA transcripts synthesized with T7 RNA polymerases are typically contaminated with RNAs longer and shorter than the desired transcript (see WO 2018/157153). Elongated sequences are thought to be generated by non-templated additions of nucleotides at the end of the template-encoded mRNA transcript after a termination signal. The additional nucleotides are commonly referred to as “runoff”. Further extension can occur when the 3′ end of the runoff has sufficient complementarity to bind to itself or a second mRNA molecule to form extendible intra- or intermolecular duplexes, respectively (Gholamalipour et al. 2018, Nucleic Acids Research 46:18, pp 9253-9263). When double-stranded RNA (dsRNA) enters the cell, it is sensed as a viral invader. This leads to the activation of dsRNA-dependent enzymes, such as oligoadenylate synthetase (OAS), RNA-specific adenosine deaminase (ADAR), and RNA-activated protein kinase (PKR), which results in the inhibition of protein synthesis (Baiersdorfer et al. 2019, Molecular Therapy: Nucleic Acids, 15: 26-35).

The examples of the present application demonstrate that synthesis of mRNA according to the present invention prevents the undesired elongation of mRNA transcripts from both linear and super-coiled DNA templates. Without undesired elongation of its 3′ ends, mRNA synthesized according to the present invention, including mRNA synthesized using T7 RNA polymerase, is essentially free of dsRNA, as can be determined with a monoclonal antibody specific for dsRNA (e.g., by using a dot blot assay). Accordingly, it does not activate dsRNA-dependent enzymes when administered to a subject. mRNA synthesized according to the present invention therefore results in more efficient protein translation.

In some embodiments, mRNA synthesized according to the present invention results in an increased protein expression once transfected into cells, e.g., by at least 2-fold, 3-fold, 4-fold, 5-fold, 10-fold, 20-fold, 30-fold, 40-fold, 50-fold, 100-fold, 500-fold, 1000-fold, or more, relative to the same amount of mRNA synthesized using a prior art process, in particular those employing T7 or T3 RNA Polymerase.

In some embodiments, mRNA synthesized according to the present invention results in an increased protein activity encoded by the mRNA once transfected into cells, e.g., by at least 2-fold, 3-fold, 4-fold, 5-fold, 10-fold, 20-fold, 30-fold, 40-fold, 50-fold, 100-fold, 500-fold, 1000-fold, or more, relative to the same amount of mRNA synthesized using a prior art process, in particular those employing T7 or T3 RNA Polymerase.

Any mRNA may be synthesized using the present invention. In some embodiments, an mRNA encodes one or more naturally occurring peptides. In some embodiments, an mRNA encodes one or more modified or non-natural peptides.

In some embodiments an mRNA encodes an intracellular protein. In some embodiments, an mRNA encodes a cytosolic protein. In some embodiments, an mRNA encodes a protein associated with the actin cytoskeleton. In some embodiments, an mRNA encodes a protein associated with the plasma membrane. In some specific embodiments, an mRNA encodes a transmembrane protein. In some specific embodiments an mRNA encodes an ion channel protein. In some embodiments, an mRNA encodes a perinuclear protein. In some embodiments, an mRNA encodes a nuclear protein. In some specific embodiments, an mRNA encodes a transcription factor. In some embodiments, an mRNA encodes a chaperone protein. In some embodiments, an mRNA encodes an intracellular enzyme (e.g., mRNA encoding an enzyme associated with urea cycle or lysosomal storage metabolic disorders). In some embodiments, an mRNA encodes a protein involved in cellular metabolism, DNA repair, transcription and/or translation. In some embodiments, an mRNA encodes an extracellular protein. In some embodiments, an mRNA encodes a protein associated with the extracellular matrix. In some embodiments an mRNA encodes a secreted protein. In specific embodiments, an mRNA used in the composition and methods of the invention may be used to express functional proteins or enzymes that are excreted or secreted by one or more target cells into the surrounding extracellular fluid (e.g., mRNA encoding hormones and/or neurotransmitters).

The present invention provides methods for producing a therapeutic composition enriched with full-length mRNA molecules encoding a peptide or polypeptide of interest for use in the delivery to or treatment of a subject, e.g., a human subject or a cell of a human subject or a cell that is treated and delivered to a human subject.

Accordingly, in certain embodiments the present invention provides a method for producing a therapeutic composition enriched with full-length mRNA that encodes a peptide or polypeptide for use in the delivery to or treatment of the lung of a subject or a lung cell. In certain embodiments the present invention provides a method for producing a therapeutic composition enriched with full-length mRNA that encodes for cystic fibrosis transmembrane conductance regulator (CFTR) protein. In certain embodiments the present invention provides a method for producing a therapeutic composition enriched with full-length mRNA that encodes for ATP-binding cassette sub-family A member 3 protein. In certain embodiments the present invention provides a method for producing a therapeutic composition enriched with full-length mRNA that encodes for dynein axonemal intermediate chain 1 protein. In certain embodiments the present invention provides a method for producing a therapeutic composition enriched with full-length mRNA that encodes for dynein axonemal heavy chain 5 (DNAH5) protein. In certain embodiments the present invention provides a method for producing a therapeutic composition enriched with full-length mRNA that encodes for alpha-1-antitrypsin protein. In certain embodiments the present invention provides a method for producing a therapeutic composition enriched with full-length mRNA that encodes for forkhead box P3 (FOXP3) protein. In certain embodiments the present invention provides a method for producing a therapeutic composition enriched with full-length mRNA that encodes one or more surfactant protein, e.g., one or more of surfactant A protein, surfactant B protein, surfactant C protein, and surfactant D protein.

In certain embodiments the present invention provides a method for producing a therapeutic composition enriched with full-length mRNA that encodes a peptide or polypeptide for use in the delivery to or treatment of the liver of a subject or a liver cell. Such peptides and polypeptides can include those associated with a urea cycle disorder, associated with a lysosomal storage disorder, with a glycogen storage disorder, associated with an amino acid metabolism disorder, associated with a lipid metabolism or fibrotic disorder, associated with methylmalonic acidemia, or associated with any other metabolic disorder for which delivery to or treatment of the liver or a liver cell with enriched full-length mRNA provides therapeutic benefit.

In certain embodiments the present invention provides a method for producing a therapeutic composition enriched with full-length mRNA that encodes for a protein associated with a urea cycle disorder. In certain embodiments the present invention provides a method for producing a therapeutic composition enriched with full-length mRNA that encodes for ornithine transcarbamylase (OTC) protein. In certain embodiments the present invention provides a method for producing a therapeutic composition enriched with full-length mRNA that encodes for arginosuccinate synthetase 1 protein. In certain embodiments the present invention provides a method for producing a therapeutic composition enriched with full-length mRNA that encodes for carbamoyl phosphate synthetase I protein. In certain embodiments the present invention provides a method for producing a therapeutic composition enriched with full-length mRNA that encodes for arginosuccinate lyase protein. In certain embodiments the present invention provides a method for producing a therapeutic composition enriched with full-length mRNA that encodes for arginase protein.

In certain embodiments the present invention provides a method for producing a therapeutic composition enriched with full-length mRNA that encodes for a protein associated with a lysosomal storage disorder. In certain embodiments the present invention provides a method for producing a therapeutic composition enriched with full-length mRNA that encodes for alpha galactosidase protein. In certain embodiments the present invention provides a method for producing a therapeutic composition enriched with full-length mRNA that encodes for glucocerebrosidase protein. In certain embodiments the present invention provides a method for producing a therapeutic composition enriched with full-length mRNA that encodes for iduronate-2-sulfatase protein. In certain embodiments the present invention provides a method for producing a therapeutic composition enriched with full-length mRNA that encodes for iduronidase protein. In certain embodiments the present invention provides a method for producing a therapeutic composition enriched with full-length mRNA that encodes for N-acetyl-alpha-D-glucosaminidase protein. In certain embodiments the present invention provides a method for producing a therapeutic composition enriched with full-length mRNA that encodes for heparan N-sulfatase protein. In certain embodiments the present invention provides a method for producing a therapeutic composition enriched with full-length mRNA that encodes for galactosamine-6 sulfatase protein. In certain embodiments the present invention provides a method for producing a therapeutic composition enriched with full-length mRNA that encodes for beta-galactosidase protein. In certain embodiments the present invention provides a method for producing a therapeutic composition enriched with full-length mRNA that encodes for lysosomal lipase protein. In certain embodiments the present invention provides a method for producing a therapeutic composition enriched with full-length mRNA that encodes for arylsulfatase B (N-acetylgalactosamine-4-sulfatase) protein. In certain embodiments the present invention provides a method for producing a therapeutic composition enriched with full-length mRNA that encodes for transcription factor EB (TFEB).

In certain embodiments the present invention provides a method for producing a therapeutic composition enriched with full-length mRNA that encodes for a protein associated with a glycogen storage disorder. In certain embodiments the present invention provides a method for producing a therapeutic composition enriched with full-length mRNA that encodes for acid alpha-glucosidase protein. In certain embodiments the present invention provides a method for producing a therapeutic composition enriched with full-length mRNA that encodes for glucose-6-phosphatase (G6PC) protein. In certain embodiments the present invention provides a method for producing a therapeutic composition enriched with full-length mRNA that encodes for liver glycogen phosphorylase protein. In certain embodiments the present invention provides a method for producing a therapeutic composition enriched with full-length mRNA that encodes for muscle phosphoglycerate mutase protein. In certain embodiments the present invention provides a method for producing a therapeutic composition enriched with full-length mRNA that encodes for glycogen debranching enzyme.

In certain embodiments the present invention provides a method for producing a therapeutic composition enriched with full-length mRNA that encodes for a protein associated with amino acid metabolism. In certain embodiments the present invention provides a method for producing a therapeutic composition enriched with full-length mRNA that encodes for phenylalanine hydroxylase enzyme. In certain embodiments the present invention provides a method for producing a therapeutic composition enriched with full-length mRNA that encodes for glutaryl-CoA dehydrogenase enzyme. In certain embodiments the present invention provides a method for producing a therapeutic composition enriched with full-length mRNA that encodes for propionyl-CoA caboxylase enzyme. In certain embodiments the present invention provides a method for producing a therapeutic composition enriched with full-length mRNA that encodes for oxalase alanine-glyoxylate aminotransferase enzyme.

In certain embodiments the present invention provides a method for producing a therapeutic composition enriched with full-length mRNA that encodes for a protein associated with a lipid metabolism or fibrotic disorder. In certain embodiments the present invention provides a method for producing a therapeutic composition enriched with full-length mRNA that encodes for a mTOR inhibitor. In certain embodiments the present invention provides a method for producing a therapeutic composition enriched with full-length mRNA that encodes for ATPase phospholipid transporting 8B1 (ATP8B1) protein. In certain embodiments the present invention provides a method for producing a therapeutic composition enriched with full-length mRNA that encodes for one or more NF-kappa B inhibitors, such as one or more of I-kappa B alpha, interferon-related development regulator 1 (IFRD1), and Sirtuin 1 (SIRT1). In certain embodiments the present invention provides a method for producing a therapeutic composition enriched with full-length mRNA that encodes for PPAR-gamma protein or an active variant.

In certain embodiments the present invention provides a method for producing a therapeutic composition enriched with full-length mRNA that encodes for a protein associated with methylmalonic acidemia. For example, in certain embodiments the present invention provides a method for producing a therapeutic composition enriched with full-length mRNA that encodes for methylmalonyl CoA mutase protein. In certain embodiments the present invention provides a method for producing a therapeutic composition enriched with full-length mRNA that encodes for methylmalonyl CoA epimerase protein.

In certain embodiments the present invention provides a method for producing a therapeutic composition enriched with full-length mRNA for which delivery to or treatment of the liver can provide therapeutic benefit. In certain embodiments the present invention provides a method for producing a therapeutic composition enriched with full-length mRNA that encodes for ATP7B protein, also known as Wilson disease protein. In certain embodiments the present invention provides a method for producing a therapeutic composition enriched with full-length mRNA that encodes for porphobilinogen deaminase enzyme. In certain embodiments the present invention provides a method for producing a therapeutic composition enriched with full-length mRNA that encodes for one or clotting enzymes, such as Factor VIII, Factor IX, Factor VII, and Factor X. In certain embodiments the present invention provides a method for producing a therapeutic composition enriched with full-length mRNA that encodes for human hemochromatosis (HFE) protein.

In certain embodiments the present invention provides a method for producing a therapeutic composition enriched with full-length mRNA that encodes a peptide or polypeptide for use in the delivery to or treatment of the cardiovasculature of a subject or a cardiovascular cell. In certain embodiments the present invention provides a method for producing a therapeutic composition enriched with full-length mRNA that encodes for vascular endothelial growth factor A protein. In certain embodiments the present invention provides a method for producing a therapeutic composition enriched with full-length mRNA that encodes for relaxin protein. In certain embodiments the present invention provides a method for producing a therapeutic composition enriched with full-length mRNA that encodes for bone morphogenetic protein-9 protein. In certain embodiments the present invention provides a method for producing a therapeutic composition enriched with full-length mRNA that encodes for bone morphogenetic protein-2 receptor protein.

In certain embodiments the present invention provides a method for producing a therapeutic composition enriched with full-length mRNA that encodes a peptide or polypeptide for use in the delivery to or treatment of the muscle of a subject or a muscle cell. In certain embodiments the present invention provides a method for producing a therapeutic composition enriched with full-length mRNA that encodes for dystrophin protein. In certain embodiments the present invention provides a method for producing a therapeutic composition enriched with full-length mRNA that encodes for frataxin protein. In certain embodiments the present invention provides a method for producing a therapeutic composition enriched with full-length mRNA that encodes a peptide or polypeptide for use in the delivery to or treatment of the cardiac muscle of a subject or a cardiac muscle cell. In certain embodiments the present invention provides a method for producing a therapeutic composition enriched with full-length mRNA that encodes for a protein that modulates one or both of a potassium channel and a sodium channel in muscle tissue or in a muscle cell. In certain embodiments the present invention provides a method for producing a therapeutic composition enriched with full-length mRNA that encodes for a protein that modulates a Kv7.1 channel in muscle tissue or in a muscle cell. In certain embodiments the present invention provides a method for producing a therapeutic composition enriched with full-length mRNA that encodes for a protein that modulates a Nav 1.5 channel in muscle tissue or in a muscle cell.

In certain embodiments the present invention provides a method for producing a therapeutic composition enriched with full-length mRNA that encodes a peptide or polypeptide for use in the delivery to or treatment of the nervous system of a subject or a nervous system cell. For example, in certain embodiments the present invention provides a method for producing a therapeutic composition enriched with full-length mRNA that encodes for survival motor neuron 1 protein. For example, in certain embodiments the present invention provides a method for producing a therapeutic composition enriched with full-length mRNA that encodes for survival motor neuron 2 protein. In certain embodiments the present invention provides a method for producing a therapeutic composition enriched with full-length mRNA that encodes for frataxin protein. In certain embodiments the present invention provides a method for producing a therapeutic composition enriched with full-length mRNA that encodes for ATP binding cassette subfamily D member 1 (ABCD1) protein. In certain embodiments the present invention provides a method for producing a therapeutic composition enriched with full-length mRNA that encodes for CLN3 protein.

In certain embodiments the present invention provides a method for producing a therapeutic composition enriched with full-length mRNA that encodes a peptide or polypeptide for use in the delivery to or treatment of the blood or bone marrow of a subject or a blood or bone marrow cell. In certain embodiments the present invention provides a method for producing a therapeutic composition enriched with full-length mRNA that encodes for beta globin protein. In certain embodiments the present invention provides a method for producing a therapeutic composition enriched with full-length mRNA that encodes for Bruton's tyrosine kinase protein. In certain embodiments the present invention provides a method for producing a therapeutic composition enriched with full-length mRNA that encodes for one or clotting enzymes, such as Factor VIII, Factor IX, Factor VII, and Factor X.

In certain embodiments the present invention provides a method for producing a therapeutic composition enriched with full-length mRNA that encodes a peptide or polypeptide for use in the delivery to or treatment of the kidney of a subject or a kidney cell. In certain embodiments the present invention provides a method for producing a therapeutic composition enriched with full-length mRNA that encodes for collagen type IV alpha 5 chain (COL4A5) protein.

In certain embodiments the present invention provides a method for producing a therapeutic composition enriched with full-length mRNA that encodes a peptide or polypeptide for use in the delivery to or treatment of the eye of a subject or an eye cell. In certain embodiments the present invention provides a method for producing a therapeutic composition enriched with full-length mRNA that encodes for ATP-binding cassette sub-family A member 4 (ABCA4) protein. In certain embodiments the present invention provides a method for producing a therapeutic composition enriched with full-length mRNA that encodes for retinoschisin protein. In certain embodiments the present invention provides a method for producing a therapeutic composition enriched with full-length mRNA that encodes for retinal pigment epithelium-specific 65 kDa (RPE65) protein. In certain embodiments the present invention provides a method for producing a therapeutic composition enriched with full-length mRNA that encodes for centrosomal protein of 290 kDa (CEP290).

In certain embodiments the present invention provides a method for producing a therapeutic composition enriched with full-length mRNA that encodes a peptide or polypeptide for use in the delivery of or treatment with a vaccine for a subject or a cell of a subject. For example, in certain embodiments the present invention provides a method for producing a therapeutic composition enriched with full-length mRNA that encodes for an antigen from an infectious agent, such as a virus. In certain embodiments the present invention provides a method for producing a therapeutic composition enriched with full-length mRNA that encodes for an antigen from influenza virus. In certain embodiments the present invention provides a method for producing a therapeutic composition enriched with full-length mRNA that encodes for an antigen from respiratory syncytial virus. In certain embodiments the present invention provides a method for producing a therapeutic composition enriched with full-length mRNA that encodes for an antigen from rabies virus. In certain embodiments the present invention provides a method for producing a therapeutic composition enriched with full-length mRNA that encodes for an antigen from cytomegalovirus. In certain embodiments the present invention provides a method for producing a therapeutic composition enriched with full-length mRNA that encodes for an antigen from rotavirus. In certain embodiments the present invention provides a method for producing a therapeutic composition enriched with full-length mRNA that encodes for an antigen from a hepatitis virus, such as hepatitis A virus, hepatitis B virus, or hepatis C virus. In certain embodiments the present invention provides a method for producing a therapeutic composition enriched with full-length mRNA that encodes for an antigen from human papillomavirus. In certain embodiments the present invention provides a method for producing a therapeutic composition enriched with full-length mRNA that encodes for an antigen from a herpes simplex virus, such as herpes simplex virus 1 or herpes simplex virus 2. In certain embodiments the present invention provides a method for producing a therapeutic composition enriched with full-length mRNA that encodes for an antigen from a human immunodeficiency virus, such as human immunodeficiency virus type 1 or human immunodeficiency virus type 2. In certain embodiments the present invention provides a method for producing a therapeutic composition enriched with full-length mRNA that encodes for an antigen from a human metapneumovirus. In certain embodiments the present invention provides a method for producing a therapeutic composition enriched with full-length mRNA that encodes for an antigen from a human parainfluenza virus, such as human parainfluenza virus type 1, human parainfluenza virus type 2, or human parainfluenza virus type 3. In certain embodiments the present invention provides a method for producing a therapeutic composition enriched with full-length mRNA that encodes for an antigen from malaria virus. In certain embodiments the present invention provides a method for producing a therapeutic composition enriched with full-length mRNA that encodes for an antigen from zika virus. In certain embodiments the present invention provides a method for producing a therapeutic composition enriched with full-length mRNA that encodes for an antigen from chikungunya virus.

In certain embodiments the present invention provides a method for producing a therapeutic composition enriched with full-length mRNA that encodes for an antigen associated with a cancer of a subject or identified from a cancer cell of a subject. In certain embodiments the present invention provides a method for producing a therapeutic composition enriched with full-length mRNA that encodes for an antigen determined from a subject's own cancer cell, i.e., to provide a personalized cancer vaccine. In certain embodiments the present invention provides a method for producing a therapeutic composition enriched with full-length mRNA that encodes for an antigen expressed from a mutant KRAS gene.

In certain embodiments the present invention provides a method for producing a therapeutic composition enriched with full-length mRNA that encodes for an antibody. In certain embodiments, the antibody can be a bi-specific antibody. In certain embodiments, the antibody can be part of a fusion protein. In certain embodiments the present invention provides a method for producing a therapeutic composition enriched with full-length mRNA that encodes for an antibody to OX40. In certain embodiments the present invention provides a method for producing a therapeutic composition enriched with full-length mRNA that encodes for an antibody to VEGF. In certain embodiments the present invention provides a method for producing a therapeutic composition enriched with full-length mRNA that encodes for an antibody to tissue necrosis factor alpha. In certain embodiments the present invention provides a method for producing a therapeutic composition enriched with full-length mRNA that encodes for an antibody to CD3. In certain embodiments the present invention provides a method for producing a therapeutic composition enriched with full-length mRNA that encodes for an antibody to CD 19.

In certain embodiments the present invention provides a method for producing a therapeutic composition enriched with full-length mRNA that encodes for an immunomodulator. In certain embodiments the present invention provides a method for producing a therapeutic composition enriched with full-length mRNA that encodes for Interleukin 12. In certain embodiments the present invention provides a method for producing a therapeutic composition enriched with full-length mRNA that encodes for Interleukin 23. In certain embodiments the present invention provides a method for producing a therapeutic composition enriched with full-length mRNA that encodes for Interleukin 36 gamma. In certain embodiments the present invention provides a method for producing a therapeutic composition enriched with full-length mRNA that encodes for a constitutively active variant of one or more stimulator of interferon genes (STING) proteins.

In certain embodiments the present invention provides a method for producing a therapeutic composition enriched with full-length mRNA that encodes for an endonuclease. In certain embodiments the present invention provides a method for producing a therapeutic composition enriched with full-length mRNA that encodes for an RNA-guided DNA endonuclease protein, such as Cas 9 protein. In certain embodiments the present invention provides a method for producing a therapeutic composition enriched with full-length mRNA that encodes for a meganuclease protein. In certain embodiments the present invention provides a method for producing a therapeutic composition enriched with full-length mRNA that encodes for a transcription activator-like effector nuclease protein. In certain embodiments the present invention provides a method for producing a therapeutic composition enriched with full-length mRNA that encodes for a zinc finger nuclease protein.

### Lipid Nanoparticles

mRNA synthesized according to the present invention may be formulated and delivered for in vivo protein production using any method. In some embodiments, mRNA is encapsulated, into a transfer vehicle, such as a nanoparticle. Among other things, one purpose of such encapsulation is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue. In some embodiments, nanoparticles may be lipid-based nanoparticles, e.g., comprising a liposome, or polymer-based nanoparticles. In some embodiments, a nanoparticle may have a diameter of less than about 40-100 nm. A nanoparticle may include at least 1 μg, 10 μg, 100 μg, 1 mg, 10 mg, 100 mg, 1 g, or more mRNA.

In some embodiments, the transfer vehicle is a liposomal vesicle, or other means to facilitate the transfer of a nucleic acid to target cells and tissues. Suitable transfer vehicles include, but are not limited to, liposomes, nanoliposomes, ceramide-containing nanoliposomes, proteoliposomes, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), nanodendrimers, starch-based delivery systems, micelles, emulsions, niosomes, plasmids, viruses, calcium phosphate nucleotides, aptamers, peptides and other vectorial tags. Also contemplated is the use of bionanocapsules and other viral capsid proteins assemblies as a suitable transfer vehicle. (Hum. Gene Ther. 2008 September; 19(9):887-95).

A liposome may include one or more cationic lipids, one or more non-cationic lipids, one or more sterol-based lipids, and/or one or more PEG-modified lipids. A liposome may include three or more distinct components of lipids, one distinct component of lipids being sterol-based cationic lipids. In some embodiments, the sterol-based cationic lipid is an imidazole cholesterol ester or “ICE” lipid (see, WO 2011/068810, which is incorporated by reference in its entirety). In some embodiments, sterol-based cationic lipids constitute no more than 70% (e.g., no more than 65% and 60%) of the total lipids in a lipid nanoparticle (e.g., liposome).

Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

Non-limiting examples of cationic lipids include C12-200, MC3, DLinDMA, DLinkC2DMA, cKK-E12, ICE (Imidazole-based), HGT5000, HGT5001, OF-02, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, and HGT4003, or a combination thereof.

Non-limiting examples of non-cationic lipids include ceramide; cephalin; cerebrosides; diacylglycerols; 1,2-dipalmitoyl-sn-glycero-3-phosphorylglycerol sodium salt (DPPG); 1,2-distearoyl-sn-glycero-3-phosphoethanolamine (DSPE); 1,2-distearoyl-sn-glycerol-3-phosphocholine (DSPC); 1,2-dipalmitoyl-sn-glycero-3-phosphocholine (DPPC); 1,2-dioleyl-sn-glycero-3-phosphoethanolamine (DOPE); 1,2-Dierucoyl-sn-glycero-3-phosphoethanolamine (DEPE), 1,2-dioleyl-sn-glycero-3-phosphotidylcholine (DOPC); 1,2-dipalmitoyl-sn-glycero-3-phosphoethanolamine (DPPE); 1,2-dimyristoyl-sn-glycero-3-phosphoethanolamine (DMPE); and 1,2-dioleoyl-5n-glycero-3-phospho-(1′-rac-glycerol) (DOPG), 1-palmitoyl-2-oleoyl-phosphatidylethanolamine (POPE); 1-palmitoyl-2-oleoyl-sn-glycero-3-phosphocholine (POPC); 1-stearoyl-2-oleoyl-phosphatidylethanolamine (SOPE); sphingomyelin; or a combination thereof.

In some embodiments, a PEG-modified lipid may be a poly(ethylene) glycol chain of up to 5 kDa in length covalently attached to a lipid with alkyl chain(s) of C₆-C₂₀ length. Non-limiting examples of PEG-modified lipids include DMG-PEG, DMG-PEG2K, C8-PEG, DOG PEG, ceramide PEG, and DSPE-PEG, or a combination thereof.

Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins and polyethylenimine. A polymer-based nanoparticles may include polyethylenimine (PEI), e.g., a branched PEI.

Typically, the lipid portion of a liposomes in accordance with the present invention consists of either 3 or 4 lipid components. A 4-component liposome in accordance with the invention generally has the following lipid components: a cationic lipid (typically an ionizable cationic lipid such as cKK-E12 or cyclic amino acid-based lipids), a non-cationic lipid (e.g., DOPE or DEPE), a cholesterol-based lipid (e.g., cholesterol) and a PEG-modified lipid (e.g., DMG-PEG2K). A 3-component liposome in accordance with the invention generally has the following lipid components: a sterol-based lipid (e.g., ICE or other imidazole-based cholesterol derivative), a non-cationic lipid (e.g., DOPE or DEPE) and a PEG-modified lipid (e.g., DMG-PEG2K).

Additional teaching relevant to the present invention are described in one or more of the following: WO 2011/068810, WO 2012/075040, U.S. Ser. No. 15/294,249, U.S. 62/421,021 and U.S. Ser. No. 15/809,680, and the related applications filed Feb. 27, 2017 by Applicants entitled “METHODS FOR PURIFICATION OF MESSENGER RNA”, “NOVEL CODON-OPTIMIZED CFTR SEQUENCE”, and “METHODS FOR PURIFICATION OF MESSENGER RNA”, each of which is incorporated by reference in its entirety.

The liposomal transfer vehicles for use in the compositions of the invention can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MHLVs. Unilamellar vesicles (ULV) can then be formed by homogenization, sonication or extrusion of the multilamellar vesicles. In addition, unilamellar vesicles can be formed by detergent removal techniques.

Various methods are described in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference. As used herein, Process A refers to a conventional method of encapsulating mRNA by mixing mRNA with a mixture of lipids, without first pre-forming the lipids into lipid nanoparticles, as described in US 2016/0038432. As used herein, Process B refers to a process of encapsulating messenger RNA (mRNA) by mixing pre-formed lipid nanoparticles with mRNA, as described in US 2018/0153822.

The process of incorporation of a desired mRNA into a liposome is often referred to as “loading”. Exemplary methods are described in Lasic, et al. FEBS Lett., 312: 255-258, 1992, which is incorporated herein by reference. The liposome-incorporated mRNA may be completely or partially located in the interior space of the liposome, within the bilayer membrane of the liposome, or associated with the exterior surface of the liposome membrane. The incorporation of mRNA into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate its delivery to the target cell or tissue.

### Pharmaceutical Compositions

By combining the various processes described herein to provide an optimized DNA sequence that is transcribed faithfully as templated by a process described herein, mRNA transcripts of superior quality are provided that are essentially free of dsRNA as well as contaminating shortmer and longmer sequences. Efficient recovery of such mRNA transcripts using the purification processes described herein (in particular the precipitation-based, ethanol-free method for purifying mRNA described here) results in highly pure mRNA transcripts with the same superior properties. Encapsulating these mRNA transcripts by mixing pre-formed lipid nanoparticles with the purified mRNA (e.g., by using Process B as described above) can results in exceptionally high encapsulation efficiencies (e.g., greater than 90%). The end result of combining these various processing steps is a pharmaceutical product that is extremely efficient in delivering mRNA to target cells to achieve maximum expression of the mRNA-encoded peptide, polypeptide or protein.

Accordingly, in some embodiments, the invention provides a method for preparing a pharmaceutical composition comprising the following steps:
a) providing a DNA sequence that comprises a protein coding sequence;
b) optimizing the DNA sequence by:
   i) determining the presence of a termination signal in the DNA sequence, wherein the termination signal has the following nucleic acid sequence: 5′-X₁ATCTX₂TX₃-3′ (SEQ ID NO: 1), wherein X₁, X₂ and X₃ are independently selected from A, C, T or G, and if one or more termination signal is present, modifying the DNA sequence by replacing one or more nucleic acids at any one of position 2, 3, 4, 5 and 7 of said termination signal(s) with any one of the other three nucleic acids to generate the optimized DNA sequence, wherein, if required, the one or more replacement nucleic acids are selected to preserve the amino acid sequence of the protein encoded by the protein coding sequence, and/or
   ii) adding one or more termination signals at the 3′ end of the protein coding sequence, wherein the one or more termination signal(s) comprises the following nucleic acid sequence: X₁ATCTX₂TX₃-3′ (SEQ ID NO: 1), wherein X₁, X₂ and X3 are independently selected from A, C, T or G;
c) synthesizing mRNA by in vitro transcription from the optimized DNA template of step (b);
d) precipitating mRNA from the preparation in step (c);
e) purifying the impure preparation comprising the precipitated mRNA of step (d) by tangential flow filtration;
f) encapsulating the purified mRNA of step (e) in a liposome comprising one or more cationic lipids, one or more non-cationic lipids, one or more sterol-based lipids, and one or more PEG-modified lipids.

In some embodiments, the method comprises a separate capping and tailing reaction performed after step (e). In these embodiments, steps (d) and (e) are repeated after the capping and tailing reaction. In some embodiments, the purifying the impure preparation involves an ethanol-free method. In some embodiments, encapsulating involves mixing the purified mRNA with pre-formed lipid nanoparticles. In some embodiment, step (f) is followed by a formulation step. The formulation step may involve a buffer exchange. In some embodiments, the formulation steps involves lyophilisation of the liposomes encapsulating the mRNA.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In addition, the materials, methods, and examples are illustrative only and are not intended to be limiting.

### EXAMPLES

### Example 1: Exemplary Experimental Design for mRNA Synthesis Using T7 and SP6 RNA Polymerase

This example illustrates exemplary conditions for T7- and SP6 RNA polymerase-based mRNA synthesis, transfection, and characterization of the same.

### Messenger RNA Material

A plasmid with a DNA sequence encoding a protein coding sequence of interest operably linked to RNA polymerase promoter was linearized with a restriction enzyme and purified. mRNA transcripts were synthesized by in vitro transcription from the purified and linearized plasmid. The T7 transcription reaction consisted of 1× T7 transcription buffer (80 mM HEPES pH 8.0, 2 mM Spermidine, and 25 mM MgCh with a final pH of 7.7), 10 mM DTT, 7.25 mM each ATP, GTP, CTP, and UTP, RNAse Inhibitor, Pyrophosphatase, and T7 RNA Polymerase. The SP6 reaction included 5 mM of each NTP, about 0.05 mg/mL SP6 RNA polymerase DNA, and about 0.1 mg/mL template DNA; other components of transcription buffer varied. The reactions were performed for 60 to 90 minutes (unless otherwise noted) at 37° C. DNAsel was added to stop the reaction and incubated for 15 more minutes at 37° C. The in vitro transcribed mRNA was purified using the Qiagen RNA maxi column following manufacturer's recommendations.

The purified mRNA transcripts from the aforementioned in vitro transcription step was treated with portions of GTP (1.25 mM), S-adenosyl methionine, RNAse inhibitor, 2′-O-Methyltransferase and guanylyl transferase are mixed together with reaction buffer (10×, 500 mM Tris-HCl (pH 7.5), 60 mM KCl, 10 mM MgCh). The combined solution was incubated for a range of time at 37° C. for 30 to 90 minutes. Upon completion, aliquots of ATP (2.0 mM), PolyA Polymerase and tailing reaction buffer (lO×, 500 mM Tris-HCl (pH 7.5), 2.5 M NaCl, 100 mM MgCh) were added and the total reaction mixture was further incubated at 37° C. for a range of time from 20 to 45 minutes. Upon completion, the final reaction mixture was quenched and purified accordingly.

### Agarose Gel Electrophoresis:

1% Agarose gels were prepared using 0.5 g Agarose in 50 ml TAE buffer. 1 to 2 g of RNA was treated with 2× Glyoxal gel loading dye or 2× Formamide gel loading dye, loaded on the Agarose gel and run at 130V for 30 or 60 minutes.

### Capillary Electrophoresis (CE)

The standard sensitivity RNA analysis kit was (15 nt) was purchased from Advanced Analytical and used in capillary electrophoresis runs on the Fragment Analyzer instrument with a twelve-capillary array (Advanced Analytical). Upon gel priming, 300 ng of total RNA was mixed with diluent marker at 1:11 (RNA:Marker) ratio and 24 WWL was loaded per well in a 96-well plate. The molecular weight indicator ladder was prepared by mixing 2 pl of the standard sensitivity RNA ladder with 22 WWl diluent marker. Sample injection was at 5.0 kV, 4 seconds and sample separation at 8.0 kV, 40.0 min. A fluorescence-based electropherogram of each sample was processed through the ProSize 2 software (Advanced Analytical), producing tabulated sizes (bp) and abundances (ng/WWl) of fragments present in the sample.

### Digestion Liquid Chromatography Mass Spectrometry (LC/MS)

Probe oligonucleotides were annealed to mRNA transcripts, followed by digestion by RNase H and Shrimp Alkaline Phosphatase. The digestion reaction consisted of 1× RNase H buffer (NEB), RNase H (NEB), Shrimp Alkaline Phosphatase (NEB), annealed mRNA and probe oligonucleotide. The digestion reactions were performed for 40 minutes at 37° C.

Analysis of the mRNA fragment was conducted with an UHPLC-QTOF system (Agilent). Mobile phase A consisted of 100 mM hexafluoroisopropanol and 8.6 mM triethylamine, pH 8.3, and mobile phase B was 100% MeOH. An Agilent InfinityLab C18 2.1×100 mm column was used for all analyses with a flow rate of 0.5 mL/min at 50° C. A gradient from 5% to 23% of mobile phase B was applied over a 12 min period, followed by a 2 min wash step with 50% mobile phase B to elute the RNA fragments. All mass spectra were obtained in the negative ion mode over a scan range of 400-3200 m/z with the following MS settings: drying gas flow, 13 L/min; gas temperature, 350° C.; nebulizer pressure, 10 psi; capillary voltage, 3750 V. The sample data were acquired using the MassHunter Acquisition software (Agilent).

### RNA Sequencing

An oligo was designed comprising a 3′ barcode sequence of 10-15 bases, followed by a poly-A stretch of 25-40 As, with phosphate groups at both the 3′ and 5′ ends to allow ligation to the mRNA transcript and to prevent self-ligation, respectively. First, the mRNA transcripts were treated with rSAP to remove their 5′ phosphate groups in order to prevent the ligation of the oligo to the 5′ end of the mRNA transcript. Using T4 RNA Ligase, the oligo was ligated to the mRNA transcript, yielding an HO-mRNA transcript-Barcode-PolyA-P04 construct. A second rSAP treatment step removed the 3′ phosphate from the above construct in preparation for Nanopore sequencing (MinION, Oxford Nanopore).

For Nanopore sequencing, the HO-mRNA transcript-Barcode-PolyA-OH construct was annealed and ligated to sequencing adaptors and tethered according to the manufacturer's protocol before being loaded into the Nanopore cell chip. Once loaded, the sample was pulled through the nanopores in a 3′ to 5′ direction. Following completion of sequencing, reads were parsed for those containing a portion of the barcode, and the bases following that region collected and analyzed.

### Example 2: Presence of an rrnB Terminator t1 Signal Causes Premature Termination of an mRNA Transcript

This example illustrates how the inadvertent presence of a termination signal in a codon-optimized DNA sequence of a protein coding sequence of interest can result in premature termination of in vitro transcription, thus resulting in a heterogeneous population of mRNA transcripts of which only a portion includes the full-length protein coding sequence.

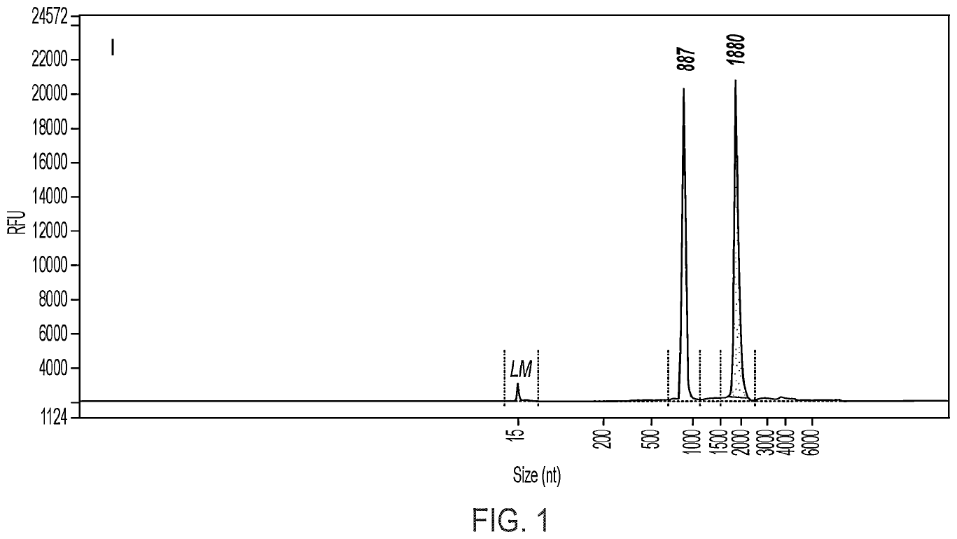

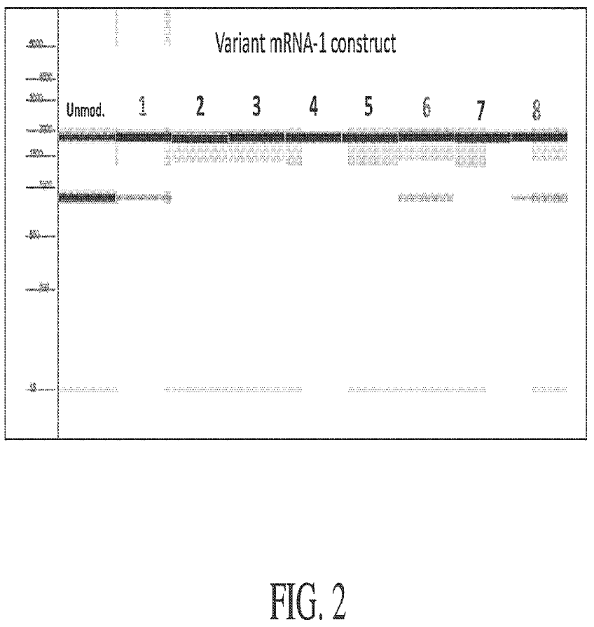

A plasmid with a DNA sequence encoding a codon-optimized protein coding sequence of interest (mRNA-1) operably linked to RNA polymerase promoter was used for in vitro transcription of mRNA transcripts using SP6 RNA polymerase as described in Example 1. The size of the mRNA transcripts was assessed by capillary electrophoresis (CE) (FIG. 1). Full-length mRNA-1 transcripts were ˜1900 nucleotides in length. Approximately 45% of the mRNA-1 transcripts were truncated transcripts ˜900 nucleotides in length (FIG. 1).

The presence of the E. coli rrnB terminator t1 signal consensus sequence TATCTGTT has been reported to cause pausing or termination of both SP6 and T7 RNA polymerases (Kwon & Kang 1999, The Journal of Biological Chemistry, 274:41, pp 29149-29155; Sohn & Kang, 2005, PNAS, 102:1, pp. 75-80). An analysis of the mRNA-1 protein coding sequence found that it includes the consensus rrnB terminator t1 signal starting at nucleotide 796.

In the wildtype rrnB terminator t1 signal, the consensus sequence TATCTGTT is immediately followed by a GTTTGTCGTG sequence (SEQ ID NO: 37). When assessing termination efficiency of variant rrnB1 terminator t1 signal sequences, Kwon & Kang (ibid.) included at least three T bases in the 5 nucleotides immediately 3′ of the TATGTCTT consensus sequence in all but one of the variant terminator t1 signals tested. When this region was deleted, 0% termination efficiency was observed, suggesting that this downstream T-rich sequence is required for termination at the rrnB terminator t1 signal. However, the consensus rrnB terminator t1 signal in mRNA-1 is not followed by a downstream T-rich sequence (instead, it is found within the following sequence: 5′-GCTATCTGTTCATCA-3′. SEQ ID NO: 29), demonstrating that this is not an essential element of the terminator sequence.

This example demonstrates that the presence of the rrnB terminator t1 signal consensus sequence in an mRNA construct can result in premature termination of mRNA transcripts, even in the absence of a T-rich sequence, leading to a significant reduction in the yield of the desired full-length mRNA transcripts.

### Example 3: Variants of the rrnB Termination t1 Signal Also Lead to Premature Termination of mRNA Transcripts

This example demonstrates that the presence of an rrnB terminator t1 signal having a point mutation at position 1, 6 or 8 relative to the consensus sequence also leads to premature termination during in vitro transcription.

Variants of the mRNA-1 protein coding sequence from Example 2 were produced in which the TATCTGTT consensus termination signal sequence was mutated at a single position to determine which nucleotides within the rrnB termination t1 signal are essential for termination of transcription (see Table 1 below).

The variants were used for in vitro transcription using SP6 RNA polymerase. The size of the mRNA transcripts was determined by capillary electrophoresis as described in Example 1. The band observed at ˜1900 nucleotides represents the full-length mRNA-1 construct. A band at ˜900 nucleotides was observed where the mRNA transcript had been truncated due to premature termination at the variant termination signal. The results of this experiment are shown in a digital gel image generated from quantification of mRNA transcripts by CE (FIG. 2) and summarized in Table 1.

TABLE 1
Construct design and results of truncation analysis

| | | | | | | | | | Results of |
|---|---|---|---|---|---|---|---|---|---|
| | Nucleotide position | | | | | | | | truncation |
| | 1 | 2 | 3 | 4 | 5 | 6 | 7 | 8 | analysis |
| Unmodified | T | A | T | C | T | G | T | T | Truncated |
| | | | | | | | | | at ~900 nt |
| Variant 1 | A/C/G | A | T | C | T | G | T | T | Truncated |
| | | | | | | | | | at ~900 nt |
| Variant 2 | T | C/G/T | T | C | T | G | T | T | No truncation |
| Variant 3 | T | A | A/C/G | C | T | G | T | T | No truncation |
| Variant 4 | T | A | T | A/G/T | T | G | T | T | No truncation |
| Variant 5 | T | A | T | C | A/C/G | G | T | T | No truncation |
| Variant 6 | T | A | T | C | T | A/C/T | T | T | Truncated |
| | | | | | | | | | at ~900 nt |
| Variant 7 | T | A | T | C | T | G | A/C/G | T | No truncation |
| Variant 8 | T | A | T | C | T | G | G | A/C/G | Truncated |
| | | | | | | | | | at ~900 nt |

The mRNA-1 variants still yielded truncated mRNA transcripts if the identity of the nucleotide at position 1, 6 or 8 had been changed relative to the consensus sequence. However, no truncation was observed if the nucleotide at position 2, 3, 4, 5 or 7 had been mutated. Therefore, when screening for termination signals within DNA sequence with a protein coding sequence, both the consensus sequence and sequence variants with point mutations at positions 1, 6 and 8 should be taken into account to avoid premature termination of in vitro transcription.

It was previously known that no termination occurs if C at the fourth position of the TATCTGTT consensus termination signal sequence is replaced with a G (Sohn & Kang, 2005, PNAS, 102:1, pp. 75-80). The finding that no truncation was observed if any of the residues at position 2, 3, 4, 5 or 7 are replaced with another nucleotide not naturally present at that position provides greater flexibility to remove termination signals without altering the protein coding sequence encoded by the DNA sequence.

### Example 4: rrnB Terminator t1 Signals Cause Premature Termination of mRNA Transcripts

This example demonstrates that sites of premature termination in mRNA transcripts produced by in vitro transcription using SP6 RNA polymerase can be predicted by in silico screening for rrnB terminator t1 signals.

Various DNA sequences with codon-optimized protein coding sequences were screened in silico for the presence of the E. coli rrnB terminator t1 signal consensus sequence TATCTGTT or variant sequences that differ from this sequence at position 1, 6 or 8, as determined in Example 3. This analysis was performed to predict the size of truncated mRNA transcripts that would be produced when the polymerase prematurely terminated at these termination signals (see Table 2, below).

To test the in silico prediction for accuracy, the codon-optimized protein coding sequences were transcribed in vitro using SP6/T7 RNA polymerase, and the actual size of the mRNA transcripts was determined by capillary electrophoresis (CE), as described in Example 1. The actual size of any truncated mRNA transcripts was compared with the size predicted by the in silico analysis (see Table 2).

TABLE 2
Predicted and experimentally-determined truncated
mRNA transcript size

| | | Truncated mRNA | |
|---|---|---|---|
| | Identified | transcript size | |
| | termination | In silico | Determined |
| Construct | signal | prediction | by CE |
| mRNA-1 | TATCTGTT | 804 | 887 |
| mRNA-2 | TATCTGTG | 403 | 438 |
| mRNA-3 | TATCTGTC | 1134 | 1233 |
| mRNA-4 | TATCTGTC | 1014 | 1085 |
| mRNA-5 | TATCTGTT | 432 | 457 |
| mRNA-6 | TATCTGTC | 1134 | 1120 |
| mRNA-7 | TATCTGTC | 1134 | 1219 |
| mRNA-8 | TATCTGTT | 1014 | 1050 |
| mRNA-9 | TATCTGTC | 1014 | 1071 |
| mRNA-10 | TATCTGTC | 286 | 350 |
| mRNA-10 | TATCTATT | 1332 | 1384 |
| mRNA-11 | TATCTGTT | 1219 | 1309 |

Truncation of the mRNA transcripts was observed at all identified terminator signals. The predicted size of the truncated mRNA transcripts based on the identification of rrnB terminator t1 signals correlated well with the experimentally determined size of the truncated mRNA transcripts.

### Example 5: mRNA Transcripts Produced by SP6 RNA Polymerase do not Contain Duplex RNAs

This example demonstrates that the mRNA transcripts of SP6 RNA polymerase, unlike mRNA transcripts synthesized by T7 RNA polymerase, do contain RNA duplexes.

The mRNA transcripts synthesized with T7 RNA polymerases are typically contaminated with RNAs longer and shorter than the desired transcript (see WO 2018/157153). Very short transcripts are commonly referred to “shortmers” and typically have to be removed by extensive purification of in vitro transcribed mRNA.

Elongated sequences are thought to be generated by non-templated additions of nucleotides at the end of the template-encoded mRNA transcript after a termination signal. The additional nucleotides are commonly referred to as “runoff”. Further extension can occur when the 3′ end of the runoff has sufficient complementarity to bind to itself or a second mRNA molecule to form extendible intra- or intermolecular duplexes, respectively (Gholamalipour et al. 2018, Nucleic Acids Research 46:18, pp 9253-9263).

mRNA transcripts were produced by in vitro transcription from four different template plasmids using either SP6 RNA polymerase or T7 RNA polymerase, as described in Example 1. Each template plasmid encoded an mRNA transcript encoding the same protein. The presence of RNA duplexes was detected by dot blot analysis performed with the anti-dsRNA monoclonal antibody J2, as described in Baiersdôrfer et al. 2019, Molecular Therapy: Nucleic Acids, 15: 26-35.

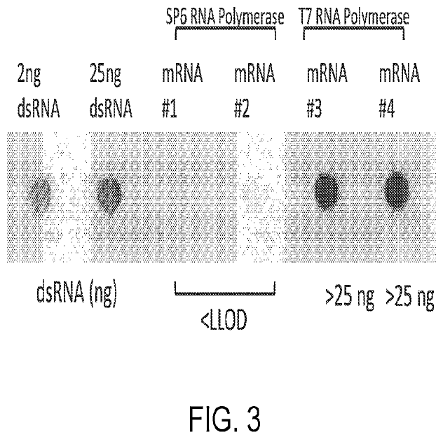

2 μl of each sample of in vitro transcribed mRNA, corresponding to 200 ng total mRNA per dot, was spotted on a positively charged nylon membrane. A control sample of dsRNA was spotted at 2 ng and 25 ng per dot. For the detection of dsRNA, the membrane was incubated with anti-dsRNA murine monoclonal antibody J2. An anti-mouse IgG antibody conjugated to horse radish peroxidase was used for detection. The resulting dot blot is shown in FIG. 3. As can be seen from this figure, no double-stranded RNA was detected in mRNA transcripts synthesized with SP6 RNA polymerase, whereas copious amounts of double-stranded RNA was detected in samples prepared with T7 RNA polymerase.

mRNA transcripts synthesized by SP6 RNA polymerase do not form intra- or intermolecular duplexes.

### Example 6: mRNA Transcripts Produced by T7 RNA Polymerase and by SP6 RNA Polymerase are Extended by Non-Templated Elongation

This example demonstrates that mRNA transcripts synthesized by both T7 and SP6 RNA polymerase are elongated by “runoff” transcription.

The use of SP6 RNA polymerase for in vitro transcription avoids the formation of shortmers in mRNA transcripts as commonly observed with T7 RNA polymerase (see WO 2018/157153). mRNA transcripts synthesized by SP6 RNA polymerase typically appear to be more homogenous in size.

To determine whether SP6 RNA polymerase, like T7 RNA polymerase, continues to elongate mRNA transcripts in a non-template-mediated fashion after encountering a termination signal (“runoff” transcription), a set of probe oligonucleotides was designed which bind to the 3′ end of the mRNA transcripts as encoded by the templated sequence. The probe oligonucleotide were RNA-DNA gap oligonucleotides synthesized by Integrated DNA Technologies (Coralville, IA). Their sequence and sugar modifications are shown in Table 3. 2′-O-methyl ribose modified RNA nucleotides are shown with a “m” preceding the corresponding base, and the DNA nucleotides are italicized.

TABLE 3
Probe oligonucleotides

| | | Digestion |
|---|---|---|
| # | Nucleotide Sequence | product |
| 1 | 5′ mGmAmUmG CA A CmUmUmAmAmUmUmUmU | CAUCAAGCU |
| | (SEQ ID NO: 30) | |
| 2 | 5′ mAmGmCmUmUmGmA T G C AmAmCmUmUmAmAmU | UCAAGCU |
| | (SEQ ID NO: 31) | |
| 3 | 5′ mAmGmCmUmU G A T GmCmAmAmCmUmUmA | AAGCU |
| | (SEQ ID NO: 32) | |
| 4 | 5′ mAmGmC T T G AmUmGmCmAmAmCmUmUmA | GCU |
| | (SEQ ID NO: 33) | |
| 5 | 5′ mAmGmCmUmUmGmA T G C AmAmCmU | UCAAGCU |
| | (SEQ ID NO: 34) | |
| 6 | 5′ mCmUmUmGmA T G C AmAmC | UCAAGCU |
| | (SEQ ID NO: 35) | |

RNaseH was added to digest the DNA/RNA hybrids, leaving only the fragments of the mRNA transcript that are 3′ of the templated sequence. The size of the 3′ end digestion products of the mRNA transcripts was determined by liquid chromatography mass spectrometry (LC/MS), as described in Example 1. Of the six oligonucleotides that were tested, probe oligonucleotide #1 yielded the longest expected 3′ digestion product (CAUCAAGCU) and was selected for further experiments.

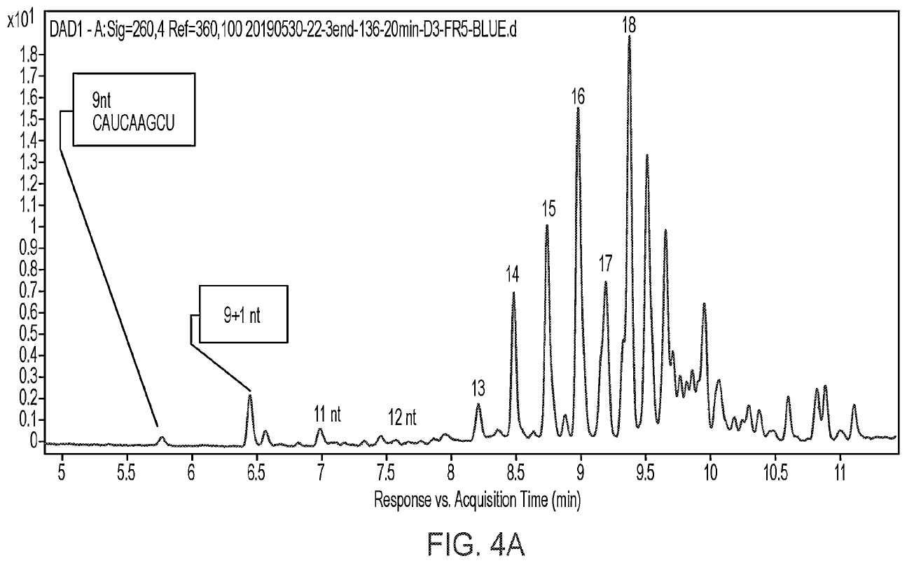

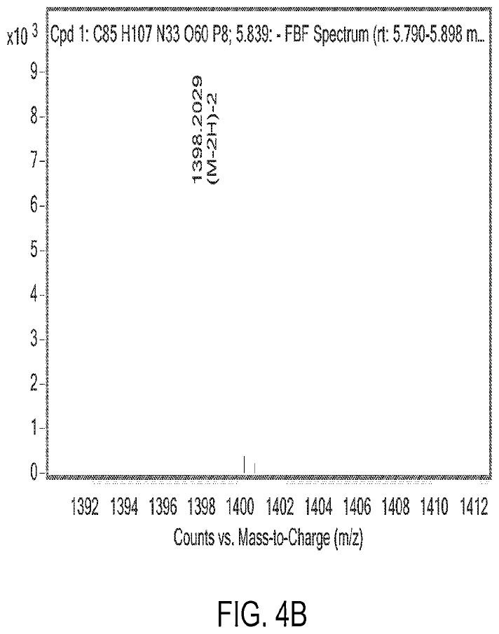

The results are shown in FIG. 4A. A 9 nucleotide 3′ digestion product (CAUCAAGCU) was obtained with probe oligonucleotide #1 if the SP6 mRNA transcript terminated at the end of the templated sequence (i.e. where there was no runoff elongation). The identity of this digestion product was confirmed by mass spectrometry, as described in Example 1. The results of the mass spectrometry analysis are shown in FIG. 4B. Longer 3′ digestion products were obtained where runoff elongation of the mRNA product had occurred.

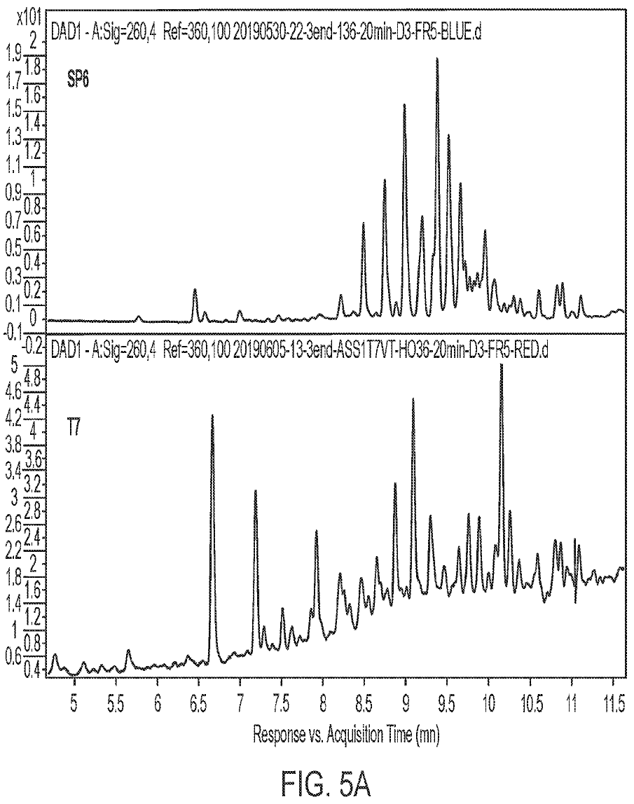

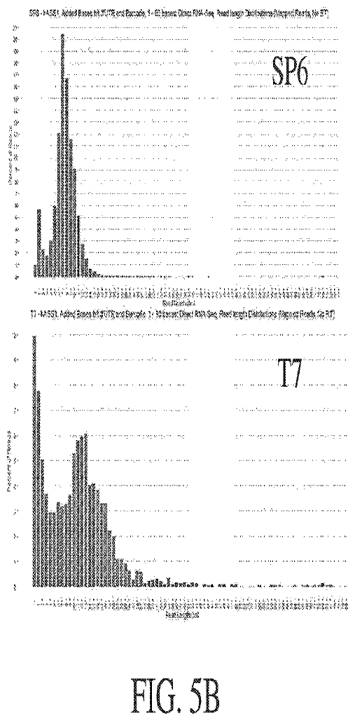

The experiment was repeated with T7 RNA polymerase. The results of LC/MS analysis of the 3′ digestion products of mRNA transcribed by SP6 and T7 RNA polymerases are compared in FIG. 5A. The number of bases added to the 3′ by runoff elongation was also determined by sequencing the T7 RNA polymerase and SP6 RNA polymerase mRNA transcripts (FIG. 5B). mRNA transcripts synthesized by SP6 RNA polymerase had shorter runoff sequences relative to mRNA transcripts synthesized by T7 RNA polymerase, but also yielded a lower percentages of mRNA transcripts with no additional run-off sequences.

These data demonstrate that non-templated elongation of in vitro synthesized mRNA transcripts occurs when both SP6 RNA polymerase as well as by T7 RNA polymerase is used.

### Example 7: Inclusion of Termination Signals at the 3′ End Prevents Undesired Elongation of mRNA Transcripts Synthesized from a Linearized Plasmid

This example demonstrates that the addition of one or more termination signals at the 3′ end of a DNA sequence encoding an mRNA transcript reduces undesired elongation of mRNA transcribed from a linearized plasmid.

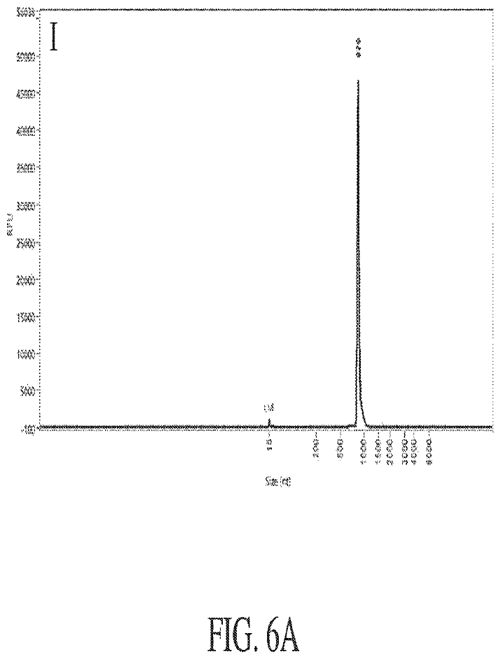

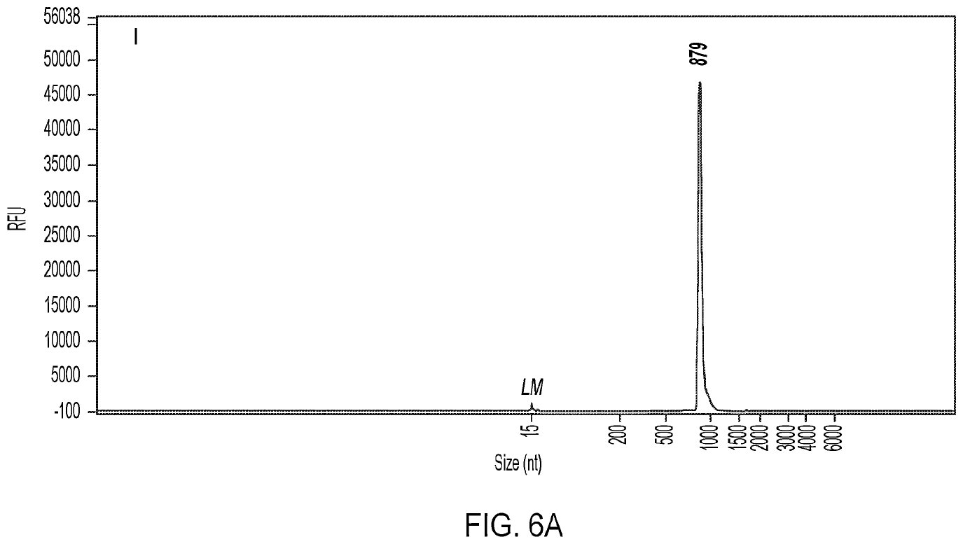

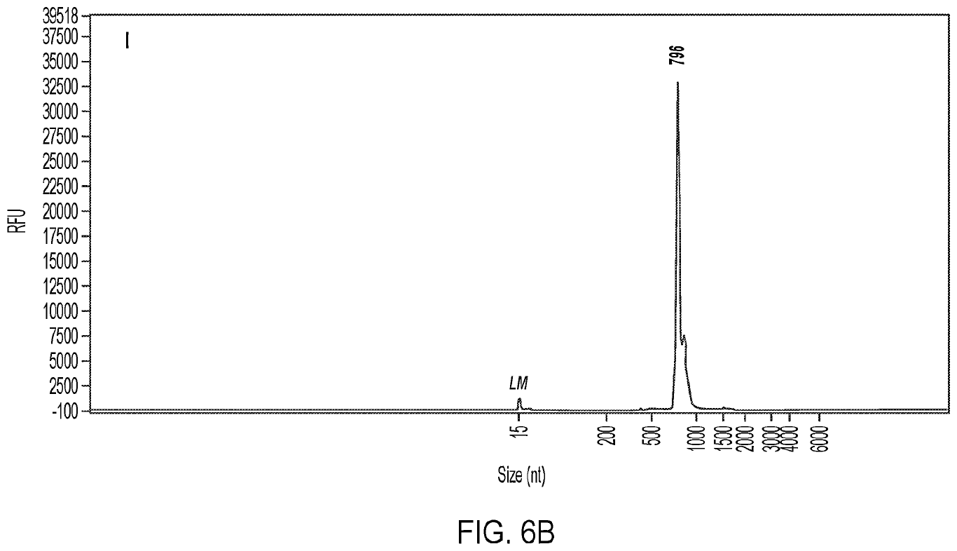

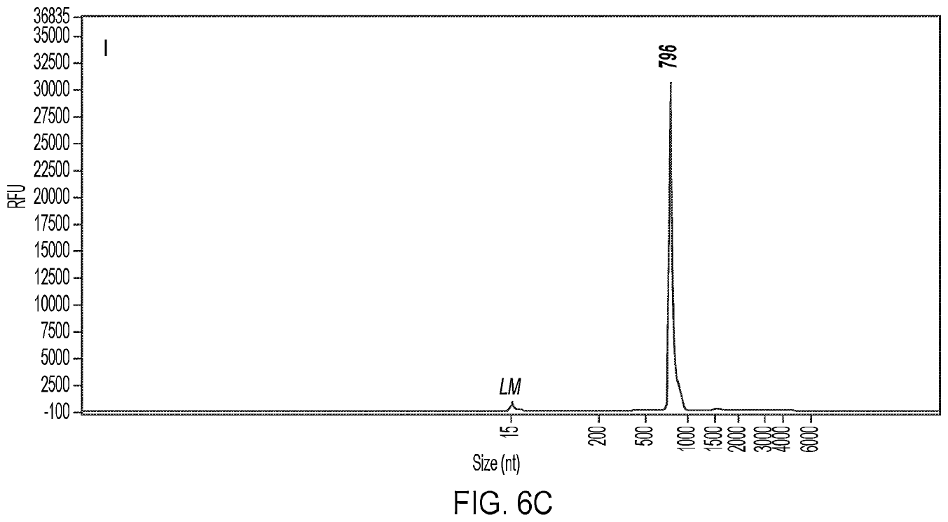

A DNA sequence encoding an mRNA transcript (mRNA-12) was operably linked to an SP6 RNA polymerase promotor by insertion into a plasmid using standard molecular biology procedure. The resulting plasmid was used for in vitro transcription either with or without prior linearization. Linearization was performed by cutting the plasmid with a sequence specific restriction enzyme 880 bp downstream from the transcription start site. As shown in FIG. 6A, the linearized plasmid yielded a single 879 nt long mRNA transcript, as determined by capillary electrophoresis, as described in Example 1.

To determine whether insertion of a termination sequence could result in effective termination of transcription at the end of the DNA sequence, two modified plasmids were prepared. Plasmid 1 included a single rrnB termination t1 signal

(TTTTATCTGTTTTTTTTTT (SEQ ID NO: 14))

at the 3′ end of the DNA sequence encoding the mRNA transcript. Plasmid 2 contained two copies the same termination signal at the 3′ end of the DNA sequence

(TTTTATCTGTTTTTTTTTTTTTATCTGTTTTTTTTT (SEQ ID NO:
12)).

The unmodified plasmid and modified plasmids 1 and 2 were linearized and used as templates for in vitro transcription using an SP6 RNA polymerase, and the size of the mRNA transcripts is determined by capillary electrophoresis, as described in Example 1.

As shown in FIG. 6B, plasmid 1 yielded a shorter mRNA transcript of 796 nt in length, demonstrating that termination occurred at the newly added termination sequence. However, the termination signal was not completely effective in stalling the polymerase, as evidenced by a second peak close to the first, suggesting that there are a relatively high number of instances in which the RNA polymerase does not termination directly at the termination signal and instead continues to transcribe for a short distance before terminating transcription. This second peak was not visible for mRNA transcripts produced from plasmid 2 (see FIG. 6C), demonstrating that the inclusion of two termination signals in tandem separated by just 10 base pairs was efficient in prevent undesired elongation of mRNA transcripts.

### Example 8; Inclusion of Termination Signals at the 3′ End Prevents Undesired Elongation of mRNA Transcripts Synthesized from Supercoiled Plasmid DNA

This example demonstrates that the addition of one or more termination signals at the 3′ end of a DNA sequence encoding an mRNA transcript obviated the need to linearize a circular nucleic acid vector prior to in vitro transcription.

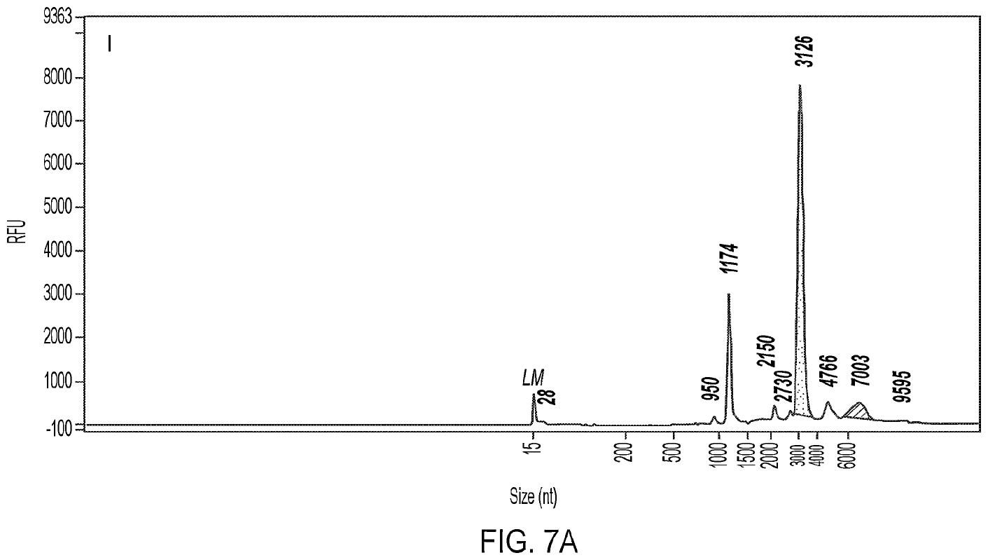

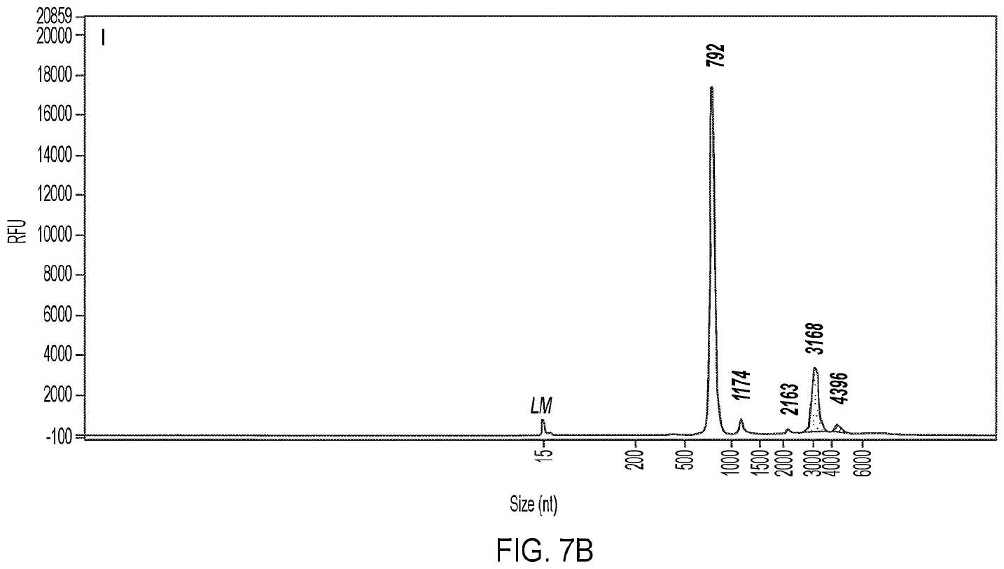

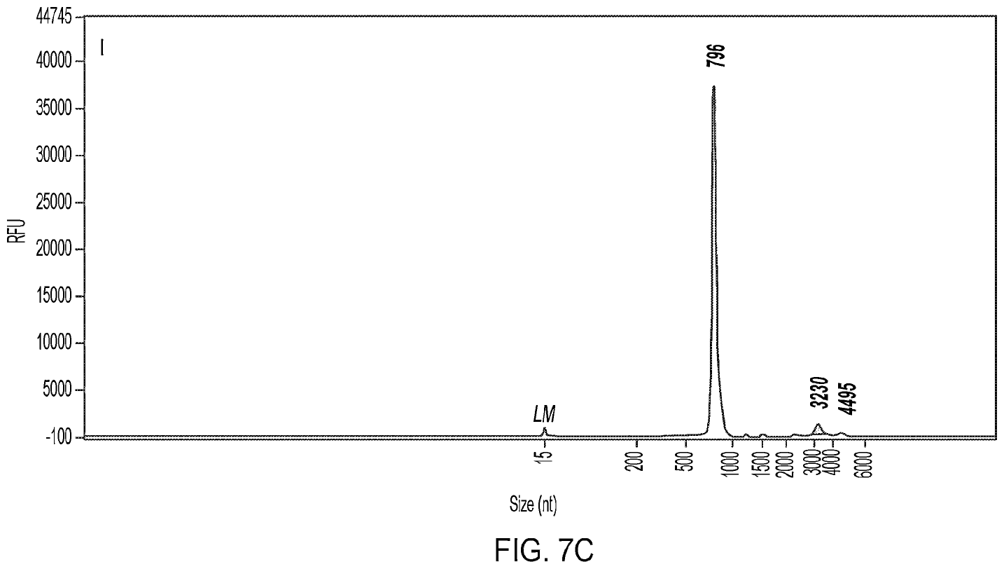

To determine whether linearization was required if a termination signal was included at the 3′ end of the DNA sequence encoding the mRNA transcript, the experiment of example 7 was repeated without linearization of the plasmid prior to in vitro transcription. When supercoiled plasmid DNA was used for in vitro transcription of the unmodified plasmid, multiple new peaks were visible during capillary electrophoresis of the resulting mRNA transcripts (FIG. 7A). The largest peak (representing ˜55% of the total mRNA transcripts) corresponded to mRNA transcripts of ˜3126 nt to ˜3230 nt in length. Closer inspection of the nucleotide sequence of the plasmid identified a termination signal (CATCTATT) downstream of the DNA sequencing encoding the mRNA transcript. Based on the nucleotide sequence analysis, the predicted size of the mRNA transcript would be expected to be 3306 nt and therefore correlated well with the observed peak size at ˜3126 nt to ˜3230 nt. This observation indicates that the RNA polymerase continued to transcribe the supercoiled plasmid DNA until it encountered a termination signal already present in the plasmid backbone, at which point transcription was terminated, incidentally confirming that the inclusion of a termination signal at the end of the DNA sequence encoding the desired mRNA transcript should obviate the need for plasmid linearization prior to in vitro transcription. The presence of multiple smaller peaks corresponding to even larger transcripts suggests that at least a portion of RNA polymerases in the reaction mixture transcribe the plasmid template multiple times before the reaction was terminated. In contrast, when plasmid 1 was used as the template, the presence of the termination signal TATCTGTT resulted in more effective termination of transcription, with ˜70% of the mRNA transcripts having a size of ˜792 nt (FIG. 7B). With plasmid 2, which contains two TATCTGTT termination signals in tandem separated by just 10 base pairs, the percentage of correctly-terminated mRNA transcripts was further improved to ˜95% (FIG. 7C).

This example demonstrates that the presence of one or more termination signals at the 3′end of a DNA sequence encoding the mRNA transcripts obviates the need for linearization of the plasmid comprising the template, as mRNA synthesis is terminated predominately at the end of the DNA sequence. Given the length of the mRNA transcripts, runoff transcription also appears to be prevented by the presence of a termination signal. In particular, the inclusion of two consensus termination signals separated by just 10 base pairs can lead to highly efficient termination of transcription, preventing runoff transcription and obviating the need for plasmid linearization.

### Example 9: In Vitro Transcription at a Temperature Higher than 37° C. Improves Termination

This example demonstrates that the likelihood of termination to occur at one or more termination signals is higher if the in vitro transcription reaction is performed at a temperature higher than 37° C.

In order to determine if the percentage of correctly-terminated mRNA transcripts could be improved further, the experiment described in Example 8 was repeated with plasmid 2 but at different temperatures. Using an SP6 RNA polymerase and, aside from the temperature, reaction conditions identical to those described in Example 1, supercoiled plasmid 2 was used as a template for an in vitro transcription reaction. The size of the resulting mRNA transcripts was determined by capillary electrophoresis, also as described in Example 1.

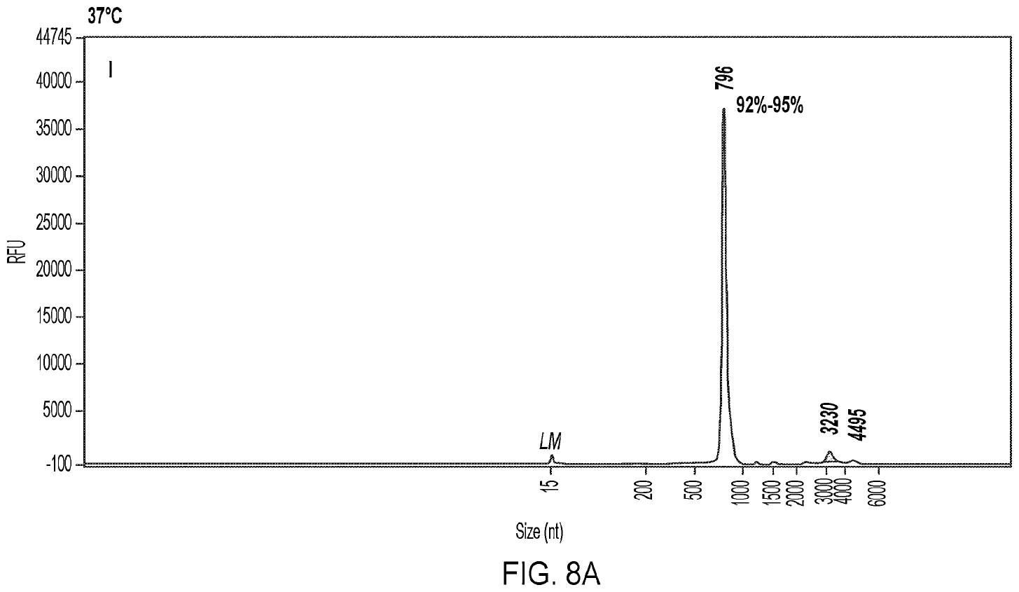

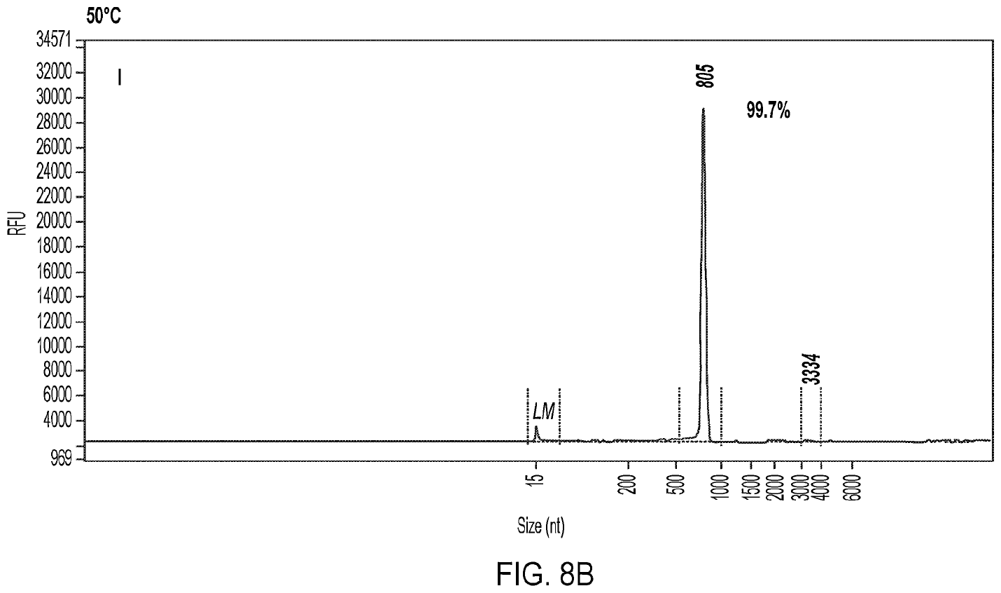

The reaction temperature was controlled by performing the in vitro transcription reaction in Eppendorf tubes placed in a block heater. At the previously employed temperature of 37° C., 92%-95% of the mRNA transcripts obtained from supercoiled plasmid 2 were correctly terminated, as shown in FIG. 8A. The percentage of correctly-terminated plasmids further increased as the temperature at which the in vitro transcription reaction was performed was increased to 43° C., 50° C. or 55° C. The yield of correctly-terminated mRNA transcripts peaked at 50° C. As shown in FIG. 8B, at that temperature 99.7% of the mRNA transcripts obtained from plasmid 2 were correctly terminated. Only minimal degradation was observed at 50° C.

Employing a supercoiled plasmid with one or more termination signals and performing the in vitro transcription reaction at a temperature higher than 37° C. resulted in reaction conditions that maximize the yield of correctly-terminated mRNA transcripts without the need for plasmid linearization.

### Example 10: Inclusion of More than Two Termination Signals at the 3′ End Results in Correctly-Terminated mRNA Transcripts at 37° C.

This example demonstrates that a yield of correctly-terminated mRNA transcripts approaching 100% can be reached when in vitro transcription is performed at 37° C., if more than two copies of a termination signal are present at the 3′ end of a DNA sequence encoding the mRNA transcript. This makes it possible to perform an in vitro transcription reaction at conventional conditions without the need to linearize a circular nucleic acid vector prior to performing the reaction.

To investigate the effect of insertion of more than two termination signals into the template DNA plasmid, further modified plasmids encoding mRNA-12 were prepared. Plasmids 1 and 2 were prepared as described in Example 7. Plasmid 3 contained three copies of the rrnB termination t1 signal at the 3′ end of the DNA sequence encoding the mRNA transcript, creating the following terminator sequence:

(SEQ ID NO: 13)
TTTTATCTGTTTTTTTTTTTTTATCTGTTTTTTTTTTTTTATCTGTTTTT
TTTT.

While the terminator sequences in plasmids 1 and 2 were inserted directly after the protein coding region of mRNA-12, the terminator sequence in plasmid 3 was inserted after the 3′ UTR region. Therefore, when plasmid 3 is used as a DNA template for in vitro transcription the correctly-terminated transcripts are longer (˜880 nucleotides in length) than those produced when plasmid 1 or 2 is used (˜780 nucleotides in length).

The experiment of Example 9 (no linearization of the DNA plasmids, in vitro transcription performed at 37° C. and at 50° C.) was repeated for unmodified plasmid, and modified plasmids 1, 2 and 3. The reaction temperature was controlled by performing the in vitro transcription reaction in Eppendorf tubes placed in a thermocycler.

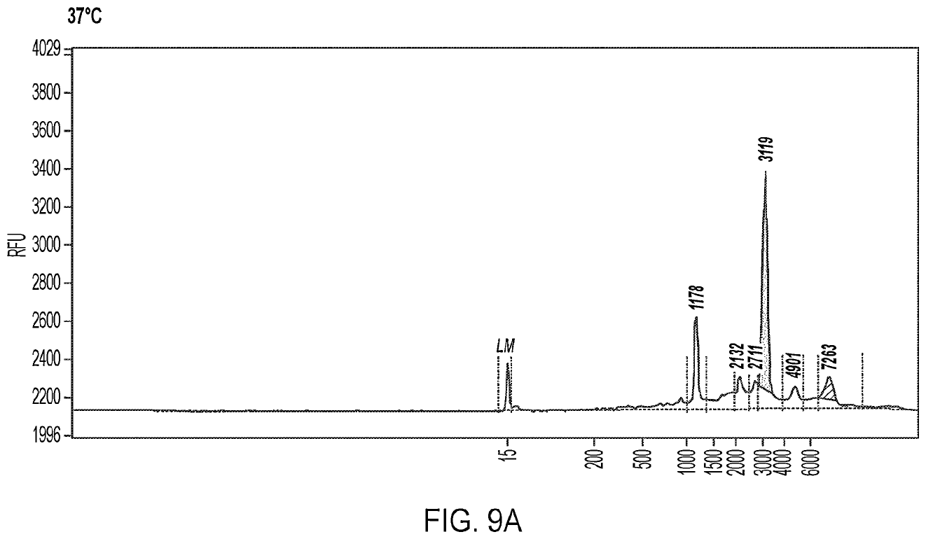

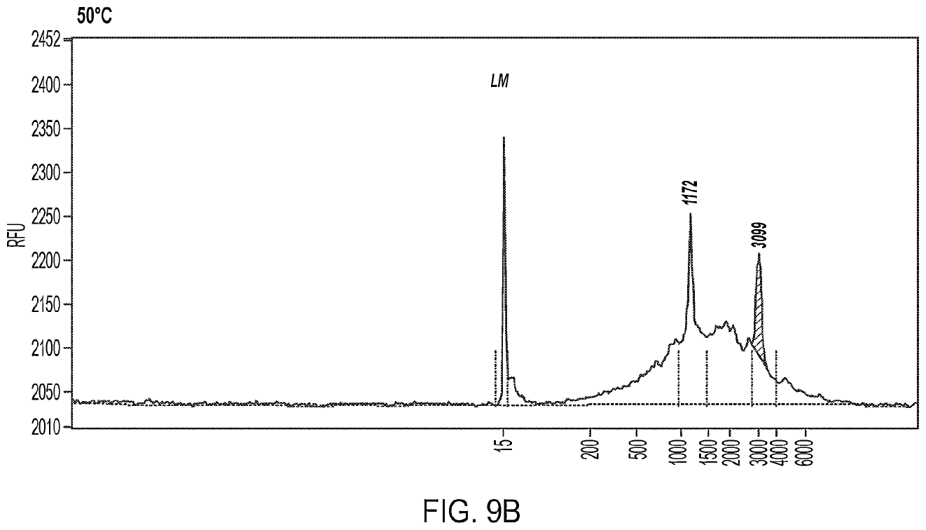

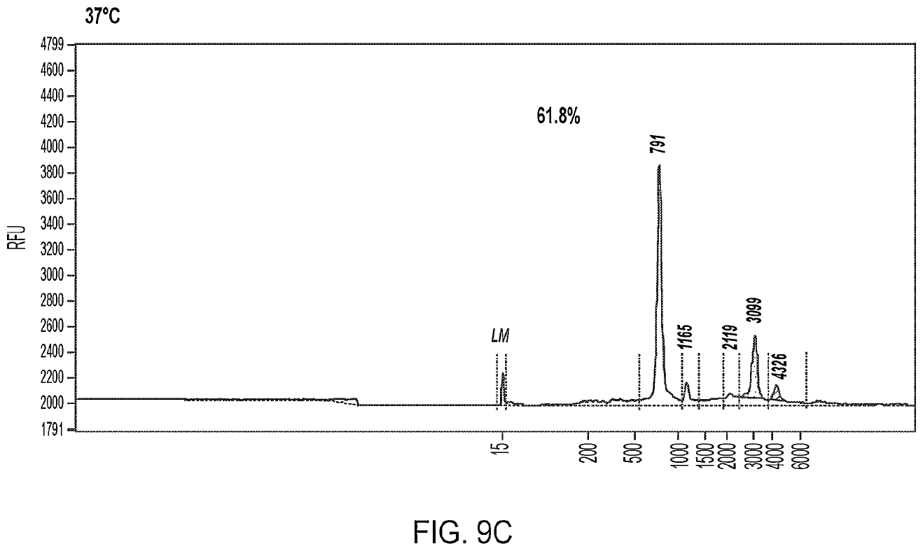

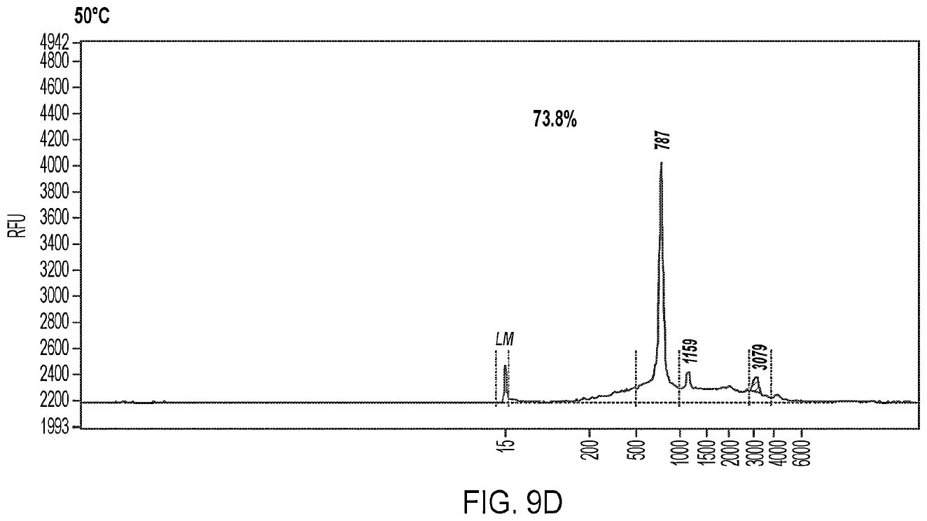

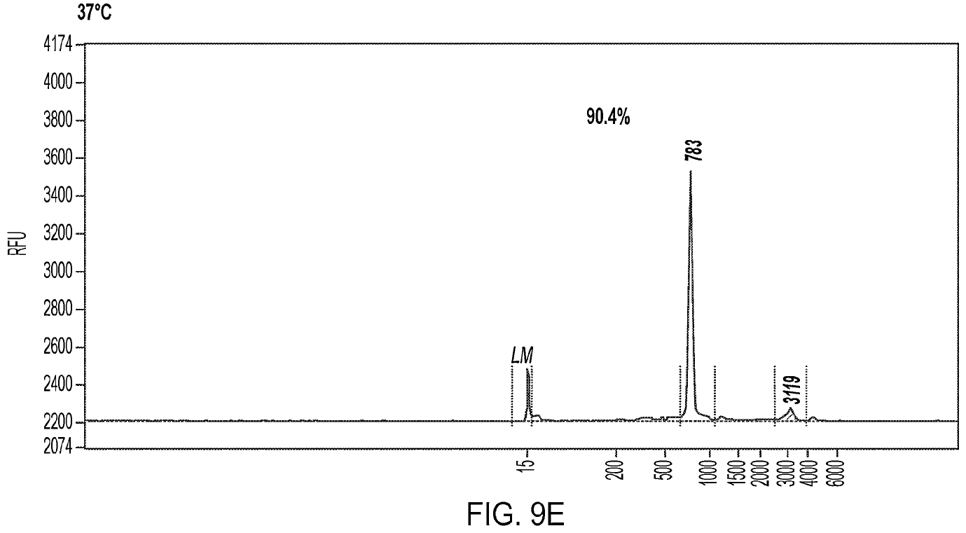

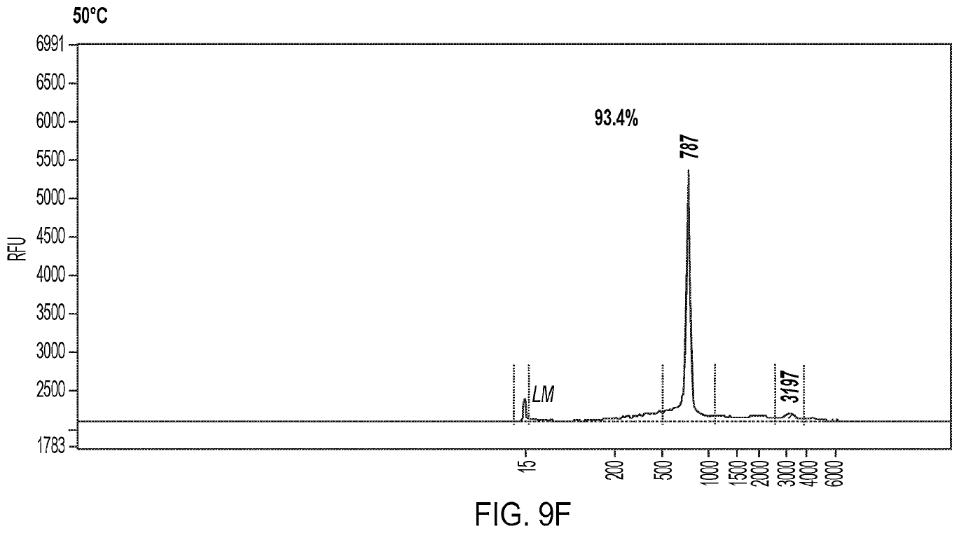

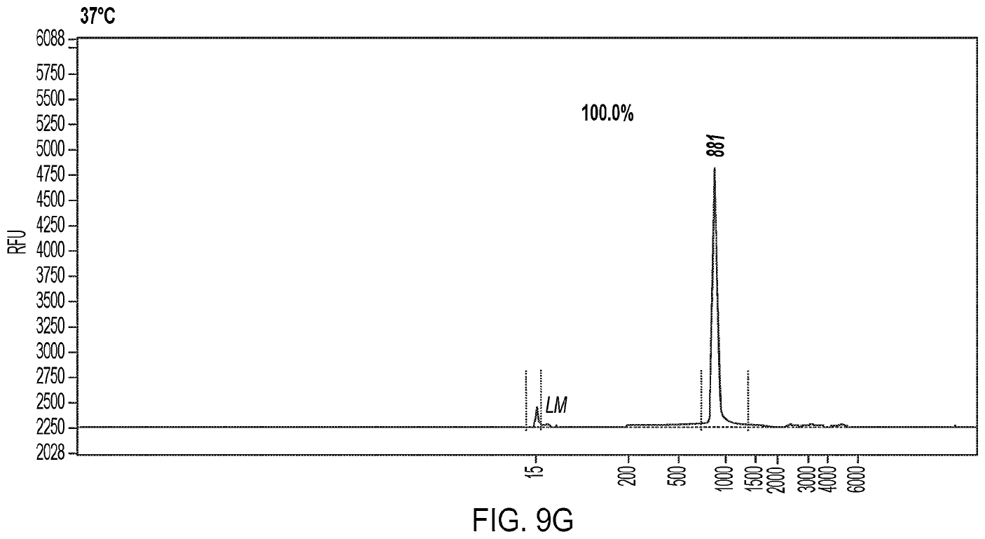

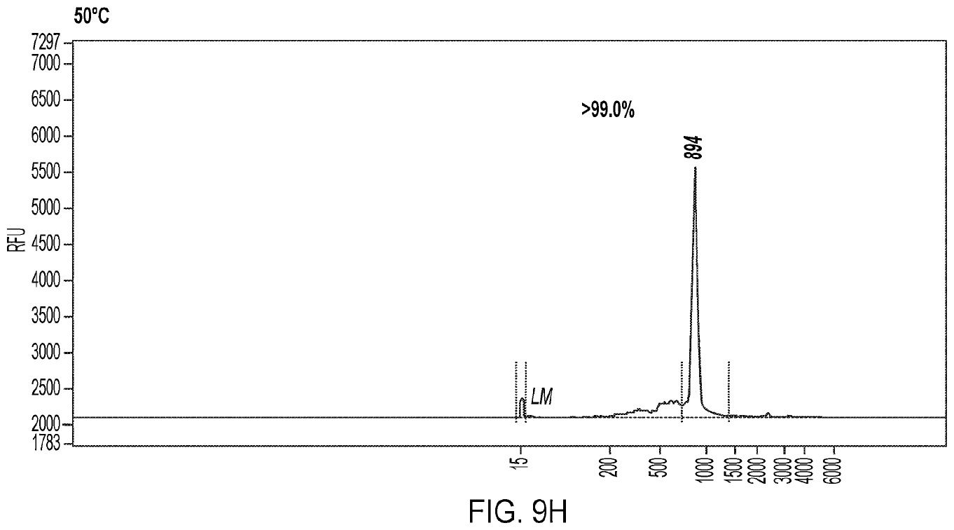

Much like in Example 8, the largest peak observed for in vitro transcription of unmodified plasmid at 37° C. corresponded to mRNA transcripts ˜3119 nt in length (FIG. 9A). This again indicates that the RNA polymerase continued to transcribe the supercoiled plasmid DNA until it encountered a termination signal already present in the plasmid backbone, at which point transcription was terminated. When in vitro transcription was performed at 50° C., degradation was observed, leading to a smaller peak corresponding to mRNA transcripts of ˜3099 nt in length relative to equivalent peak for the 37° C. transcription reaction (FIG. 9B).

When plasmid 1 was used as the template, the presence of the termination signal resulted in more effective termination of transcription, with ˜62% and ˜74% of the mRNA transcripts having a size corresponding to the full-length mRNA-12 transcript for transcription reactions performed at 37° C. and 50° C., respectively (FIGS. 9C and 9D). With plasmid 2, which includes two termination signals in tandem, the percentage of correctly terminated mRNA-12 transcripts was further increased, reaching ˜90% and ˜93% for transcription reactions performed at 37° C. and 50° C., respectively (FIGS. 9E and 9F). The proportion of correctly-terminated transcripts was increased even further for plasmid 3, which includes three termination signals in tandem, reaching a yield approaching 100% for the transcription reaction carried out at 37° C. and >99.0% for 50° C. (FIGS. 9G and 9H). As for unmodified plasmid, significant degradation of the mRNA transcripts was observed for the transcription reactions performed at 50° C. using plasmids 1, 2 or 3 as the DNA template. The fact that significant degradation was observed for the reactions performed at 50° C. in this experiment but not in the experiment described in Example 9 suggests that the reaction temperature in Example 9 may not have been consistently maintained at 50° C. (e.g., because part of the Eppendorf tube is exposed to the ambient air which has a much lower temperature than the heating block itself). This suggests that reaction conditions may need to be optimized to minimize degradation at temperatures higher than 37° C.

This example further confirms that increasing the number of termination signals at the 3′end of a DNA sequence encoding the mRNA transcripts can obviate the need for linearization of the plasmid comprising the template, as mRNA synthesis is terminated predominately at the end of the DNA sequence. It also shows that where one or two termination signals have been included in series, the yield of correctly-terminated mRNA transcripts can be improved by performing the transcription reaction at a higher temperature, although care must be taken to minimize mRNA degradation. The inclusion of more than two termination signals in series allowed a yield of correctly-terminated mRNA transcripts approaching 100% to be reached, demonstrating that termination efficiency can be maximized for such DNA plasmid templates when the transcription reaction is performed at 37° C.

### Example 11: mRNA Transcribed from Supercoiled Plasmid DNA Having a 3′ Termination Signal is Effectively Expressed In Vitro

This example demonstrates that comparable levels of protein expression can be achieved for mRNA transcribed from supercoiled plasmid DNA having a 3′ termination signal and mRNA transcribed from a linearized plasmid. This example therefore provides further evidence that inclusion of a 3′ termination signal obviates the need to linearize a circular nucleic acid vector prior to in vitro transcription.

Protein expression levels were determined for mRNA-12 transcripts prepared by in vitro transcription from supercoiled plasmid 3 as described in example 10 (containing three copies of the rrnB termination t1 signal at the 3′ end of the DNA sequence encoding the mRNA transcript) and for equivalent mRNA-12 transcripts prepared by in vitro transcription from a linearized unmodified plasmid (containing no termination signals).

Protein expression levels were evaluated using a cell-free translational system (CFTS). The CFTS is a useful tool to screen the expression of mRNA constructs in a high-throughput fashion, without requiring the maintenance of cell cultures or the use of transfection agents. The core component of the CFTS is a cytoplasmic extract generated from HeLa cells, containing the necessary machinery required to express protein (Mikami et al. 2005, Protein Expression and Purification, 46, 348-357). Through adjustments of supplementary reaction components, primarily Mg²⁺ and K⁺ levels, protein expression is optimized for the protein of interest. The CFTS reaction conditions and components used in this example have been optimized for expression of the protein encoded by the mRNA-12 transcript.

Two separate CFTS reaction mixtures were prepared for each mRNA-12 transcript. The CFTS reaction mixtures contained 325 fmol mRNA-12 transcript, 40% (v/v) HeLa cytoplasmic extract (20 mg/ml total protein), 27 mM HEPES (pH 7.5), 140 mM KOAc, 1.2 mM Mg(OAc)₂, 16 mM KCl, RNAse Inhibitor (lU/Wu̧WL), 1 mM DTT, 1.2 mM ATP, 125 pM GTP, 30 pM amino acid mix, 300 u̧M spermidine, 18 mM creatine phosphatase, 60 Wu̧g/mL creatine kinase and 90 Wu̧g/mL calf-liver tRNA in a 65 u̧L reaction volume.

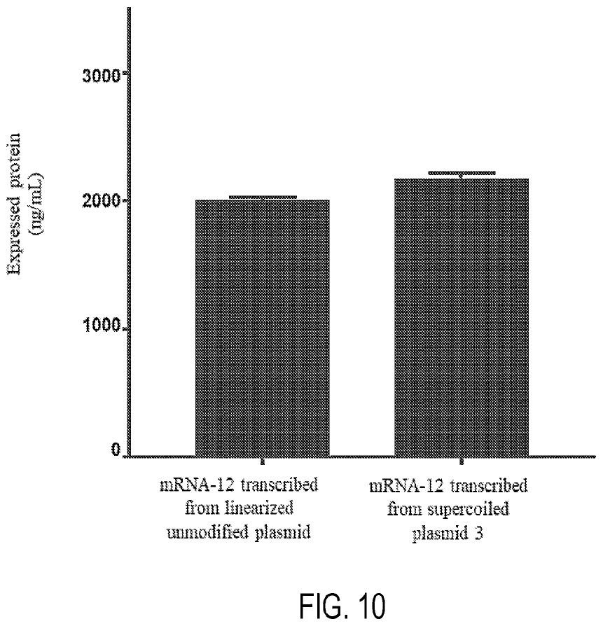

The reaction mixtures were incubated for two hours at 25° C. After this, the reaction mixtures were stored at −80° C. until protein expression levels were determined by ELISA. The results of this analysis are provided in FIG. 10. FIG. 10 shows that comparable or even slightly higher levels of protein expression can be achieved for mRNA-12 transcribed from supercoiled plasmid 3 than for mRNA-12 transcribed from linearized unmodified plasmid.

These data confirm the inventors' finding that the addition of a termination sequence is effective in terminating transcription of the accordingly modified DNA template by an RNA polymerase so that it is no longer necessary to linearize the plasmid comprising the DNA template prior to in vitro transcription. mRNA produced from a super-coiled DNA template having a 3′ termination signal therefore can replace mRNA provided from a linearized plasmid in an existing process for manufacturing mRNA.

The plasmid linearization step typically involves incubation with a restriction enzyme. Removing this step can therefore result in considerable cost savings in the production of mRNA, in particular when done at a large scale to manufacture mRNA as a drug product.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method for preparing an optimized DNA sequence encoding a protein as a template for in vitro transcription, said method comprising:
a. providing a DNA sequence that comprises a protein coding sequence;
b. determining the presence of a termination signal in the DNA sequence, wherein the termination signal has the following nucleic acid sequence: 5′-X₁ATCTX₂TX₃-3′, wherein X₁, X₂ and X₃ are independently selected from A, C, T or G; and
c. if one or more termination signal is present, modifying the DNA sequence by replacing one or more nucleic acids at any one of position 2, 3, 4, 5 and 7 of said termination signal(s) with any one of the other three nucleic acids to generate the optimized DNA sequence, wherein, if required, the one or more replacement nucleic acids are selected to preserve the amino acid sequence of the protein encoded by the protein coding sequence.

2. (canceled)

3. The method of claim 1, wherein the DNA sequence further comprises a first nucleic acid sequence encoding a 5′ UTR and/or a second nucleic acid sequence encoding a 3′ UTR.

4. The method of claim 1, wherein the 5 nucleotides immediately 3′ of the termination signal in the DNA sequence do not comprise 3 or more T nucleotides.

5. The method of claim 1, wherein the method further comprises a step of modifying the DNA sequence relative to a wildtype DNA sequence encoding the same protein sequence to optimize:
a. elements relevant to mRNA processing and stability, wherein the elements relevant to mRNA processing or stability include cryptic splice sites, mRNA secondary structure, stable free energy of mRNA, repetitive sequences, and RNA instability motifs; and/or
b. elements relevant to translation or protein folding, wherein the elements relevant to translation or protein folding include codon usage bias, codon adaptability, internal chi sites, ribosomal binding sites, premature polyA sites, Shine-Dalgarno sequences, codon context, codon-anticodon interactions, and translational pause sites;
wherein the modifications are made before the optimized DNA sequence is generated.

6.-7. (canceled)

8. The method of claim 1, wherein the method further comprises a step of synthesizing the optimized DNA sequence, and inserting the synthesized optimized DNA sequence in a nucleic acid vector for use in in vitro transcription to synthesize mRNA.

9. (canceled)

10. The method of claim 8, wherein the nucleic acid vector comprises an RNA polymerase promoter operably linked to the optimized DNA sequence, optionally wherein the RNA polymerase is SP6 RNA polymerase or a T7 RNA polymerase.

11.-19. (canceled)

20. The method of claim 1, wherein the method further comprises a step of capping and/or tailing the synthesized mRNA.

21. (canceled)

22. The method of claim 1, wherein the mRNA is synthesized in a reaction mixture comprising NTPs at a concentration ranging from 1-10 mM each NTP, the DNA template at a concentration ranging from 0.01-0.5 mg/ml, the SP6 RNA polymerase at a concentration ranging from 0.01-0.1 mg/ml, and at a temperature ranging from 37-56° C.

23.-25. (canceled)

26. The method of claim 22, wherein the NTPs comprise modified NTPs.

27-30. (canceled)

31. A method for preparing an optimized DNA sequence encoding a protein as a template for in vitro transcription, said method comprising:
a. providing a DNA sequence encoding a protein; and
b. adding one or more termination signals at the 3′ end of the DNA sequence to provide the optimized DNA sequence,
wherein the one or more termination signal(s) comprises the following nucleic acid sequence:
5′-X₁ATCTX₂TX₃-3′, wherein X₁, X₂ and X₃ are independently selected from A, C, T or G.

32-34. (canceled)

35. The method of claim 31, wherein the termination signal is selected from
5′-TTTT ATCTGTTTTTTT-3′,
5′-TTTTATCTGTTTTTTTTT-3′,
5′-CGTTTTATCTGTTTTTTT-3′,
5′-CGTTCCATCTGTTTTTTT-3′,
5′-CGTTTTATCTGTTTGTTT-3′,
5′-CGTTTTATCTGTTTGTTT-3′,
or
5 -CGTTTT ATCTGTTGTTTT-3′.

36. (canceled)

37. The method of claim 31, wherein the DNA sequence encoding the protein further comprises a first nucleic acid sequence encoding a 5′ UTR and/or a second nucleic acid sequence encoding a 3′ UTR.

38. The method of claim 37, wherein the DNA sequence encoding the protein further comprises a third nucleic acid sequence encoding a poly-A tail.

39. (canceled)

40. The method of claim 31 wherein the DNA sequence encoding the protein does not further comprise a DNA sequence encoding a ribozyme.

41. The method of claim 40, wherein the 5 nucleotides immediately 3′ of the termination signal in the DNA sequence encoding the protein do not comprise 3 or more T nucleotides.

42. (canceled)

43. The method of claim 31 wherein the optimized DNA sequence comprises the following sequence: (a) 5′-X1ATCTX2TX3-(Z_{N})—X₄ATCTX₅TX6-3′ or (b) 5′-X1ATCTX2TX3-(Z_{N})-X4ATCTX5TX6-(Z_{M})-X₇ATCTX₈TX9-3′, wherein X1, X₂, X₃, X₄, X₅, X6, X₇, X₈ and X₉ are independently selected from A, C, T or G, Z_{N} represents a spacer sequence of N nucleotides, and Z_{M} represents a spacer sequence of M nucleotides, each of which are independently selected from A, C, T or G, and wherein N and/or M are independently 10 or fewer.

44-50. (canceled)

51. A DNA sequence for use in in vitro transcription, comprising in 5′ to 3′ order:
a. A 5′UTR;
b. a protein coding sequence;
c. a 3′UTR;
d. optionally a nucleic acid sequence encoding a polyA tail; and
e. a termination signal;
wherein the termination signal comprises the following nucleic acid sequence:
5′-X₁ATCTX₂TX₃-3′, wherein X1, X₂ and X₃ are independently selected from A, C, T or G.

52-54. (canceled)

55. The DNA sequence of claim 51, wherein the termination signal is selected from 5′-TTTTATCTGTTTTTTT-3′, 5′-TTTTATCTGTTTTTTTTT-3′,′-CGTTTTATCTGTTTTTTT-3′, 5′-CGTTCCATCTGTTTTTTT-3′, 5′-CGTTTTATCTGTTTGTTT-3′, 5′-CGTTTTATCTGTTTGTTT-3′, or 5′-CGTTTT ATCTGTTGTTTT-3′, and wherein the termination signals are separated by 10 base pairs or fewer.

56-67. (canceled)

68. A kit for use in in vitro transcription comprising the DNA sequence of claim 51.

69. (canceled)

70. A method for the production of mRNA, said method comprising adding the nucleic acid vector comprising a DNA sequence of claim 51 to a reaction mixture comprising NTPs and an RNA polymerase, wherein the RNA polymerase transcribes the DNA sequence into mRNA transcripts.

71-97. (canceled)


SEQUENCE LISTING (38 sequences)

SEQ ID NO: 1 - DNA, 8 bp, Artificial Sequence
  Description of Artificial Sequence Synthetic oligonucleotide
  modified_base (1)..(1): a, c, t, or g
  modified_base (6)..(6): a, c, t, or g
  modified_base (8)..(8): a, c, t, or g
natctntn

SEQ ID NO: 2 - DNA, 8 bp, Artificial Sequence
  Description of Artificial Sequence Synthetic oligonucleotide
yatctgtt

SEQ ID NO: 3 - DNA, 16 bp, Artificial Sequence
  Description of Artificial Sequence Synthetic oligonucleotide
ttttatctgttttttt

SEQ ID NO: 4 - DNA, 18 bp, Artificial Sequence
  Description of Artificial Sequence Synthetic oligonucleotide
ttttatctgttttttttt

SEQ ID NO: 5 - DNA, 18 bp, Artificial Sequence
  Description of Artificial Sequence Synthetic oligonucleotide
cgttttatctgttttttt

SEQ ID NO: 6 - DNA, 18 bp, Artificial Sequence
  Description of Artificial Sequence Synthetic oligonucleotide
cgttccatctgttttttt

SEQ ID NO: 7 - DNA, 18 bp, Artificial Sequence
  Description of Artificial Sequence Synthetic oligonucleotide
cgttttatctgtttgttt

SEQ ID NO: 8 - DNA, 18 bp, Artificial Sequence
  Description of Artificial Sequence Synthetic oligonucleotide
cgttttatctgtttgttt

SEQ ID NO: 9 - DNA, 18 bp, Artificial Sequence
  Description of Artificial Sequence Synthetic oligonucleotide
cgttttatctgttgtttt

SEQ ID NO: 10 - DNA, 26 bp, Artificial Sequence
  Description of Artificial Sequence Synthetic oligonucleotide
  modified_base (1)..(1): a, c, t, or g
  modified_base (6)..(6): a, c, t, or g
  modified_base (8)..(19): a, c, t, or g
  misc_feature (9)..(18): This region may encompass 1-10 nucleotides
  modified_base (24)..(24): a, c, t, or g
  modified_base (26)..(26): a, c, t, or g
natctntnnnnnnnnnnnnatctntn

SEQ ID NO: 11 - DNA, 44 bp, Artificial Sequence
  Description of Artificial Sequence Synthetic oligonucleotide
  modified_base (1)..(1): a, c, t, or g
  modified_base (6)..(6): a, c, t, or g
  modified_base (8)..(19): a, c, t, or g
  misc_feature (9)..(18): This region may encompass 1-10 nucleotides
  modified_base (24)..(24): a, c, t, or g
  modified_base (26)..(37): a, c, t, or g
  misc_feature (27)..(36): This region may encompass 1-10 nucleotides
  modified_base (42)..(42): a, c, t, or g
  modified_base (44)..(44): a, c, t, or g
natctntnnnnnnnnnnnnatctntnnnnnnnnnnnnatctntn

SEQ ID NO: 12 - DNA, 36 bp, Artificial Sequence
  Description of Artificial Sequence Synthetic oligonucleotide
ttttatctgtttttttttttttatctgttttttttt

SEQ ID NO: 13 - DNA, 54 bp, Artificial Sequence
  Description of Artificial Sequence Synthetic oligonucleotide
ttttatctgtttttttttttttatctgtttttttttttttatctgttttttttt

SEQ ID NO: 14 - DNA, 19 bp, Artificial Sequence
  Description of Artificial Sequence Synthetic oligonucleotide
ttttatctgtttttttttt

SEQ ID NO: 15 - protein, 874 aa, Artificial Sequence
  Description of Artificial Sequence Synthetic bacteriophage SP6 sequence
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 16 - DNA, 2625 bp, Artificial Sequence
  Description of Artificial Sequence Synthetic bacteriophage SP6 sequence
atgcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagtgcgctggatggttgatacaggtatcgaagtacctgagcaaggggagttcgaccttaacgaaatcatggattctgaatacgtatttgcctaa

SEQ ID NO: 17 - DNA, 18 bp, Artificial Sequence
  Description of Artificial Sequence Synthetic bacteriophage SP6 sequence
atttaggtgacactatag

SEQ ID NO: 18 - DNA, 23 bp, Artificial Sequence
  Description of Artificial Sequence Synthetic oligonucleotide
atttaggggacactatagaagag

SEQ ID NO: 19 - DNA, 22 bp, Artificial Sequence
  Description of Artificial Sequence Synthetic oligonucleotide
atttaggggacactatagaagg

SEQ ID NO: 20 - DNA, 23 bp, Artificial Sequence
  Description of Artificial Sequence Synthetic oligonucleotide
atttaggggacactatagaaggg

SEQ ID NO: 21 - DNA, 20 bp, Artificial Sequence
  Description of Artificial Sequence Synthetic oligonucleotide
atttaggtgacactatagaa

SEQ ID NO: 22 - DNA, 22 bp, Artificial Sequence
  Description of Artificial Sequence Synthetic oligonucleotide
atttaggtgacactatagaaga

SEQ ID NO: 23 - DNA, 23 bp, Artificial Sequence
  Description of Artificial Sequence Synthetic oligonucleotide
atttaggtgacactatagaagag

SEQ ID NO: 24 - DNA, 22 bp, Artificial Sequence
  Description of Artificial Sequence Synthetic oligonucleotide
atttaggtgacactatagaagg

SEQ ID NO: 25 - DNA, 23 bp, Artificial Sequence
  Description of Artificial Sequence Synthetic oligonucleotide
atttaggtgacactatagaaggg

SEQ ID NO: 26 - DNA, 23 bp, Artificial Sequence
  Description of Artificial Sequence Synthetic oligonucleotide
  modified_base (22)..(22): a, c, t, or g
atttaggtgacactatagaagng

SEQ ID NO: 27 - DNA, 24 bp, Artificial Sequence
  Description of Artificial Sequence Synthetic oligonucleotide
catacgatttaggtgacactatag

SEQ ID NO: 28 - DNA, 18 bp, Artificial Sequence
  Description of Artificial Sequence Synthetic bacteriophage T7 sequence
taatacgactcactatag

SEQ ID NO: 29 - DNA, 15 bp, Artificial Sequence
  Description of Artificial Sequence Synthetic oligonucleotide
gctatctgttcatca

SEQ ID NO: 30 - DNA, 16 bp, Artificial Sequence
  Description of Artificial Sequence Synthetic oligonucleotide
  Description of Combined DNA/RNA Molecule Synthetic oligonucleotide
  modified_base (1)..(4): 2′OMe modified nucleotide
  modified_base (9)..(16): 2′OMe modified nucleotide
gaugcaacuuaauuuu

SEQ ID NO: 31 - DNA, 18 bp, Artificial Sequence
  Description of Artificial Sequence Synthetic oligonucleotide
  Description of Combined DNA/RNA Molecule Synthetic oligonucleotide
  modified_base (1)..(7): 2′OMe modified nucleotide
  modified_base (12)..(18): 2′OMe modified nucleotide
agcuugatgcaacuuaau

SEQ ID NO: 32 - DNA, 16 bp, Artificial Sequence
  Description of Artificial Sequence Synthetic oligonucleotide
  Description of Combined DNA/RNA Molecule Synthetic oligonucleotide
  modified_base (1)..(5): 2′OMe modified nucleotide
  modified_base (10)..(16): 2′OMe modified nucleotide
agcuugatgcaacuua

SEQ ID NO: 33 - DNA, 16 bp, Artificial Sequence
  Description of Artificial Sequence Synthetic oligonucleotide
  Description of Combined DNA/RNA Molecule Synthetic oligonucleotide
  modified_base (1)..(3): 2′OMe modified nucleotide
  modified_base (8)..(16): 2′OMe modified nucleotide
agcttgaugcaacuua

SEQ ID NO: 34 - DNA, 14 bp, Artificial Sequence
  Description of Artificial Sequence Synthetic oligonucleotide
  Description of Combined DNA/RNA Molecule Synthetic oligonucleotide
  modified_base (1)..(7): 2′OMe modified nucleotide
  modified_base (12)..(14): 2′OMe modified nucleotide
agcuugatgcaacu

SEQ ID NO: 35 - DNA, 11 bp, Artificial Sequence
  Description of Artificial Sequence Synthetic oligonucleotide
  Description of Combined DNA/RNA Molecule Synthetic oligonucleotide
  modified_base (1)..(5): 2′OMe modified nucleotide
  modified_base (10)..(11): 2′OMe modified nucleotide
cuugatgcaac

SEQ ID NO: 36 - DNA, 18 bp, Artificial Sequence
  Description of Artificial Sequence Synthetic oligonucleotide
tatctgttagtttttttc

SEQ ID NO: 37 - DNA, 10 bp, Escherichia coli
gtttgtcgtg

SEQ ID NO: 38 - protein, 6 aa, Artificial Sequence
  Description of Artificial Sequence Synthetic peptide
HHHHHH